SEQUENCE LISTING (57 sequences)

SEQ ID NO: 1 - protein, 426 aa, synthetic construct
GILGFVFTLGGGGSGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGSHSMRYFFTSVSRPGRGEPRFIAVGYVDDTQFVRFDSDAASQRMEPRAPWIEQEGPEYWDGETRKVKAHSQTHRVDLGTLRGYYNQSEAGSHTVQRMYGCDVGSDWRFLRGYHQYAYDGKDYIALKEDLRSWTAADMAAQTTKHKWEAAHVAEQLRAYLEGTCVEWLRRYLENGKETLQRTDAPKTHMTHHAVSDHEATLRCWALSFYPAEITLTWQRDGEDQTQDTELVETRPAGDGTFQKWAAVVVPSGQEQRYTCHVQHEGLPKPLTLRWEENLYFQGHHHHHH

SEQ ID NO: 2 - protein, 504 aa, synthetic construct
MSRSVALAVLALLSLSGLEAGILGFVFTLGGGGSGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGGGGSGSHSMRYFFTSVSRPGRGEPRFIAVGYVDDTQFVRFDSDAASQRMEPRAPWIEQEGPEYWDGETRKVKAHSQTHRVDLGTLRGYYNQSEAGSHTVQRMYGCDVGSDWRFLRGYHQYAYDGKDYIALKEDLRSWTAADMAAQTTKHKWEAAHVAEQLRAYLEGTCVEWLRRYLENGKETLQRTDAPKTHMTHHAVSDHEATLRCWALSFYPAEITLTWQRDGEDQTQDTELVETRPAGDGTFQKWAAVVVPSGQEQRYTCHVQHEGLPKPLTLRWEPSSQPTIPIVGIIAGLVLFGAVITGAVVAAVMWRRKSSDRKGGSYSQAASSDSAQGSDVSLTACKV

SEQ ID NO: 3 - protein, 431 aa, synthetic construct
GILGFVFTLGGGGSGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGGGGSGSHSMRYFFTSVSRPGRGEPRFIAVGYVDDTQFVRFDSDAASQRMEPRAPWIEQEGPEYWDGETRKVKAHSQTHRVDLGTLRGYYNQSEAGSHTVQRMYGCDVGSDWRFLRGYHQYAYDGKDYIALKEDLRSWTAADMAAQTTKHKWEAAHVAEQLRAYLEGTCVEWLRRYLENGKETLQRTDAPKTHMTHHAVSDHEATLRCWALSFYPAEITLTWQRDGEDQTQDTELVETRPAGDGTFQKWAAVVVPSGQEQRYTCHVQHEGLPKPLTLRWEENLYFQGHHHHHH

SEQ ID NO: 4 - protein, 431 aa, synthetic construct; identical to SEQ ID NO: 3

SEQ ID NO: 5 - protein, 428 aa, synthetic construct
SYRPGTVALGGGGSGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSCSHSMRYFDTAVSRPGRGEPRFISVGYVDDTQFVRFDSDAASPRGEPRAPWVEQEGPEYWDRETQKYKRQAQADRVSLRNLRGYYNQSEDGSHTLQRMSGCDLGPDGRLLRGYDQSAYDGKDYIALNEDLRSWTAADTAAQITQRKLEAARAAEQLRAYLEGTCVEWLRRYLENGKETLQRAEPPKTHVTHHPLSDHEATLRCWALGFYPAEITLTWQRDGEDQTQDTELVETRPAGDGTFQKWAAVVVPSGQEQRYTCHMQHEGLQEPLTLSWEPSENLYFQGHHHHHH

SEQ ID NO: 6 - protein, 428 aa, synthetic construct
SYRPGTVALGGGGSGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGSHSMRYFDTAVSRPGRGEPRFISVGYVDDTQFVRFDSDAASPRGEPRAPWVEQEGPEYWDRETQKYKRQAQADRVSLRNLRGYYNQSEDGSHTLQRMSGCDLGPDGRLLRGYDQSAYDGKDYIALNEDLRSWTAADTAAQITQRKLEAARAAEQLRAYLEGTCVEWLRRYLENGKETLQRAEPPKTHVTHHPLSDHEATLRCWALGFYPAEITLTWQRDGEDQTQDTELVETRPAGDGTFQKWAAVVVPSGQEQRYTCHMQHEGLQEPLTLSWEPSENLYFQGHHHHHH

SEQ ID NO: 7 - protein, 480 aa, synthetic construct
MSRSVALAVLALLSLSGLEAIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGGGGSGSHSMRYFFTSVSRPGRGEPRFIAVGYVDDTQFVRFDSDAASQRMEPRAPWIEQEGPEYWDGETRKVKAHSQTHRVDLGTLRGYYNQSEAGSHTVQRMYGCDVGSDWRFLRGYHQYAYDGKDYIALKEDLRSWTAADMAAQTTKHKWEAAHVAEQLRAYLEGTCVEWLRRYLENGKETLQRTDAPKTHMTHHAVSDHEATLRCWALSFYPAEITLTWQRDGEDQTQDTELVETRPAGDGTFQKWAAVVVPSGQEQRYTCHVQHEGLPKPLTLRWEPSSQPTIPIVGIIAGLVLFGAVITGAVVAAVMWRRKSSDRKGGSYSQAASSDSAQGSDVSLTACKV

SEQ ID NO: 8 - protein, 118 aa, synthetic construct
SYRPGTVALGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDM

SEQ ID NO: 9 - protein, 118 aa, synthetic construct
SYRPGTVALGCGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDM

SEQ ID NO: 10 - protein, 120 aa, synthetic construct
SYRPGTVALGGGGSGGGGSGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDM

SEQ ID NO: 11 - protein, 120 aa, synthetic construct
SYRPGTVALGCGGSGGGGSGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDM

SEQ ID NO: 12 - protein, 123 aa, synthetic construct
SYRPGTVALGGGGSGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDM

SEQ ID NO: 13 - protein, 123 aa, synthetic construct
SYRPGTVALGCGGSGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDM

SEQ ID NO: 14 - protein, 426 aa, synthetic construct
GILGFVFTLGGGGSGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGSHSMRYFFTSVSRPGRGEPRFIAVGYVDDTQFVRFDSDAASQRMEPRAPWIEQEGPEYWDGETRKVKAHSQTHRVDLGTLRGAYNQSEAGSHTVQRMYGCDVGSDWRFLRGYHQYAYDGKDYIALKEDLRSWTAADMAAQTTKHKWEAAHVAEQLRAYLEGTCVEWLRRYLENGKETLQRTDAPKTHMTHHAVSDHEATLRCWALSFYPAEITLTWQRDGEDQTQDTELVETRPAGDGTFQKWAAVVVPSGQEQRYTCHVQHEGLPKPLTLRWEENLYFQGHHHHHH

SEQ ID NO: 15 - protein, 428 aa, synthetic construct
RYRPGTVALGGGGSGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSCSHSMRYFDTAVSRPGRGEPRFISVGYVDDTQFVRFDSDAASPRGEPRAPWVEQEGPEYWDRETQKYKRQAQADRVSLRNLRGAYNQSEDGSHTLQRMSGCDLGPDGRLLRGYDQSAYDGKDYIALNEDLRSWTAADTAAQITQRKLEAARAAEQLRAYLEGTCVEWLRRYLENGKETLQRAEPPKTHVTHHPLSDHEATLRCWALGFYPAEITLTWQRDGEDQTQDTELVETRPAGDGTFQKWAAVVVPSGQEQRYTCHMQHEGLQEPLTLSWEPSENLYFQGHHHHHH

SEQ ID NO: 16 - protein, 428 aa, synthetic construct; identical to SEQ ID NO: 15

SEQ ID NO: 17 - protein, 428 aa, synthetic construct; identical to SEQ ID NO: 15

SEQ ID NO: 18 - protein, 425 aa, synthetic construct
RYRPGTVALGGGGSGGGGSGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSCSHSMRYFDTAVSRPGRGEPRFISVGYVDDTQFVRFDSDAASPRGEPRAPWVEQEGPEYWDRETQKYKRQAQADRVSLRNLRGAYNQSEDGSHTLQRMSGCDLGPDGRLLRGYDQSAYDGKDYIALNEDLRSWTAADTAAQITQRKLEAARAAEQLRAYLEGTCVEWLRRYLENGKETLQRAEPPKTHVTHHPLSDHEATLRCWALGFYPAEITLTWQRDGEDQTQDTELVETRPAGDGTFQKWAAVVVPSGQEQRYTCHMQHEGLQEPLTLSWEPSENLYFQGHHHHHH

SEQ ID NO: 19 - protein, 425 aa, synthetic construct; identical to SEQ ID NO: 18

SEQ ID NO: 20 - protein, 425 aa, synthetic construct; identical to SEQ ID NO: 18

SEQ ID NO: 21 - protein, 425 aa, synthetic construct; identical to SEQ ID NO: 18

SEQ ID NO: 22 - protein, 425 aa, synthetic construct; identical to SEQ ID NO: 18

SEQ ID NO: 23 - protein, 425 aa, synthetic construct; identical to SEQ ID NO: 18

SEQ ID NO: 24 - protein, 428 aa, synthetic construct
SYRPGTVALGGGGSGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSCSHSMRYFDTAVSRPGRGEPRFISVGYVDDTQFVRFDSDAASPRGEPRAPWVEQEGPEYWDRETQKYKRQAQADRVSLRNLRGAYNQSEDGSHTLQRMSGCDLGPDGRLLRGYDQSAYDGKDYIALNEDLRSWTAADTAAQITQRKLEAARAAEQLRAYLEGTCVEWLRRYLENGKETLQRAEPPKTHVTHHPLSDHEATLRCWALGFYPAEITLTWQRDGEDQTQDTELVETRPAGDGTFQKWAAVVVPSGQEQRYTCHMQHEGLQEPLTLSWEPSENLYFQGHHHHHH

SEQ ID NO: 25 - protein, 428 aa, synthetic construct
SYRPGTVALGGGGSGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSCSHSMRYFDTAVSRPGRGEPRFISVGYVDDTQFVRFDSDAASPRGEPRAPWVEQEGPEYWDRETQKYKRQAQADRVSLRNLRGAYNQSEDGSHTLQRMSGCDLGPDGRLLRGYDQSAYDGKDYIALNEDLRSWTAADTAAQITQRKLEAARAAEQLRAYLEGTCVEWLRRYLENGKETLQRAEPPKTHVTHHPLSDHEATLRCWALGFYPAEITLTWQRDGEDQTAKTELVETRPAGDGTFQKWAAVVVPSGQEQRYTCHMQHEGLQEPLTLSWEPSENLYFQGHHHHHH

SEQ ID NO: 26 - protein, 428 aa, synthetic construct
SYRPGTVALGCGGSGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSCSHSMRYFDTAVSRPGRGEPRFISVGYVDDTQFVRFDSDAASPRGEPRAPWVEQEGPEYWDRETQKYKRQAQADRVSLRNLRGCYNQSEDGSHTLQRMSGCDLGPDGRLLRGYDQSAYDGKDYIALNEDLRSWTAADTAAQITQRKLEAARAAEQLRAYLEGTCVEWLRRYLENGKETLQRAEPPKTHVTHHPLSDHEATLRCWALGFYPAEITLTWQRDGEDQTAKTELVETRPAGDGTFQKWAAVVVPSGQEQRYTCHMQHEGLQEPLTLSWEPSENLYFQGHHHHHH

SEQ ID NO: 27 - protein, 425 aa, synthetic construct
SYRPGTVALGGGGSGGGGSGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSCSHSMRYFDTAVSRPGRGEPRFISVGYVDDTQFVRFDSDAASPRGEPRAPWVEQEGPEYWDRETQKYKRQAQADRVSLRNLRGAYNQSEDGSHTLQRMSGCDLGPDGRLLRGYDQSAYDGKDYIALNEDLRSWTAADTAAQITQRKLEAARAAEQLRAYLEGTCVEWLRRYLENGKETLQRAEPPKTHVTHHPLSDHEATLRCWALGFYPAEITLTWQRDGEDQTQDTELVETRPAGDGTFQKWAAVVVPSGQEQRYTCHMQHEGLQEPLTLSWEPSENLYFQGHHHHHH

SEQ ID NO: 28 - protein, 425 aa, synthetic construct
SYRPGTVALGGGGSGGGGSGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSCSHSMRYFDTAVSRPGRGEPRFISVGYVDDTQFVRFDSDAASPRGEPRAPWVEQEGPEYWDRETQKYKRQAQADRVSLRNLRGAYNQSEDGSHTLQRMSGCDLGPDGRLLRGYDQSAYDGKDYIALNEDLRSWTAADTAAQITQRKLEAARAAEQLRAYLEGTCVEWLRRYLENGKETLQRAEPPKTHVTHHPLSDHEATLRCWALGFYPAEITLTWQRDGEDQTAKTELVETRPAGDGTFQKWAAVVVPSGQEQRYTCHMQHEGLQEPLTLSWEPSENLYFQGHHHHHH

SEQ ID NO: 29 - protein, 425 aa, synthetic construct
SYRPGTVALGCGGSGGGGSGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSCSHSMRYFDTAVSRPGRGEPRFISVGYVDDTQFVRFDSDAASPRGEPRAPWVEQEGPEYWDRETQKYKRQAQADRVSLRNLRGCYNQSEDGSHTLQRMSGCDLGPDGRLLRGYDQSAYDGKDYIALNEDLRSWTAADTAAQITQRKLEAARAAEQLRAYLEGTCVEWLRRYLENGKETLQRAEPPKTHVTHHPLSDHEATLRCWALGFYPAEITLTWQRDGEDQTAKTELVETRPAGDGTFQKWAAVVVPSGQEQRYTCHMQHEGLQEPLTLSWEPSENLYFQGHHHHHH

SEQ ID NO: 30 - protein, 423 aa, synthetic construct
SYRPGTVALGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSCSHSMRYFDTAVSRPGRGEPRFISVGYVDDTQFVRFDSDAASPRGEPRAPWVEQEGPEYWDRETQKYKRQAQADRVSLRNLRGAYNQSEDGSHTLQRMSGCDLGPDGRLLRGYDQSAYDGKDYIALNEDLRSWTAADTAAQITQRKLEAARAAEQLRAYLEGTCVEWLRRYLENGKETLQRAEPPKTHVTHHPLSDHEATLRCWALGFYPAEITLTWQRDGEDQTQDTELVETRPAGDGTFQKWAAVVVPSGQEQRYTCHMQHEGLQEPLTLSWEPSENLYFQGHHHHHH

SEQ ID NO: 31 - protein, 423 aa, synthetic construct
SYRPGTVALGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSCSHSMRYFDTAVSRPGRGEPRFISVGYVDDTQFVRFDSDAASPRGEPRAPWVEQEGPEYWDRETQKYKRQAQADRVSLRNLRGAYNQSEDGSHTLQRMSGCDLGPDGRLLRGYDQSAYDGKDYIALNEDLRSWTAADTAAQITQRKLEAARAAEQLRAYLEGTCVEWLRRYLENGKETLQRAEPPKTHVTHHPLSDHEATLRCWALGFYPAEITLTWQRDGEDQTAKTELVETRPAGDGTFQKWAAVVVPSGQEQRYTCHMQHEGLQEPLTLSWEPSENLYFQGHHHHHH

SEQ ID NO: 32 - protein, 423 aa, synthetic construct
SYRPGTVALGCGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSCSHSMRYFDTAVSRPGRGEPRFISVGYVDDTQFVRFDSDAASPRGEPRAPWVEQEGPEYWDRETQKYKRQAQADRVSLRNLRGCYNQSEDGSHTLQRMSGCDLGPDGRLLRGYDQSAYDGKDYIALNEDLRSWTAADTAAQITQRKLEAARAAEQLRAYLEGTCVEWLRRYLENGKETLQRAEPPKTHVTHHPLSDHEATLRCWALGFYPAEITLTWQRDGEDQTAKTELVETRPAGDGTFQKWAAVVVPSGQEQRYTCHMQHEGLQEPLTLSWEPSENLYFQGHHHHHH

SEQ ID NO: 33 - protein, 428 aa, synthetic construct
SYRPGTVALGGGGSGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGSHSMRYFDTAVSRPGRGEPRFISVGYVDDTQFVRFDSDAASPRGEPRAPWVEQEGPEYWDRETQKYKRQAQADRVSLRNLRGAYNQSEDGSHTLQRMSGCDLGPDGRLLRGYDQSAYDGKDYIALNEDLRSWTAADTAAQITQRKLEAARAAEQLRAYLEGTCVEWLRRYLENGKETLQRAEPPKTHVTHHPLSDHEATLRCWALGFYPAEITLTWQRDGEDQTQDTELVETRPAGDGTFQKWAAVVVPSGQEQRYTCHMQHEGLQEPLTLSWEPSENLYFQGHHHHHH

SEQ ID NO: 34 - protein, 428 aa, synthetic construct
SYRPGTVALGGGGSGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGSHSMRYFDTAVSRPGRGEPRFISVGYVDDTQFVRFDSDAASPRGEPRAPWVEQEGPEYWDRETQKYKRQAQADRVSLRNLRGAYNQSEDGSHTLQRMSGCDLGPDGRLLRGYDQSAYDGKDYIALNEDLRSWTAADTAAQITQRKLEAARAAEQLRAYLEGTCVEWLRRYLENGKETLQRAEPPKTHVTHHPLSDHEATLRCWALGFYPAEITLTWQRDGEDQTAKTELVETRPAGDGTFQKWAAVVVPSGQEQRYTCHMQHEGLQEPLTLSWEPSENLYFQGHHHHHH

SEQ ID NO: 35 - protein, 428 aa, synthetic construct
SYRPGTVALGCGGSGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGSHSMRYFDTAVSRPGRGEPRFISVGYVDDTQFVRFDSDAASPRGEPRAPWVEQEGPEYWDRETQKYKRQAQADRVSLRNLRGCYNQSEDGSHTLQRMSGCDLGPDGRLLRGYDQSAYDGKDYIALNEDLRSWTAADTAAQITQRKLEAARAAEQLRAYLEGTCVEWLRRYLENGKETLQRAEPPKTHVTHHPLSDHEATLRCWALGFYPAEITLTWQRDGEDQTAKTELVETRPAGDGTFQKWAAVVVPSGQEQRYTCHMQHEGLQEPLTLSWEPSENLYFQGHHHHHH

SEQ ID NO: 36 - protein, 425 aa, synthetic construct
SYRPGTVALGGGGSGGGGSGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGSHSMRYFDTAVSRPGRGEPRFISVGYVDDTQFVRFDSDAASPRGEPRAPWVEQEGPEYWDRETQKYKRQAQADRVSLRNLRGAYNQSEDGSHTLQRMSGCDLGPDGRLLRGYDQSAYDGKDYIALNEDLRSWTAADTAAQITQRKLEAARAAEQLRAYLEGTCVEWLRRYLENGKETLQRAEPPKTHVTHHPLSDHEATLRCWALGFYPAEITLTWQRDGEDQTQDTELVETRPAGDGTFQKWAAVVVPSGQEQRYTCHMQHEGLQEPLTLSWEPSENLYFQGHHHHHH

SEQ ID NO: 37 - protein, 425 aa, synthetic construct
SYRPGTVALGGGGSGGGGSGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGSHSMRYFDTAVSRPGRGEPRFISVGYVDDTQFVRFDSDAASPRGEPRAPWVEQEGPEYWDRETQKYKRQAQADRVSLRNLRGAYNQSEDGSHTLQRMSGCDLGPDGRLLRGYDQSAYDGKDYIALNEDLRSWTAADTAAQITQRKLEAARAAEQLRAYLEGTCVEWLRRYLENGKETLQRAEPPKTHVTHHPLSDHEATLRCWALGFYPAEITLTWQRDGEDQTAKTELVETRPAGDGTFQKWAAVVVPSGQEQRYTCHMQHEGLQEPLTLSWEPSENLYFQGHHHHHH

SEQ ID NO: 38 - protein, 425 aa, synthetic construct; identical to SEQ ID NO: 37

SEQ ID NO: 39 - protein, 423 aa, synthetic construct
SYRPGTVALGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGSHSMRYFDTAVSRPGRGEPRFISVGYVDDTQFVRFDSDAASPRGEPRAPWVEQEGPEYWDRETQKYKRQAQADRVSLRNLRGAYNQSEDGSHTLQRMSGCDLGPDGRLLRGYDQSAYDGKDYIALNEDLRSWTAADTAAQITQRKLEAARAAEQLRAYLEGTCVEWLRRYLENGKETLQRAEPPKTHVTHHPLSDHEATLRCWALGFYPAEITLTWQRDGEDQTQDTELVETRPAGDGTFQKWAAVVVPSGQEQRYTCHMQHEGLQEPLTLSWEPSENLYFQGHHHHHH

SEQ ID NO: 40 - protein, 423 aa, synthetic construct; identical to SEQ ID NO: 39

SEQ ID NO: 41 - protein, 423 aa, synthetic construct
SYRPGTVALGCGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGSHSMRYFDTAVSRPGRGEPRFISVGYVDDTQFVRFDSDAASPRGEPRAPWVEQEGPEYWDRETQKYKRQAQADRVSLRNLRGCYNQSEDGSHTLQRMSGCDLGPDGRLLRGYDQSAYDGKDYIALNEDLRSWTAADTAAQITQRKLEAARAAEQLRAYLEGTCVEWLRRYLENGKETLQRAEPPKTHVTHHPLSDHEATLRCWALGFYPAEITLTWQRDGEDQTAKTELVETRPAGDGTFQKWAAVVVPSGQEQRYTCHMQHEGLQEPLTLSWEPSENLYFQGHHHHHH

SEQ ID NO: 42 - protein, 290 aa, synthetic construct
GSHSMRYFDTAVSRPGRGEPRFISVGYVDDTQFVRFDSDAASPRGEPRAPWVEQEGPEYWDRETQKYKRQAQADRVSLRNLRGAYNQSEDGSHTLQRMSGCDLGPDGRLLRGYDQSAYDGKDYIALNEDLRSWTAADTAAQITQRKLEAARAAEQLRAYLEGTCVEWLRRYLENGKETLQRAEPPKTHVTHHPLSDHEATLRCWALGFYPAEITLTWQRDGEDQTQDTELVETRPAGDGTFQKWAAVVVPSGQEQRYTCHMQHEGLQEPLTLSWEPSENLYFQGHHHHHH

SEQ ID NO: 43 - protein, 290 aa, synthetic construct
GSHSMRYFDTAVSRPGRGEPRFISVGYVDDTQFVRFDSDAASPRGEPRAPWVEQEGPEYWDRETQKYKRQAQADRVSLRNLRGAYNQSEDGSHTLQRMSGCDLGPDGRLLRGYDQSAYDGKDYIALNEDLRSWTAADTAAQITQRKLEAARAAEQLRAYLEGTCVEWLRRYLENGKETLQRAEPPKTHVTHHPLSDHEATLRCWALGFYPAEITLTWQRDGEDQTAKTELVETRPAGDGTFQKWAAVVVPSGQEQRYTCHMQHEGLQEPLTLSWEPSENLYFQGHHHHHH

SEQ ID NO: 44 - protein, 290 aa, synthetic construct
GSHSMRYFDTAVSRPGRGEPRFISVGYVDDTQFVRFDSDAASPRGEPRAPWVEQEGPEYWDRETQKYKRQAQADRVSLRNLRGCYNQSEDGSHTLQRMSGCDLGPDGRLLRGYDQSAYDGKDYIALNEDLRSWTAADTAAQITQRKLEAARAAEQLRAYLEGTCVEWLRRYLENGKETLQRAEPPKTHVTHHPLSDHEATLRCWALGFYPAEITLTWQRDGEDQTAKTELVETRPAGDGTFQKWAAVVVPSGQEQRYTCHMQHEGLQEPLTLSWEPSENLYFQGHHHHHH

SEQ ID NO: 45 - protein, 9 aa, Influenza A virus
GILGFVFTL

SEQ ID NO: 46 - protein, 9 aa, Homo sapiens
RYRPGTVAL

SEQ ID NO: 47 - protein, 9 aa, synthetic construct
SYRPGTVAL

SEQ ID NO: 48 - protein, 15 aa, synthetic construct
GGGGSGGGGSGGGGS

SEQ ID NO: 49 - protein, 20 aa, synthetic construct
GGGGSGGGGSGGGGSGGGGS

SEQ ID NO: 50 - protein, 10 aa, synthetic construct
GGGGSGGGGS

SEQ ID NO: 51 - protein, 10 aa, synthetic construct
GCGGSGGGGS

SEQ ID NO: 52 - protein, 12 aa, synthetic construct
GGGGSGGGGSGS

SEQ ID NO: 53 - protein, 12 aa, synthetic construct
GCGGSGGGGSGS

SEQ ID NO: 54 - protein, 15 aa, synthetic construct
GCGGSGGGGSGGGGS

SEQ ID NO: 55 - protein, 15 aa, synthetic construct
GLNDIFEAQKIEWHE

SEQ ID NO: 56 - protein, 15 aa, synthetic construct
GLNDIFEAQKIEWHE

SEQ ID NO: 57 - protein, 15 aa, synthetic construct
GLNDIFEAQKIEWHE

(19) United States
(12) Patent Application Publication
(10) Pub. No.: US 2024/0124551 A1
(43) Pub. Date: Apr. 18, 2024

(54) METHODS AND COMPOSITIONS FOR CELLULAR THERAPY

(71) Applicant: Replay Holdings, Inc., San Diego, CA (US)

(72) Inventors: Herman WALDMANN, Oxford (GB); Ashley BUCKLE, Mulgrave (AU); Adrian WOOLFSON, San Francisco, CA (US)

(21) Appl. No.: 18/303,075

(22) Filed: Apr. 19, 2023

Related U.S. Application Data
(63) Continuation of application No. PCT/US2021/055682, filed on Oct. 19, 2021.

(30) Foreign Application Priority Data
Oct. 20, 2020 (GB) 2016659.1
Feb. 5, 2021 (GB) 2101665.4

(51) Int. Cl. C07K 14/74; C07K 14/705; C12N 15/74; C12P 21/02

(52) U.S. Cl. CPC C07K 14/70539; C07K 14/70503; C07K 14/70521; C07K 14/70532; C12N 15/74; C12P 21/02; C12N 2800/101

(57) ABSTRACT

Provided herein is a synthetic complex comprising one or more human leukocyte antigens (synHLA), wherein said complex is inhibited from eliciting an immune response. Also provided are a nucleic acid molecule encoding said complex, an immune incompetent stem cell comprising said complex or said nucleic acid molecule, and a method of treating a disease or disorder comprising administering said complex, said nucleic acid molecule, or said immune incompetent stem cell to a subject in need thereof.

### CROSS-REFERENCE

This application is a continuation of International Application No. PCT/US2021/055682, filed Oct. 19, 2021, which claims the benefit of UK Patent Application No. 2016659.1, filed Oct. 20, 2020, and UK Patent Application No. 2101665.4, filed Feb. 5, 2021, both of which applications are incorporated herein by reference in their entirety.

### SEQUENCE LISTING

The instant application contains a Sequence Listing which has been submitted electronically in XML format and is hereby incorporated by reference in its entirety. Said XML copy, created on Apr. 18, 2023, is named 62600-702_301_SL.xml and is 66,717 bytes in size.

### BACKGROUND OF THE INVENTION

Cellular therapies hold great promise for combating previously intractable diseases. While the use of autologous cells is generally optimal, this approach can be cost-prohibitive and burdensome. Although allogeneic pluripotent stem cells (PSCs) provide greater scalability and cost savings, their utility is limited by the need to match human leukocyte antigen (HLA) class 1 alleles, the most genetically polymorphic region in the human genome. Mismatches in HLA class 1 haplotypes lead to the “self versus non-self” immune response that can result in the body's rejection of transplanted therapeutic cells. The general utility of recent efforts to engineer HLA complexes that are blocked from triggering a T-cell-activated immune response has been limited by the failure of these complexes to successfully engage the killer-cell immunoglobulin-like receptor (KIR), resulting in a “missing self” immune response. Consequently, there remains an unmet need for the development of engineered pluripotent stem cells that circumvent both T-cell- and MR-mediated immune responses.

### SUMMARY OF THE INVENTION

Provided herein, in an aspect, is a complex comprising one or more human leukocyte antigens (HLAs), wherein the one or more HLAs are inhibited from eliciting a T-cell response when the complex is interrogated by one or more T-cells.

In some embodiments, the complex comprises, in N-terminus to C-terminus order, a segment comprising a peptide and a segment comprising a human HLA class 1 heavy chain sequence. In some embodiments, the peptide does not elicit a T-cell response when the peptide is interrogated by one or more T-cells. In some embodiments, the peptide is incapable of activating the one or more T-cells. In some embodiments, the peptide is capable of binding to a receptor of the one or more T-cells, and the binding is insufficient to activate the one or more T-cells. In some embodiments, the peptide binds to one or more HLA binding groove domain residues of the human HLA class 1 heavy chain sequence. In some embodiments, the peptide modulates a conformation of the human HLA class 1 heavy chain sequence. In some embodiments, the conformation prevents the one or more T-cells from binding to the human HLA class 1 heavy chain sequence. In some embodiments, the peptide comprises greater than or equal to 8, 9, 10, 11, 12, 13, or 14 amino acids. In some embodiments, the human HLA class 1 heavy chain sequence comprises one or more class 1 HLAs.

The complex of any one of claims 2 to 10, wherein the human HLA class 1 heavy chain sequence comprises HLA-A, HLA-B, HLA-C, or any combination thereof. In some embodiments, the human HLA class 1 heavy chain sequence comprise multiple versions of HLA-A, HLA-B, HLA-C, or any combination thereof. In some embodiments, the human HLA class 1 heavy chain sequence comprises the HLA-A, wherein the HLA-A is displaced between the HLA-B and the HLA-C. In some embodiments, the complex further comprises one or more linkers between the peptide and the human HLA class 1 heavy chain sequence. In some embodiments, the one or more linkers are configured to resist proteolytic cleavage. In some embodiments, the one or more linkers comprise a conformation configured to not block one or more killer-cell immunoglobulin-like receptor (KIR) binding sites on the human HLA class 1 heavy chain sequence. In some embodiments, the peptide is coupled to the complex by a disulfide bond. In some embodiments, the complex further comprises one or more immune checkpoint agonists. In some embodiments, the one or more immune checkpoint agonists comprise CD47, PD-L1, A2AR, B7-H3, B7-H4, BTLA, CTLA-4, IDO, KIR, LAG3, NOX2, PD-1, TIM-3, VISTA, SIGLEC7, or combination thereof. In some embodiments, the human HLA class 1 heavy chain sequence comprises HLA-E or a fragment thereof, HLA-F or a fragment thereof, HLA-G or a fragment thereof, or any combination thereof. In some embodiments, at least one of the HLA-E or the fragment thereof, HLA-F or the fragment thereof, HLA-G or the fragment thereof, or any combination thereof is inhibited from eliciting a T-cell response when the complex is interrogated by one or more T-cells. In some embodiments, the complex further comprises a regulatory peptide. In some embodiments, the regulatory peptide is an apoptosis-inducing peptide. In some embodiments, the complex further comprises an epitope configured to allow for detection of the complex. In some embodiments, the epitope comprises 3,5-dinitrosalicylic acid. In some embodiments, the complex comprises a human β2-microglobulin sequence. In some embodiments, a linker of the one or more linkers is displaced between the peptide and the human β2-microglobulin sequence, between the human β2-microglobulin sequence and the human HLA class 1 heavy chain sequence, or both. In some embodiments, a linker of the one or more linkers comprises a sequence at least about 70%, 80%, 90%, or 99% identical to any one of SEQ ID NOs: 48-54. In some embodiments, the complex comprises, in N-terminus to C-terminus order,
a. the peptide;
b. a first linker of the one or more linkers;
c. the human β2-microglobulin sequence;
d. a second linker of the one or more linkers; and
e. the human HLA class 1 heavy chain sequence.

Provided herein, in another aspect, is a complex comprising one or more human leukocyte antigens (HLAs), wherein the one or more HLAs are inhibited from eliciting a T-cell response when the complex is interrogated by one or more T-cells, and wherein the complex comprises,
a. a first linker; and
b. a segment comprising a human HLA class 1 heavy chain sequence;
   wherein the first linker comprises a conformation configured to not block one or more killer-cell immunoglobulin-like receptor (KIR) binding sites on the human HLA class 1 heavy chain sequence.

In some embodiments, the complex comprises a peptide, wherein the peptide is configured or selected for being incapable of activating the one or more T-cells. In some embodiments, the configuration is further configured to resist proteolytic cleavage. In some embodiments, the complex further comprises a human β2-microglobulin and an additional linker between the human β2-microglobulin sequence and the human HLA class 1 heavy chain sequence. In some embodiments, the additional linker comprises a conformation configured to resist proteolytic cleavage. In some embodiments, the additional linker is further configured to not block one or more killer-cell immunoglobulin-like receptor (KIR) binding sites on the human HLA class 1 heavy chain sequence. In some embodiments, the first linker comprises a sequence at least about 70%, 80%, 90%, or 99% identical to any one of SEQ ID NOs: 48-54 or the additional linker comprises a sequence at least about 70%, 80%, 90%, or 99% identical to any one of SEQ ID NOs: 48-54. In some embodiments, the one or more human leukocyte antigens (HLAs) comprise one or more mutations, wherein the one or more mutations inhibit the one or more HLAs from eliciting a T-cell response when the complex is interrogated by one or more CD8 cells. In some embodiments, the one or more mutations comprises a mutation of one or more of amino acid residues 115, 122, 128, 194, 197, 198, 212, 214, 222, 223, 224, 225, 226, 227, 228, 229, 230, 231, 232, 233, 243, 245, 248, 262, or any combination thereof. In some embodiments, the complex further comprises one or more proteins or fragments thereof that inhibit an immune response by the complement system. In some embodiments, the one or more proteins or fragments thereof are selected from CD48, CD59, or a combination thereof. In some embodiments, the peptide comprises a second amino acid residue selected from L, M, S, I, F, T, V, and Y. In some embodiments, the second amino acid residue is selected from T, V, and Y. In some embodiments, the peptide comprises a last amino acid residue selected from V, I, F, W, Y, L, R, and K. In some embodiments, the last amino acid residue is selected from Y, L, R, and K. In some embodiments, the peptide comprises a second amino acid residue selected from E, P, L, Q, A, R, H, S, T, V, M, D, and K. In some embodiments, the second amino acid residue is selected from E, P, L, Q, A, R, and H. In some embodiments, the peptide comprises a last amino acid residue selected from V, L, F, A, I, Y, M, W, P, and R. In some embodiments, the last amino acid residue is selected from V, L, and F. In some embodiments, the peptide comprises a second amino acid residue selected from A, Y, S, T, V, I, L, F, Q, R, N, and W. In some embodiments, the second amino acid residue is selected from A and Y. In some embodiments, the peptide comprises a last amino acid residue selected from L, V, M, F, Y, and I. In some embodiments, the last amino acid residue is L.

Provided herein, in another aspect, is a nucleic acid molecule encoding a complex as described herein.

In some embodiments, the nucleic acid molecule comprises a deletion in the endogenous HLA locus. In some embodiments, the deletion comprises a deletion in the endogenous HLA-A, HLA-B, or HLA-C locus, or any combination thereof. In some embodiments, the deletion is complete deletion of the endogenous HLA locus. In some embodiments, the nucleic acid molecule further comprises a sequence encoding a human HLA class 1 heavy chain sequence. In some embodiments, the sequence encoding a human HLA class 1 heavy chain sequence comprises an HLA-A sequence, an HLA-B sequence, an HLA-C sequence, or any combination thereof. In some embodiments, the sequence encoding a human HLA class 1 heavy chain sequence comprises multiple alleles of an HLA-A sequence, an HLA-B sequence, an HLA-C sequence, or any combination thereof. In some embodiments, the sequence encoding a human HLA class 1 heavy chain sequence comprises an HLA-A sequence, wherein the HLA-A sequence is displaced between the HLA-B sequence and the HLA-C sequence. In some embodiments, the sequence encoding a human HLA class 1 heavy chain sequence comprises fewer than 1700 base pairs (bp). In some embodiments, the sequence encoding a human HLA class 1 heavy chain sequence comprises fewer than 500 bp. In some embodiments, the sequence encoding a human HLA class 1 heavy chain sequence comprises fewer than 250 bp. In some embodiments, the sequence encoding a human HLA class 1 heavy chain sequence comprises fewer than 150 bp. In some embodiments, the HLA-A sequence, HLA-B sequence, HLA-C sequences, or combination thereof comprises one or more flanking sequences. In some embodiments, the one or more flanking sequences comprise an endogenous HLA sequence. In some embodiments, the one or more flanking sequences are specific to one or more promoters. In some embodiments, the promoters comprise an HLA-A promoter, HLA-B promoter, HLA-C promoter, or combination thereof. In some embodiments, the sequence encoding a human HLA class 1 heavy chain sequence does not comprise at least a portion of the HLA-A sequence, HLA-B sequence, HLA-C sequence, or combination thereof. In some embodiments, the nucleic acid molecule further comprises a sequence encoding a human β2-microglobulin peptide. In some embodiments, the nucleic acid molecule further comprises a sequence encoding a peptide. In some embodiments, the peptide does not elicit a T-cell response when the peptide is interrogated by one or more T-cells. In some embodiments, the peptide is incapable of activating the one or more T-cells. In some embodiments, the peptide is capable of binding to a receptor of the one or more T-cells, and wherein the binding is insufficient to activate the one or more T-cells. In some embodiments, the peptide binds to one or more HLA binding groove domain residues of the human HLA class 1 heavy chain sequence. In some embodiments, the first peptide modulates a conformation of the human HLA class 1 heavy chain sequence. In some embodiments, the conformation prevents the one or more T-cells from binding the human HLA class 1 heavy chain sequence. In some embodiments, the peptide comprises greater than or equal to 8, 9, 10, 11, 12, 13, or 14 amino acids. In some embodiments, the nucleic acid molecule further comprises one or more sequences encoding one or more linkers between the sequence encoding the peptide and the sequence encoding the human HLA class 1 heavy chain sequence. In some embodiments, the one or more linkers are configured to resist proteolytic cleavage. In some embodiments, the one or more linkers comprise a conformation configured to not block one or more killer-cell immunoglobulin-like receptor (KIR) binding sites on the human HLA class 1 heavy chain sequence. In some embodiments, a sequence of the one or more sequences encoding one or more linkers is displaced between the sequence encoding the peptide and the sequence encoding the human β2-microglobulin peptide, between the sequence encoding the human β2-microglobulin peptide and the sequence encoding the human HLA class 1 heavy chain sequence, or both. In some embodiments, a linker of the one or more linkers comprises a sequence at least about 70%, 80%, 90%, or 99% identical to any one of SEQ ID NOs: 48-54. In some embodiments, the nucleic acid molecule further comprises a sequence encoding one or more immune checkpoint agonists. In some embodiments, the one or more immune checkpoint agonists comprise CD47, PD-L1, A2AR, B7-H3, B7-H4, BTLA, CTLA-4, IDO, KIR, LAG3, NOX2, PD-1, TIM-3, VISTA, SIGLEC7, or combination thereof. In some embodiments, the nucleic acid molecule further comprises a sequence encoding one or more knocked out proteins corresponding to a receptor of the one or more immune checkpoint agonists. In some embodiments, the sequence encoding the human HLA class 1 heavy chain sequence comprises an HLA-E sequence or a fragment thereof, an HLA-F sequence or a fragment thereof, an HLA-G sequence or a fragment thereof, or any combination thereof. In some embodiments, at least one of the HLA-E sequence or the fragment thereof, HLA-F sequence or the fragment thereof, HLA-G sequence or the fragment thereof, or any combination thereof is inhibited from eliciting a T-cell response when the human HLA class 1 heavy chain sequence is interrogated by one or more T-cells. In some embodiments, the nucleic acid molecule further comprises a sequence encoding one or more knocked out proteins corresponding class II, major histocompatibility complex, transactivator (CIITA). In some embodiments, the nucleic acid molecule further comprises a sequence encoding a regulatory peptide. In some embodiments, the regulatory peptide is an apoptosis-inducing peptide. In some embodiments, the nucleic acid molecule further comprises a sequence encoding an epitope configured to allow for detection of the complex. In some embodiments, the epitope comprises 3,5-dinitrosalicylic acid. In some embodiments, the nucleic acid molecule further comprises a sequence encoding one or more knocked out proteins. In some embodiments, the one or more knocked out proteins are selected from blood group A antigen and blood group B antigen. In some embodiments, the nucleic acid molecule comprises, a, the sequence encoding the peptide; b. a first sequence encoding a first linker of the one or more sequences encoding one or more linkers; c. the sequence encoding the human β2-microglobulin peptide; d. a second sequence encoding a second linker of the one or more sequences encoding one or more linkers; and e. the sequence encoding the human HLA class 1 heavy chain sequence.

Provided herein, in another aspect, is a nucleic acid molecule comprising a sequence encoding a complex comprising one or more Class 1 human leukocyte antigen (HLA) proteins, wherein the one or more Class 1 HLA proteins are inhibited from eliciting a T-cell response when the complex is interrogated by one or more T-cells, and wherein the nucleic acid molecule comprises,
a. a sequence encoding a peptide, wherein the peptide is incapable of activating the one or more T-cells;
b. a first sequence encoding a first linker; and
c. a sequence encoding one or more Class 1 HLA proteins;
   wherein the first linker comprises a conformation configured to not block one or more killer-cell immunoglobulin-like receptor (KIR) binding sites on the human HLA class 1 heavy chain sequence, and wherein the conformation is further configured to resist proteolytic cleavage. In some embodiments, the nucleic acid molecule further comprises a sequence encoding a human β2-microglobulin peptide between the sequence encoding the linker and the sequence encoding the human HLA class 1 heavy chain sequence. In some embodiments, the nucleic acid molecule further comprises an additional sequence encoding an additional linker between the sequence encoding the human β2-microglobulin peptide and the sequence encoding the human HLA class 1 heavy chain sequence, wherein the additional linker comprises a conformation configured to resist proteolytic cleavage. In some embodiments, the additional linker is further configured to not block one or more killer-cell immunoglobulin-like receptor (KIR) binding sites on the human HLA class 1 heavy chain sequence. In some embodiments, the first linker comprises a sequence at least about 70%, 80%, 90%, or 99% identical to any one of SEQ ID NOs: 48-54 or the additional linker comprises a sequence at least about 70%, 80%, 90%, or 99% identical to any one of SEQ ID NOs: 48-54. In some embodiments, the sequence encoding a human HLA class 1 heavy chain sequence comprises one or more mutations, wherein the one or more mutations inhibit the human HLA class 1 heavy chain sequence from eliciting a T-cell response when the human HLA class 1 heavy chain sequence is interrogated by one or more CD8 cells. In some embodiments, the one or more mutations comprises a mutation of one or more of amino acid residues 115, 122, 128, 194, 197, 198, 212, 214, 222, 223, 224, 225, 226, 227, 228, 229, 230, 231, 232, 233, 243, 245, 248, 262, or any combination thereof. In some embodiments, the nucleic acid molecule further comprises a sequence encoding one or more proteins or fragments thereof that inhibit an immune response by the complement system. In some embodiments, the one or more proteins or fragments thereof are selected from CD48, CD59, or a combination thereof.

Provided herein, in another aspect, is a method for generating a nucleic acid molecule as described herein, comprising displacing a sequence encoding a region configured to receive a sequence comprising the deletion in the HLA locus, a sequence encoding the human HLA class 1 heavy chain sequence, or any combination thereof.

Provided herein, in another aspect, is a method of generating an immune incompetent cell, comprising administering a complex or a nucleic acid as described herein to a cell.

In some embodiments, the nucleic acid is delivered to the cell's genome. In some embodiments, cell is incubated with the complex. In some embodiments, the cell is a stem cell. In some embodiments, the stem cell is an Induced Pluripotent stem cell (iPSC).

Provided herein, in another aspect, is a method of treating a disease or disorder in a subject in need thereof, comprising administering a therapeutically effective amount of a nucleic acid molecule as described here or an immune incompetent cell as described herein to the subject.

In some embodiments, the disease is an autoimmune disease. In some embodiments, the disease is a cancer. In some embodiments, the disease is a degenerative disease.

Provided herein, in another aspect, is a method of inhibiting a human leukocyte antigen (HLA) comprising contacting the HLA with a peptide that does not comprise T-cell receptor-binding residues or fragments.

In some embodiments, the peptide binds to one or more HLA binding groove domain residues of the HLA. In some embodiments, the peptide modulates a conformation of the HLA. In some embodiments, the conformation prevents a T-cell from binding the HLA. In some embodiments, the peptide comprises greater than or equal to 8, 9, 10, 11, 12, 13, or 14 amino acids. In some embodiments, the HLA is synthetic.

### INCORPORATION BY REFERENCE

All publications, patents, and patent applications mentioned in this specification and appendences attached hereto, are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “Figure” and “FIG.” herein), of which:

FIG. 1 shows a domain view of the synthetic human leukocyte antigen (synHLA) complex provided herein.

FIG. 2 shows a three-dimensional view of the construct architecture of the synthetic human leukocyte antigen (synHLA) complex provided herein.

FIG. 3 shows an HLA-bound immunogenic peptide engaging with a T-cell receptor, resulting in T-cell activation.

FIG. 4 shows the synthetic human leukocyte antigen (synHLA) complex provided herein engaging with a T-cell receptor, resulting in failed T-cell activation.

FIG. 5 shows a single-chain trimer (SCT) in complex with killer-cell immunoglobulin-like receptor (KIR), resulting in a blocked MR interaction and a “missing self” immune signal.

FIG. 6 shows the synthetic human leukocyte antigen (synHLA) complex provided herein in complex with killer-cell immunoglobulin-like receptor (MR), resulting in a successful MR interaction and no “missing self” immune signal.

FIG. 7 shows an overlay of a single-chain trimer (SCT) in complex with killer-cell immunoglobulin-like receptor (KIR) and the synthetic human leukocyte antigen (synHLA) complex provided herein in complex with killer-cell immunoglobulin-like receptor (KIR).

FIG. 8 shows the synthetic human leukocyte antigen (synHLA) complex provided herein engaging with CD8.

FIG. 9 shows the immune incompetent cell provided herein.

FIG. 10 shows an SDS-PAGE gel of synHLA complexes as described herein expressed recombinantly in bacteria.

FIG. 11 shows an SDS-PAGE of a synHLA complex as described herein expressed recombinantly in bacteria.

FIG. 12A shows a raw thermal melt curve of SYNC4-1 and SYNC4-1+KIR2DL2.

FIG. 12B shows the first derivative of a thermal melt curve of SYNC4-1 and SYNC4-1+KIR2DL2.

FIG. 13A shows a raw thermal melt curve of SYNC4-1, SYNC4-1+KIR2DL2, and KIR2DL2.

FIG. 13B shows the first derivative of a thermal melt curve of SYNC4-1, SYNC4-1+KIR2DL2, and KIR2DL2.

FIG. 14A shows a raw thermal melt curve of SYNA1-1, SYNA1-1+KIR2DL2, and KIR2DL2.

FIG. 14B shows the first derivative of a thermal melt curve of SYNA1-1, SYNA1-1+KIR2DL2, and KIR2DL2.

FIG. 15 shows a domain view of the synthetic human leukocyte antigen (synHLA) complex provided herein as designed for expression in bacteria.

### DETAILED DESCRIPTION OF THE INVENTION

### Definitions

Whenever the term “at least,” “greater than,” or “greater than or equal to” precedes the first numerical value in a series of two or more numerical values, the term “at least,” “greater than” or “greater than or equal to” applies to each of the numerical values in that series of numerical values. For example, greater than or equal to 1, 2, or 3 is equivalent to greater than or equal to 1, greater than or equal to 2, or greater than or equal to 3.

Whenever the term “no more than,” “less than,” or “less than or equal to” precedes the first numerical value in a series of two or more numerical values, the term “no more than,” “less than,” or “less than or equal to” applies to each of the numerical values in that series of numerical values. For example, less than or equal to 3, 2, or 1 is equivalent to less than or equal to 3, less than or equal to 2, or less than or equal to 1.

As used herein, a “T-cell” is a cell comprising a T-cell receptor.

As used herein, a “peptide” is a chain of between two and fifty amino acid residues.

A “pharmaceutically acceptable carrier” refers to an ingredient in a pharmaceutical formulation, other than an active ingredient, which is nontoxic to a subject. A pharmaceutically acceptable carrier includes, but is not limited to, a buffer, excipient, stabilizer, or preservative, such as those known in the art, for example, described in Remington's Pharmaceutical Sciences 16th edition, Osol, A. Ed. (1980).

As used herein, “treatment” or “treating” is an approach for obtaining beneficial or desired results including and preferably clinical results. For example, beneficial or desired clinical results include, but are not limited to, one or more of the following: decreasing symptoms resulting from the disease, increasing the quality of life of those suffering from the disease, decreasing the dose of other medications required to treat the disease, delaying the progression of the disease, and/or prolonging survival of individuals.

As used herein, an “effective dosage” or “effective amount” of complex, nucleic acid molecule, immune incompetent cell, or pharmaceutical composition thereof is an amount sufficient to effect beneficial or desired results. For prophylactic use, beneficial or desired results include results such as eliminating or reducing the risk, lessening the severity, or delaying the onset of the disease, including biochemical, histological and/or behavioral symptoms of the disease, its complications and intermediate pathological phenotypes presenting during development of the disease. For therapeutic use, beneficial or desired results include clinical results such as decreasing one or more symptoms resulting from the disease, increasing the quality of life of those suffering from the disease, decreasing the dose of other medications required to treat the disease, enhancing effect of another medication such as via targeting, delaying the progression of the disease, and/or prolonging survival. In the case of cancer or tumor, an effective amount of the drug may have the effect in reducing the number of cancer cells; reducing the tumor size; inhibiting (i.e., slow to some extent and preferably stop) cancer cell infiltration into peripheral organs; inhibit (i.e., slow to some extent and preferably stop) tumor metastasis; inhibiting, to some extent, tumor growth; and/or relieving to some extent one or more of the symptoms associated with the disorder. An effective dosage can be administered in one or more administrations. For purposes of this invention, an effective dosage of complex, nucleic acid molecule, immune incompetent cell, or pharmaceutical composition thereof is an amount sufficient to accomplish prophylactic or therapeutic treatment either directly or indirectly. As is understood in the clinical context, an effective dosage of a complex, nucleic acid molecule, immune incompetent cell, or pharmaceutical composition thereof may or may not be achieved in conjunction with another complex, nucleic acid molecule, immune incompetent cell, or pharmaceutical composition thereof. Thus, an “effective dosage” may be considered in the context of administering one or more therapeutic agents, and a single agent may be considered to be given in an effective amount if, in conjunction with one or more other agents, a desirable result may be or is achieved.

As defined herein, the term “inhibition”, “inhibit”, “inhibiting” and the like in reference to a protein-inhibitor interaction means negatively affecting (e.g. decreasing) the activity or function of the protein relative to the activity or function of the protein in the absence of the inhibitor. Inhibition may refer to reduction of a disease or symptoms of disease. Inhibition may refer to a reduction in the activity of a particular protein or nucleic acid target. The protein may be deoxycytidine kinase. Thus, inhibition includes, at least in part, partially or totally blocking stimulation, decreasing, preventing, or delaying activation, or inactivating, desensitizing, or down-regulating signal transduction or enzymatic activity or the amount of a protein.

The term “modulator” refers to a composition that increases or decreases the level of a target molecule or the function of a target molecule or the physical state of the target of the molecule.

The term “modulate” is used in accordance with its plain ordinary meaning and refers to the act of changing or varying one or more properties. “Modulation” refers to the process of changing or varying one or more properties. For example, a modulator of a target protein changes by increasing or decreasing a property or function of the target molecule or the amount of the target molecule. A modulator of a disease decreases a symptom, cause, or characteristic of the targeted disease.

“Pharmaceutically acceptable excipient” and “pharmaceutically acceptable carrier” refer to a substance that aids the administration of an active agent to and absorption by a subject and can be included in the compositions of the present invention without causing a significant adverse toxicological effect on the patient. Non-limiting examples of pharmaceutically acceptable excipients include water, NaCl, normal saline solutions, lactated Ringer's, normal sucrose, normal glucose, binders, fillers, disintegrants, lubricants, coatings, sweeteners, flavors, salt solutions (such as Ringer's solution), alcohols, oils, gelatins, carbohydrates such as lactose, amylose or starch, fatty acid esters, hydroxymethycellulose, polyvinyl pyrrolidine, and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with the complexes, nucleic acid molecules, or immune incompetent cells of the invention. One of skill in the art will recognize that other pharmaceutical excipients are useful in the present invention.

As used herein, the term “administering” means oral administration, administration as a suppository, topical contact, intravenous, parenteral, intraperitoneal, intramuscular, intralesional, intrathecal, intranasal or subcutaneous administration, or the implantation of a slow-release device, e.g., a mini-osmotic pump, to a subject. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc.

“Patient,” “subject,” “patient in need thereof,” and “subject in need thereof” are herein used interchangeably and refer to a living organism suffering from or prone to a disease or condition that can be treated by administration of a pharmaceutical composition as provided herein. Non-limiting examples include humans, other mammals, bovines, rats, mice, dogs, monkeys, goat, sheep, cows, deer, and other non-mammalian animals. In some embodiments, a patient is human. A “cancer-patient” is a patient suffering from, or prone to developing cancer.

Unless clearly indicated otherwise, the term “individual” as used herein refers to a mammal, including but not limited to, bovine, horse, feline, rabbit, canine, rodent, or primate (e.g., human). In some embodiments, an individual is a human. In some embodiments, an individual is a non-human primate such as chimpanzees and other apes and monkey species. In some embodiments, an individual is a farm animal such as cattle, horses, sheep, goats and swine; pets such as rabbits, dogs and cats; laboratory animals including rodents, such as rats, mice, and guinea pigs; and the like. In some embodiments, the invention find use in both human medicine and in the veterinary context.

“Disease” or “condition” refer to a state of being or health status of a patient or subject capable of being treated with the complexes, nucleic acid molecules, immune incompetent cells, or methods provided herein. In some embodiments, the disease as used herein refers to cancer.

As used herein, “immune checkpoint agonist” refers to an agent which results in the activation of one or more immune checkpoint proteins. For example, immune checkpoint agonists include, but are not limited to, CD47, PD-L1, A2AR, B7-H3, B7-H4, BTLA, CTLA-4, IDO, KIR, LAG3, NOX2, PD-1, TIM-3, VISTA, and SIGLEC7.

As used herein, “mutation” refers to an alteration in the sequence of a nucleic acid molecule. Mutations include, but are not limited to, insertions, deletions, and substitutions.

As used herein, the abbreviations for amino acids are conventional and can be as follows: alanine (A, Ala); arginine (R, Arg); asparagine (N, Asn); aspartic acid (D, Asp); cysteine (C, Cys); glutamic acid (E, Glu); glutamine (Q, Gln); glycine (G, Gly); histidine (H, His); isoleucine (I, Ile); leucine (L, Leu); lysine (K, Lys); methionine (M, Met); phenylalanine (F, Phe); proline (P, Pro); serine (S, Ser); threonine (T, Thr); tryptophan (W, Trp); tyrosine (Y, Tyr); valine (V, Val). Other amino acids include citrulline (Cit); homocysteine (Hey); hydroxyproline (Hyp); ornithine (Orn); and thyroxine (Thx). Examples of amino acids that are not charged at physiological pH include, but are not limited to, alanine, asparagine, cysteine, glutamine, glycine, isoleucine, leucine, methionine, phenylalanine, proline, serine, threonine, tryptophan, tyrosine, and valine.

As used herein, an “anchor residue” of a peptide is a conserved amino acid residue that plays a role in binding the peptide into the groove of a given HLA allele.

As used herein and in the appended claims, the singular forms “a,” “an,” and “the” include plural reference unless the context clearly indicates otherwise.

It is understood that aspect and variations of the invention described herein include “consisting” and/or “consisting essentially of” aspects and variations.

Synthetic Human Leukocyte Antigen (synHLA) Complex

Provided herein, in one aspect, is a complex comprising one or more human leukocyte antigens (HLAs), wherein the one or more HLAs are inhibited from eliciting a T-cell response when the complex is interrogated by one or more T-cells.

In some embodiments, the complex comprises, in N-terminus to C-terminus order, a segment comprising a peptide and a segment comprising a human HLA class 1 heavy chain sequence.

In some embodiments, the human HLA class 1 heavy chain sequence comprises one or more class 1 HLAs. In some embodiments, the human HLA class 1 heavy chain sequence comprises HLA-A, HLA-B, HLA-C, or any combination thereof. In some embodiments, the human HLA class 1 heavy chain sequence comprises HLA-A. In some embodiments, the human HLA class 1 heavy chain sequence comprises HLA-B. In some embodiments, the human HLA class 1 heavy chain sequence comprises HLA-C. In some embodiments, the human HLA class 1 heavy chain sequence comprises HLA-A and HLA-B. In some embodiments, the human HLA class 1 heavy chain sequence comprises HLA-A and HLA-C. In some embodiments, the human HLA class 1 heavy chain sequence comprises HLA-B and HLA-C. In some embodiments, the human HLA class 1 heavy chain sequence comprises HLA-A, HLA-B, and HLA-C. A In some embodiments, the human HLA class 1 heavy chain sequence comprise multiple versions of HLA-A, HLA-B, HLA-C, or any combination thereof. In some embodiments, the human HLA class 1 heavy chain sequence comprise multiple versions of HLA-A. In some embodiments, the human HLA class 1 heavy chain sequence comprise multiple versions of HLA-B. In some embodiments, the human HLA class 1 heavy chain sequence comprise multiple versions of HLA-C. In some embodiments, the human HLA class 1 heavy chain sequence comprise multiple versions of HLA-A and HLA-B. In some embodiments, the human HLA class 1 heavy chain sequence comprise multiple versions of HLA-A and HLA-C. In some embodiments, the human HLA class 1 heavy chain sequence comprise multiple versions of HLA-B and HLA-C. In some embodiments, the human HLA class 1 heavy chain sequence comprise multiple versions of HLA-A, HLA-B, and HLA-C. In some embodiments, the human HLA class 1 heavy chain sequence comprises the HLA-A, wherein the HLA-A is displaced between the HLA-B and the HLA-C.

In some embodiments, the complex further comprises one or more immune checkpoint agonists. In some embodiments, the one or more immune checkpoint agonists comprise CD47, PD-L1, A2AR, B7-H3, B7-H4, BTLA, CTLA-4, IDO, KIR, LAG3, NOX2, PD-1, TIM-3, VISTA, SIGLEC7, or combination thereof. In some embodiments, the complex comprises CD47. In some embodiments, the complex comprises PD-L1. In some embodiments, the complex comprises A2AR. In some embodiments, the complex comprises B7-H3. In some embodiments, the complex comprises B7-H4. In some embodiments, the complex comprises BTLA. In some embodiments, the complex comprises CTLA-4. In some embodiments, the complex comprises IDO. In some embodiments, the complex comprises MR. In some embodiments, the complex comprises LAG3. In some embodiments, the complex comprises NOX2. In some embodiments, the complex comprises PD-1. In some embodiments, the complex comprises TIM-3. In some embodiments, the complex comprises VISTA. In some embodiments, the complex comprises SIGLEC7.

In some embodiments, the human HLA class 1 heavy chain sequence comprises HLA-E or a fragment thereof, HLA-F or a fragment thereof, HLA-G or a fragment thereof, or any combination thereof. In some embodiments, the human HLA class 1 heavy chain sequence comprises HLA-E or a fragment thereof. In some embodiments, the human HLA class 1 heavy chain sequence comprises HLA-F or a fragment thereof. In some embodiments, the human HLA class 1 heavy chain sequence comprises HLA-G or a fragment thereof. In some embodiments, the human HLA class 1 heavy chain sequence comprises HLA-E or a fragment thereof and HLA-F or a fragment thereof. In some embodiments, the human HLA class 1 heavy chain sequence comprises HLA-E or a fragment thereof and HLA-G or a fragment thereof. In some embodiments, the human HLA class 1 heavy chain sequence comprises HLA-F or a fragment thereof and HLA-G or a fragment thereof. In some embodiments, the human HLA class 1 heavy chain sequence comprises HLA-E or a fragment thereof, HLA-F or a fragment thereof, and HLA-G or a fragment thereof. In some embodiments, at least one of the HLA-E or the fragment thereof, HLA-F or the fragment thereof, HLA-G or the fragment thereof, or any combination thereof is inhibited from eliciting a T-cell response when the complex is interrogated by one or more T-cells. In some embodiments, HLA-E or the fragment thereof is inhibited from eliciting a T-cell response when the complex is interrogated by one or more T-cells. In some embodiments, HLA-F or the fragment thereof is inhibited from eliciting a T-cell response when the complex is interrogated by one or more T-cells. In some embodiments, HLA-G or the fragment thereof is inhibited from eliciting a T-cell response when the complex is interrogated by one or more T-cells. In some embodiments, HLA-E or the fragment thereof and HLA-F or the fragment thereof are inhibited from eliciting a T-cell response when the complex is interrogated by one or more T-cells. In some embodiments, HLA-E or the fragment thereof and HLA-G or the fragment thereof are inhibited from eliciting a T-cell response when the complex is interrogated by one or more T-cells. In some embodiments, HLA-F or the fragment thereof and HLA-G or the fragment thereof are inhibited from eliciting a T-cell response when the complex is interrogated by one or more T-cells. In some embodiments, HLA-E or the fragment thereof, HLA-F or the fragment thereof, and HLA-G or the fragment thereof are inhibited from eliciting a T-cell response when the complex is interrogated by one or more T-cells.

In some embodiments, the complex further comprises an epitope configured to allow for detection of the complex. In some embodiments, the epitope comprises 3,5-dinitrosalicylic acid.

In some embodiments, the complex comprises a human β2-microglobulin sequence. In some embodiments, the human β2-microglobulin sequence is a wild-type human β2-microglobulin sequence.

In some embodiments, the complex comprises, in N-terminus to C-terminus order,
a. the peptide;
b. a first linker of the one or more linkers;
c. the human β2-microglobulin sequence;
d. a second linker of the one or more linkers; and
e. the human HLA class 1 heavy chain sequence.

Provided herein, in another aspect, is a complex comprising one or more human leukocyte antigens (HLAs), wherein the one or more HLAs are inhibited from eliciting a T-cell response when the complex is interrogated by one or more T-cells, and wherein the complex comprises, in N-terminus to C-terminus order,
a. a peptide, wherein the peptide is incapable of activating the one or more T-cells;
b. a first linker; and
c. a segment comprising a human HLA class 1 heavy chain sequence;
   wherein the first linker comprises a conformation configured to not block one or more killer-cell immunoglobulin-like receptor (KIR) binding sites on the human HLA class 1 heavy chain sequence, and wherein the conformation is further configured to resist proteolytic cleavage.

In some embodiments, the complex comprises a sequence at least about 70%, about 80%, about 85%, about 86%, about 87%, about 88%, about 89%, about 90%, about 91%, about 92%, about 93%, about 94%, about 95%, about 96%, about 97%, about 98%, about 99%, about 99.9%, or about 100% identical to a sequence listed in Table A below.

TABLE A
HLA complex sequences

| | SEQ | |
|---|---|---|
| | ID | |
| Description | NO: | Sequence |
| Single chain | 1 | GILGFVFTLGGGGSGGGGSGGGGSI |
| trimer | | QRTPKIQVYSRHPAENGKSNFLNCY |
| | | VSGFHPSDIEVDLLKNGERIEKVEH |
| | | SDLSFSKDWSFYLLYYTEFTPTEKD |
| | | EYACRVNHVTLSQPKIVKWDRDMGG |
| | | GGSGGGGSGGGGSGSHSMRYFFTSV |
| | | SRPGRGEPRFIAVGYVDDTQFVRFD |
| | | SDAASQRMEPRAPWIEQEGPEYWDG |
| | | ETRKVKAHSQTHRVDLGTLRGYYNQ |
| | | SEAGSHTVQRMYGCDVGSDWRFLRG |
| | | YHQYAYDGKDYIALKEDLRSWTAAD |
| | | MAAQTTKHKWEAAHVAEQLRAYLEG |
| | | TCVEWLRRYLENGKETLQRTDAPKT |
| | | HMTHHAVSDHEATLRCWALSFYPAE |
| | | ITLTWQRDGEDQTQDTELVETRPAG |
| | | DGTFQKWAAVVVPSGQEQRYTCHVQ |
| | | HEGLPKPLTLRWEENLYFQGHHHHH |
| | | H |
| Single chain | 2 | MSRSVALAVLALLSLSGLEAGILGF |
| trimer | | VFTLGGGGSGGGGSGGGGSIQRTPK |
| | | IQVYSRHPAENGKSNFLNCYVSGFH |
| | | PSDIEVDLLKNGERIEKVEHSDLSF |
| | | SKDWSFYLLYYTEFTPTEKDEYACR |
| | | VNHVTLSQPKIVKWDRDMGGGGSGG |
| | | GGSGGGGSGGGGSGSHSMRYFFTSV |
| | | SRPGRGEPRFIAVGYVDDTQFVRFD |
| | | SDAASQRMEPRAPWIEQEGPEYWDG |
| | | ETRKVKAHSQTHRVDLGTLRGYYNQ |
| | | SEAGSHTVQRMYGCDVGSDWRFLRG |
| | | YHQYAYDGKDYIALKEDLRSWTAAD |
| | | MAAQTTKHKWEAAHVAEQLRAYLEG |
| | | TCVEWLRRYLENGKETLQRTDAPKT |
| | | HMTHHAVSDHEATLRCWALSFYPAE |
| | | ITLTWQRDGEDQTQDTELVETRPAG |
| | | DGTFQKWAAVVVPSGQEQRYTCHVQ |
| | | HEGLPKPLTLRWEPSSQPTIPIVGI |
| | | IAGLVLFGAVITGAVVAAVMWRRKS |
| | | SDRKGGSYSQAASSDSAQGSDVSLT |
| | | ACKV |
| Single chain | 3 | GILGFVFTLGGGGSGGGGSGGGGSI |
| trimer | | QRTPKIQVYSRHPAENGKSNFLNCY |
| | | VSGFHPSDIEVDLLKNGERIEKVEH |
| | | SDLSFSKDWSFYLLYYTEFTPTEKD |
| | | EYACRVNHVTLSQPKIVKWDRDMGG |
| | | GGSGGGGSGGGGSGGGGSGSHSMRY |
| | | FFTSVSRPGRGEPRFIAVGYVDDTQ |
| | | FVRFDSDAASQRMEPRAPWIEQEGP |
| | | EYWDGETRKVKAHSQTHRVDLGTLR |
| | | GYYNQSEAGSHTVQRMYGCDVGSDW |
| | | RFLRGYHQYAYDGKDYIALKEDLRS |
| | | WTAADMAAQTTKHKWEAAHVAEQLR |
| | | AYLEGTCVEWLRRYLENGKETLQRT |
| | | DAPKTHMTHHAVSDHEATLRCWALS |
| | | FYPAEITLTWQRDGEDQTQDTELVE |
| | | TRPAGDGTFQKWAAVVVPSGQEQRY |
| | | TCHVQHEGLPKPLTLRWEENLYFQG |
| | | HHHHHH |
| Single chain | 4 | GILGFVFTLGGGGSGGGGSGGGGSI |
| trimer | | QRTPKIQVYSRHPAENGKSNFLNCY |
| | | VSGFHPSDIEVDLLKNGERIEKVEH |
| | | SDLSFSKDWSFYLLYYTEFTPTEKD |
| | | EYACRVNHVTLSQPKIVKWDRDMGG |
| | | GGSGGGGSGGGGSGGGGSGSHSMRY |
| | | FFTSVSRPGRGEPRFIAVGYVDDTQ |
| | | FVRFDSDAASQRMEPRAPWIEQEGP |
| | | EYWDGETRKVKAHSQTHRVDLGTLR |
| | | GYYNQSEAGSHTVQRMYGCDVGSDW |
| | | RFLRGYHQYAYDGKDYIALKEDLRS |
| | | WTAADMAAQTTKHKWEAAHVAEQLR |
| | | AYLEGTCVEWLRRYLENGKETLQRT |
| | | DAPKTHMTHHAVSDHEATLRCWALS |
| | | FYPAEITLTWQRDGEDQTQDTELVE |
| | | TRPAGDGTFQKWAAVVVPSGQEQRY |
| | | TCHVQHEGLPKPLTLRWEENLYFQG |
| | | HHHHHH |
| Single chain | 5 | SYRPGTVALGGGGSGGGGSGGGGSI |
| trimer | | QRTPKIQVYSRHPAENGKSNFLNCY |
| | | VSGFHPSDIEVDLLKNGERIEKVEH |
| | | SDLSFSKDWSFYLLYYTEFTPTEKD |
| | | EYACRVNHVTLSQPKIVKWDRDMGG |
| | | GGSGGGGSGGGGSCSHSMRYFDTAV |
| | | SRPGRGEPRFISVGYVDDTQFVRFD |
| | | SDAASPRGEPRAPWVEQEGPEYWDR |
| | | ETQKYKRQAQADRVSLRNLRGYYNQ |
| | | SEDGSHTLQRMSGCDLGPDGRLLRG |
| | | YDQSAYDGKDYIALNEDLRSWTAAD |
| | | TAAQITQRKLEAARAAEQLRAYLEG |
| | | TCVEWLRRYLENGKETLQRAEPPKT |
| | | HVTHHPLSDHEATLRCWALGFYPAE |
| | | ITLTWQRDGEDQTQDTELVETRPAG |
| | | DGTFQKWAAVVVPSGQEQRYTCHMQ |
| | | HEGLQEPLTLSWEPSENLYFQGHHH |
| | | HHH |
| Single chain | 6 | SYRPGTVALGGGGSGGGGSGGGGSI |
| trimer | | QRTPKIQVYSRHPAENGKSNFLNCY |
| | | VSGFHPSDIEVDLLKNGERIEKVEH |
| | | SDLSFSKDWSFYLLYYTEFTPTEKD |
| | | EYACRVNHVTLSQPKIVKWDRDMGG |
| | | GGSGGGGSGGGGSGSHSMRYFDTAV |
| | | SRPGRGEPRFISVGYVDDTQFVRFD |
| | | SDAASPRGEPRAPWVEQEGPEYWDR |
| | | ETQKYKRQAQADRVSLRNLRGYYNQ |
| | | SEDGSHTLQRMSGCDLGPDGRLLRG |
| | | YDQSAYDGKDYIALNEDLRSWTAAD |
| | | TAAQITQRKLEAARAAEQLRAYLEG |
| | | TCVEWLRRYLENGKETLQRAEPPKT |
| | | HVTHHPLSDHEATLRCWALGFYPAE |
| | | ITLTWQRDGEDQTQDTELVETRPAG |
| | | DGTFQKWAAVVVPSGQEQRYTCHMQ |
| | | HEGLQEPLTLSWEPSENLYFQGHHH |
| | | HHH |
| Single chain | 7 | MSRSVALAVLALLSLSGLEAIQRTP |
| dimer | | KIQVYSRHPAENGKSNFLNCYVSGF |
| | | HPSDIEVDLLKNGERIEKVEHSDLS |
| | | FSKDWSFYLLYYTEFTPTEKDEYAC |
| | | RVNHVTLSQPKIVKWDRDMGGGGSG |
| | | GGGSGGGGSGGGGSGSHSMRYFFTS |
| | | VSRPGRGEPRFIAVGYVDDTQFVRF |
| | | DSDAASQRMEPRAPWIEQEGPEYWD |
| | | GETRKVKAHSQTHRVDLGTLRGYYN |
| | | QSEAGSHTVQRMYGCDVGSDWRFLR |
| | | GYHQYAYDGKDYIALKEDLRSWTAA |
| | | DMAAQTTKHKWEAAHVAEQLRAYLE |
| | | GTCVEWLRRYLENGKETLQRTDAPK |
| | | THMTHHAVSDHEATLRCWALSFYPA |
| | | EITLTWQRDGEDQTQDTELVETRPA |
| | | GDGTFQKWAAVVVPSGQEQRYTCHV |
| | | QHEGLPKPLTLRWEPSSQPTIPIVG |
| | | IIAGLVLFGAVITGAVVAAVMWRRK |
| | | SSDRKGGSYSQAASSDSAQGSDVSL |
| | | TACKV |
| Single chain | 8 | SYRPGTVALGGGGSGGGGSIQRTPK |
| dimer | | IQVYSRHPAENGKSNFLNCYVSGFH |
| | | PSDIEVDLLKNGERIEKVEHSDLSF |
| | | SKDWSFYLLYYTEFTPTEKDEYACR |
| | | VNHVTLSQPKIVKWDRDM |
| Single chain | 9 | SYRPGTVALGCGGSGGGGSIQRTPK |
| dimer | | IQVYSRHPAENGKSNFLNCYVSGFH |
| | | PSDIEVDLLKNGERIEKVEHSDLSF |
| | | SKDWSFYLLYYTEFTPTEKDEYACR |
| | | VNHVTLSQPKIVKWDRDM |
| Single chain | 10 | SYRPGTVALGGGGSGGGGSGSIQRT |
| dimer | | PKIQVYSRHPAENGKSNFLNCYVSG |
| | | FHPSDIEVDLLKNGERIEKVEHSDL |
| | | SFSKDWSFYLLYYTEFTPTEKDEYA |
| | | CRVNHVTLSQPKIVKWDRDM |
| Single chain | 11 | SYRPGTVALGCGGSGGGGSGSIQRT |
| dimer | | PKIQVYSRHPAENGKSNFLNCYVSG |
| | | FHPSDIEVDLLKNGERIEKVEHSDL |
| | | SFSKDWSFYLLYYTEFTPTEKDEYA |
| | | CRVNHVTLSQPKIVKWDRDM |
| Single chain | 12 | SYRPGTVALGGGGSGGGGSGGGGSI |
| dimer | | QRTPKIQVYSRHPAENGKSNFLNCY |
| | | VSGFHPSDIEVDLLKNGERIEKVEH |
| | | SDLSFSKDWSFYLLYYTEFTPTEKD |
| | | EYACRVNHVTLSQPKIVKWDRDM |
| Single chain | 13 | SYRPGTVALGCGGSGGGGSGGGGSI |
| dimer | | QRTPKIQVYSRHPAENGKSNFLNCY |
| | | VSGFHPSDIEVDLLKNGERIEKVEH |
| | | SDLSFSKDWSFYLLYYTEFTPTEKD |
| | | EYACRVNHVTLSQPKIVKWDRDM |
| Synthetic | 14 | GILGFVFTLGGGGSGGGGSGGGGSI |
| HLA | | QRTPKIQVYSRHPAENGKSNFLNCY |
| | | VSGFHPSDIEVDLLKNGERIEKVEH |
| | | SDLSFSKDWSFYLLYYTEFTPTEKD |
| | | EYACRVNHVTLSQPKIVKWDRDMGG |
| | | GGSGGGGSGGGGSGSHSMRYFFTSV |
| | | SRPGRGEPRFIAVGYVDDTQFVRFD |
| | | SDAASQRMEPRAPWIEQEGPEYWDG |
| | | ETRKVKAHSQTHRVDLGTLRGAYNQ |
| | | SEAGSHTVQRMYGCDVGSDWRFLRG |
| | | YHQYAYDGKDYIALKEDLRSWTAAD |
| | | MAAQTTKHKWEAAHVAEQLRAYLEG |
| | | TCVEWLRRYLENGKETLQRTDAPKT |
| | | HMTHHAVSDHEATLRCWALSFYPAE |
| | | ITLTWQRDGEDQTQDTELVETRPAG |
| | | DGTFQKWAAVVVPSGQEQRYTCHVQ |
| | | HEGLPKPLTLRWEENLYFQGHHHHH |
| | | H |
| Synthetic | 15 | RYRPGTVALGGGGSGGGGSGGGGSI |
| HLA | | QRTPKIQVYSRHPAENGKSNFLNCY |
| | | VSGFHPSDIEVDLLKNGERIEKVEH |
| | | SDLSFSKDWSFYLLYYTEFTPTEKD |
| | | EYACRVNHVTLSQPKIVKWDRDMGG |
| | | GGSGGGGSGGGGSCSHSMRYFDTAV |
| | | SRPGRGEPRFISVGYVDDTQFVRFD |
| | | SDAASPRGEPRAPWVEQEGPEYWDR |
| | | ETQKYKRQAQADRVSLRNLRGAYNQ |
| | | SEDGSHTLQRMSGCDLGPDGRLLRG |
| | | YDQSAYDGKDYIALNEDLRSWTAAD |
| | | TAAQITQRKLEAARAAEQLRAYLEG |
| | | TCVEWLRRYLENGKETLQRAEPPKT |
| | | HVTHHPLSDHEATLRCWALGFYPAE |
| | | ITLTWQRDGEDQTQDTELVETRPAG |
| | | DGTFQKWAAVVVPSGQEQRYTCHMQ |
| | | HEGLQEPLTLSWEPSENLYFQGHHH |
| | | HHH |
| Synthetic | 16 | RYRPGTVALGGGGSGGGGSGGGGSI |
| HLA | | QRTPKIQVYSRHPAENGKSNFLNCY |
| | | VSGFHPSDIEVDLLKNGERIEKVEH |
| | | SDLSFSKDWSFYLLYYTEFTPTEKD |
| | | EYACRVNHVTLSQPKIVKWDRDMGG |
| | | GGSGGGGSGGGGSCSHSMRYFDTAV |
| | | SRPGRGEPRFISVGYVDDTQFVRFD |
| | | SDAASPRGEPRAPWVEQEGPEYWDR |
| | | ETQKYKRQAQADRVSLRNLRGAYNQ |
| | | SEDGSHTLQRMSGCDLGPDGRLLRG |
| | | YDQSAYDGKDYIALNEDLRSWTAAD |
| | | TAAQITQRKLEAARAAEQLRAYLEG |
| | | TCVEWLRRYLENGKETLQRAEPPKT |
| | | HVTHHPLSDHEATLRCWALGFYPAE |
| | | ITLTWQRDGEDQTQDTELVETRPAG |
| | | DGTFQKWAAVVVPSGQEQRYTCHMQ |
| | | HEGLQEPLTLSWEPSENLYFQGHHH |
| | | HHH |
| Synthetic | 17 | RYRPGTVALGGGGSGGGGSGGGGSI |
| HLA | | QRTPKIQVYSRHPAENGKSNFLNCY |
| | | VSGFHPSDIEVDLLKNGERIEKVEH |
| | | SDLSFSKDWSFYLLYYTEFTPTEKD |
| | | EYACRVNHVTLSQPKIVKWDRDMGG |
| | | GGSGGGGSGGGGSCSHSMRYFDTAV |
| | | SRPGRGEPRFISVGYVDDTQFVRFD |
| | | SDAASPRGEPRAPWVEQEGPEYWDR |
| | | ETQKYKRQAQADRVSLRNLRGAYNQ |
| | | SEDGSHTLQRMSGCDLGPDGRLLRG |
| | | YDQSAYDGKDYIALNEDLRSWTAAD |
| | | TAAQITQRKLEAARAAEQLRAYLEG |
| | | TCVEWLRRYLENGKETLQRAEPPKT |
| | | HVTHHPLSDHEATLRCWALGFYPAE |
| | | ITLTWQRDGEDQTQDTELVETRPAG |
| | | DGTFQKWAAVVVPSGQEQRYTCHMQ |
| | | HEGLQEPLTLSWEPSENLYFQGHHH |
| | | HHH |
| Synthetic | 18 | RYRPGTVALGGGGSGGGGSGSIQRT |
| HLA | | PKIQVYSRHPAENGKSNFLNCYVSG |
| | | FHPSDIEVDLLKNGERIEKVEHSDL |
| | | SFSKDWSFYLLYYTEFTPTEKDEYA |
| | | CRVNHVTLSQPKIVKWDRDMGGGGS |
| | | GGGGSGGGGSCSHSMRYFDTAVSRP |
| | | GRGEPRFISVGYVDDTQFVRFDSDA |
| | | ASPRGEPRAPWVEQEGPEYWDRETQ |
| | | KYKRQAQADRVSLRNLRGAYNQSED |
| | | GSHTLQRMSGCDLGPDGRLLRGYDQ |
| | | SAYDGKDYIALNEDLRSWTAADTAA |
| | | QITQRKLEAARAAEQLRAYLEGTCV |
| | | EWLRRYLENGKETLQRAEPPKTHVT |
| | | HHPLSDHEATLRCWALGFYPAEITL |
| | | TWQRDGEDQTQDTELVETRPAGDGT |
| | | FQKWAAVVVPSGQEQRYTCHMQHEG |
| | | LQEPLTLSWEPSENLYFQGHHHHHH |
| Synthetic | 19 | RYRPGTVALGGGGSGGGGSGSIQRT |
| HLA | | PKIQVYSRHPAENGKSNFLNCYVSG |
| | | FHPSDIEVDLLKNGERIEKVEHSDL |
| | | SFSKDWSFYLLYYTEFTPTEKDEYA |
| | | CRVNHVTLSQPKIVKWDRDMGGGGS |
| | | GGGGSGGGGSCSHSMRYFDTAVSRP |
| | | GRGEPRFISVGYVDDTQFVRFDSDA |
| | | ASPRGEPRAPWVEQEGPEYWDRETQ |
| | | KYKRQAQADRVSLRNLRGAYNQSED |
| | | GSHTLQRMSGCDLGPDGRLLRGYDQ |
| | | SAYDGKDYIALNEDLRSWTAADTAA |
| | | QITQRKLEAARAAEQLRAYLEGTCV |
| | | EWLRRYLENGKETLQRAEPPKTHVT |
| | | HHPLSDHEATLRCWALGFYPAEITL |
| | | TWQRDGEDQTQDTELVETRPAGDGT |
| | | FQKWAAVVVPSGQEQRYTCHMQHEG |
| | | LQEPLTLSWEPSENLYFQGHHHHHH |
| Synthetic | 20 | RYRPGTVALGGGGSGGGGSGSIQRT |
| HLA | | PKIQVYSRHPAENGKSNFLNCYVSG |
| | | FHPSDIEVDLLKNGERIEKVEHSDL |
| | | SFSKDWSFYLLYYTEFTPTEKDEYA |
| | | CRVNHVTLSQPKIVKWDRDMGGGGS |
| | | GGGGSGGGGSCSHSMRYFDTAVSRP |
| | | GRGEPRFISVGYVDDTQFVRFDSDA |
| | | ASPRGEPRAPWVEQEGPEYWDRETQ |
| | | KYKRQAQADRVSLRNLRGAYNQSED |
| | | GSHTLQRMSGCDLGPDGRLLRGYDQ |
| | | SAYDGKDYIALNEDLRSWTAADTAA |
| | | QITQRKLEAARAAEQLRAYLEGTCV |
| | | EWLRRYLENGKETLQRAEPPKTHVT |
| | | HHPLSDHEATLRCWALGFYPAEITL |
| | | TWQRDGEDQTQDTELVETRPAGDGT |
| | | FQKWAAVVVPSGQEQRYTCHMQHEG |
| | | LQEPLTLSWEPSENLYFQGHHHHHH |
| Synthetic | 21 | RYRPGTVALGGGGSGGGGSGSIQRT |
| HLA | | PKIQVYSRHPAENGKSNFLNCYVSG |
| | | FHPSDIEVDLLKNGERIEKVEHSDL |
| | | SFSKDWSFYLLYYTEFTPTEKDEYA |
| | | CRVNHVTLSQPKIVKWDRDMGGGGS |
| | | GGGGSGGGGSCSHSMRYFDTAVSRP |
| | | GRGEPRFISVGYVDDTQFVRFDSDA |
| | | ASPRGEPRAPWVEQEGPEYWDRETQ |
| | | KYKRQAQADRVSLRNLRGAYNQSED |
| | | GSHTLQRMSGCDLGPDGRLLRGYDQ |
| | | SAYDGKDYIALNEDLRSWTAADTAA |
| | | QITQRKLEAARAAEQLRAYLEGTCV |
| | | EWLRRYLENGKETLQRAEPPKTHVT |
| | | HHPLSDHEATLRCWALGFYPAEITL |
| | | TWQRDGEDQTQDTELVETRPAGDGT |
| | | FQKWAAVVVPSGQEQRYTCHMQHEG |
| | | LQEPLTLSWEPSENLYFQGHHHHHH |
| Synthetic | 22 | RYRPGTVALGGGGSGGGGSGSIQRT |
| HLA | | PKIQVYSRHPAENGKSNFLNCYVSG |
| | | FHPSDIEVDLLKNGERIEKVEHSDL |
| | | SFSKDWSFYLLYYTEFTPTEKDEYA |
| | | CRVNHVTLSQPKIVKWDRDMGGGGS |
| | | GGGGSGGGGSCSHSMRYFDTAVSRP |
| | | GRGEPRFISVGYVDDTQFVRFDSDA |
| | | ASPRGEPRAPWVEQEGPEYWDRETQ |
| | | KYKRQAQADRVSLRNLRGAYNQSED |
| | | GSHTLQRMSGCDLGPDGRLLRGYDQ |
| | | SAYDGKDYIALNEDLRSWTAADTAA |
| | | QITQRKLEAARAAEQLRAYLEGTCV |
| | | EWLRRYLENGKETLQRAEPPKTHVT |
| | | HHPLSDHEATLRCWALGFYPAEITL |
| | | TWQRDGEDQTQDTELVETRPAGDGT |
| | | FQKWAAVVVPSGQEQRYTCHMQHEG |
| | | LQEPLTLSWEPSENLYFQGHHHHHH |
| Synthetic | 23 | RYRPGTVALGGGGSGGGGSGSIQRT |
| HLA | | PKIQVYSRHPAENGKSNFLNCYVSG |
| | | FHPSDIEVDLLKNGERIEKVEHSDL |
| | | SFSKDWSFYLLYYTEFTPTEKDEYA |
| | | CRVNHVTLSQPKIVKWDRDMGGGGS |
| | | GGGGSGGGGSCSHSMRYFDTAVSRP |
| | | GRGEPRFISVGYVDDTQFVRFDSDA |
| | | ASPRGEPRAPWVEQEGPEYWDRETQ |
| | | KYKRQAQADRVSLRNLRGAYNQSED |
| | | GSHTLQRMSGCDLGPDGRLLRGYDQ |
| | | SAYDGKDYIALNEDLRSWTAADTAA |
| | | QITQRKLEAARAAEQLRAYLEGTCV |
| | | EWLRRYLENGKETLQRAEPPKTHVT |
| | | HHPLSDHEATLRCWALGFYPAEITL |
| | | TWQRDGEDQTQDTELVETRPAGDGT |
| | | FQKWAAVVVPSGQEQRYTCHMQHEG |
| | | LQEPLTLSWEPSENLYFQGHHHHHH |
| Synthetic | 24 | SYRPGTVALGGGGSGGGGSGGGGSI |
| HLA | | QRTPKIQVYSRHPAENGKSNFLNCY |
| | | VSGFHPSDIEVDLLKNGERIEKVEH |
| | | SDLSFSKDWSFYLLYYTEFTPTEKD |
| | | EYACRVNHVTLSQPKIVKWDRDMGG |
| | | GGSGGGGSGGGGSCSHSMRYFDTAV |
| | | SRPGRGEPRFISVGYVDDTQFVRFD |
| | | SDAASPRGEPRAPWVEQEGPEYWDR |
| | | ETQKYKRQAQADRVSLRNLRGAYNQ |
| | | SEDGSHTLQRMSGCDLGPDGRLLRG |
| | | YDQSAYDGKDYIALNEDLRSWTAAD |
| | | TAAQITQRKLEAARAAEQLRAYLEG |
| | | TCVEWLRRYLENGKETLQRAEPPKT |
| | | HVTHHPLSDHEATLRCWALGFYPAE |
| | | ITLTWQRDGEDQTQDTELVETRPAG |
| | | DGTFQKWAAVVVPSGQEQRYTCHMQ |
| | | HEGLQEPLTLSWEPSENLYFQGHHH |
| | | HHH |
| Synthetic | 25 | SYRPGTVALGGGGSGGGGSGGGGSI |
| HLA | | QRTPKIQVYSRHPAENGKSNFLNCY |
| | | VSGFHPSDIEVDLLKNGERIEKVEH |
| | | SDLSFSKDWSFYLLYYTEFTPTEKD |
| | | EYACRVNHVTLSQPKIVKWDRDMGG |
| | | GGSGGGGSGGGGSCSHSMRYFDTAV |
| | | SRPGRGEPRFISVGYVDDTQFVRFD |
| | | SDAASPRGEPRAPWVEQEGPEYWDR |
| | | ETQKYKRQAQADRVSLRNLRGAYNQ |
| | | SEDGSHTLQRMSGCDLGPDGRLLRG |
| | | YDQSAYDGKDYIALNEDLRSWTAAD |
| | | TAAQITQRKLEAARAAEQLRAYLEG |
| | | TCVEWLRRYLENGKETLQRAEPPKT |
| | | HVTHHPLSDHEATLRCWALGFYPAE |
| | | ITLTWQRDGEDQTAKTELVETRPAG |
| | | DGTFQKWAAVVVPSGQEQRYTCHMQ |
| | | HEGLQEPLTLSWEPSENLYFQGHHH |
| | | HHH |
| Synthetic | 26 | SYRPGTVALGCGGSGGGGSGGGGSI |
| HLA | | QRTPKIQVYSRHPAENGKSNFLNCY |
| | | VSGFHPSDIEVDLLKNGERIEKVEH |
| | | SDLSFSKDWSFYLLYYTEFTPTEKD |
| | | EYACRVNHVTLSQPKIVKWDRDMGG |
| | | GGSGGGGSGGGGSCSHSMRYFDTAV |
| | | SRPGRGEPRFISVGYVDDTQFVRFD |
| | | SDAASPRGEPRAPWVEQEGPEYWDR |
| | | ETQKYKRQAQADRVSLRNLRGCYNQ |
| | | SEDGSHTLQRMSGCDLGPDGRLLRG |
| | | YDQSAYDGKDYIALNEDLRSWTAAD |
| | | TAAQITQRKLEAARAAEQLRAYLEG |
| | | TCVEWLRRYLENGKETLQRAEPPKT |
| | | HVTHHPLSDHEATLRCWALGFYPAE |
| | | ITLTWQRDGEDQTAKTELVETRPAG |
| | | DGTFQKWAAVVVPSGQEQRYTCHMQ |
| | | HEGLQEPLTLSWEPSENLYFQGHHH |
| | | HHH |
| Synthetic | 27 | SYRPGTVALGGGGSGGGGSGSIQRT |
| HLA | | PKIQVYSRHPAENGKSNFLNCYVSG |
| | | FHPSDIEVDLLKNGERIEKVEHSDL |
| | | SFSKDWSFYLLYYTEFTPTEKDEYA |
| | | CRVNHVTLSQPKIVKWDRDMGGGGS |
| | | GGGGSGGGGSCSHSMRYFDTAVSRP |
| | | GRGEPRFISVGYVDDTQFVRFDSDA |
| | | ASPRGEPRAPWVEQEGPEYWDRETQ |
| | | KYKRQAQADRVSLRNLRGAYNQSED |
| | | GSHTLQRMSGCDLGPDGRLLRGYDQ |
| | | SAYDGKDYIALNEDLRSWTAADTAA |
| | | QITQRKLEAARAAEQLRAYLEGTCV |
| | | EWLRRYLENGKETLQRAEPPKTHVT |
| | | HHPLSDHEATLRCWALGFYPAEITL |
| | | TWQRDGEDQTQDTELVETRPAGDGT |
| | | FQKWAAVVVPSGQEQRYTCHMQHEG |
| | | LQEPLTLSWEPSENLYFQGHHHHHH |
| Synthetic | 28 | SYRPGTVALGGGGSGGGGSGSIQRT |
| HLA | | PKIQVYSRHPAENGKSNFLNCYVSG |
| | | FHPSDIEVDLLKNGERIEKVEHSDL |
| | | SFSKDWSFYLLYYTEFTPTEKDEYA |
| | | CRVNHVTLSQPKIVKWDRDMGGGGS |
| | | GGGGSGGGGSCSHSMRYFDTAVSRP |
| | | GRGEPRFISVGYVDDTQFVRFDSDA |
| | | ASPRGEPRAPWVEQEGPEYWDRETQ |
| | | KYKRQAQADRVSLRNLRGAYNQSED |
| | | GSHTLQRMSGCDLGPDGRLLRGYDQ |
| | | SAYDGKDYIALNEDLRSWTAADTAA |
| | | QITQRKLEAARAAEQLRAYLEGTCV |
| | | EWLRRYLENGKETLQRAEPPKTHVT |
| | | HHPLSDHEATLRCWALGFYPAEITL |
| | | TWQRDGEDQTAKTELVETRPAGDGT |
| | | FQKWAAVVVPSGQEQRYTCHMQHEG |
| | | LQEPLTLSWEPSENLYFQGHHHHHH |
| Synthetic | 29 | SYRPGTVALGCGGSGGGGSGSIQRT |
| HLA | | PKIQVYSRHPAENGKSNFLNCYVSG |
| | | FHPSDIEVDLLKNGERIEKVEHSDL |
| | | SFSKDWSFYLLYYTEFTPTEKDEYA |
| | | CRVNHVTLSQPKIVKWDRDMGGGGS |
| | | GGGGSGGGGSCSHSMRYFDTAVSRP |
| | | GRGEPRFISVGYVDDTQFVRFDSDA |
| | | ASPRGEPRAPWVEQEGPEYWDRETQ |
| | | KYKRQAQADRVSLRNLRGCYNQSED |
| | | GSHTLQRMSGCDLGPDGRLLRGYDQ |
| | | SAYDGKDYIALNEDLRSWTAADTAA |
| | | QITQRKLEAARAAEQLRAYLEGTCV |
| | | EWLRRYLENGKETLQRAEPPKTHVT |
| | | HHPLSDHEATLRCWALGFYPAEITL |
| | | TWQRDGEDQTAKTELVETRPAGDGT |
| | | FQKWAAVVVPSGQEQRYTCHMQHEG |
| | | LQEPLTLSWEPSENLYFQGHHHHHH |
| Synthetic | 30 | SYRPGTVALGGGGSGGGGSIQRTPK |
| HLA | | IQVYSRHPAENGKSNFLNCYVSGFH |
| | | PSDIEVDLLKNGERIEKVEHSDLSF |
| | | SKDWSFYLLYYTEFTPTEKDEYACR |
| | | VNHVTLSQPKIVKWDRDMGGGGSGG |
| | | GGSGGGGSCSHSMRYFDTAVSRPGR |
| | | GEPRFISVGYVDDTQFVRFDSDAAS |
| | | PRGEPRAPWVEQEGPEYWDRETQKY |
| | | KRQAQADRVSLRNLRGAYNQSEDGS |
| | | HTLQRMSGCDLGPDGRLLRGYDQSA |
| | | YDGKDYIALNEDLRSWTAADTAAQI |
| | | TQRKLEAARAAEQLRAYLEGTCVEW |
| | | LRRYLENGKETLQRAEPPKTHVTHH |
| | | PLSDHEATLRCWALGFYPAEITLTW |
| | | QRDGEDQTQDTELVETRPAGDGTFQ |
| | | KWAAVVVPSGQEQRYTCHMQHEGLQ |
| | | EPLTLSWEPSENLYFQGHHHHHH |
| Synthetic | 31 | SYRPGTVALGGGGSGGGGSIQRTPK |
| HLA | | IQVYSRHPAENGKSNFLNCYVSGFH |
| | | PSDIEVDLLKNGERIEKVEHSDLSF |
| | | SKDWSFYLLYYTEFTPTEKDEYACR |
| | | VNHVTLSQPKIVKWDRDMGGGGSGG |
| | | GGSGGGGSCSHSMRYFDTAVSRPGR |
| | | GEPRFISVGYVDDTQFVRFDSDAAS |
| | | PRGEPRAPWVEQEGPEYWDRETQKY |
| | | KRQAQADRVSLRNLRGAYNQSEDGS |
| | | HTLQRMSGCDLGPDGRLLRGYDQSA |
| | | YDGKDYIALNEDLRSWTAADTAAQI |
| | | TQRKLEAARAAEQLRAYLEGTCVEW |
| | | LRRYLENGKETLQRAEPPKTHVTHH |
| | | PLSDHEATLRCWALGFYPAEITLTW |
| | | QRDGEDQTAKTELVETRPAGDGTFQ |
| | | KWAAVVVPSGQEQRYTCHMQHEGLQ |
| | | EPLTLSWEPSENLYFQGHHHHHH |
| Synthetic | 32 | SYRPGTVALGCGGSGGGGSIQRTPK |
| HLA | | IQVYSRHPAENGKSNFLNCYVSGFH |
| | | PSDIEVDLLKNGERIEKVEHSDLSF |
| | | SKDWSFYLLYYTEFTPTEKDEYACR |
| | | VNHVTLSQPKIVKWDRDMGGGGSGG |
| | | GGSGGGGSCSHSMRYFDTAVSRPGR |
| | | GEPRFISVGYVDDTQFVRFDSDAAS |
| | | PRGEPRAPWVEQEGPEYWDRETQKY |
| | | KRQAQADRVSLRNLRGCYNQSEDGS |
| | | HTLQRMSGCDLGPDGRLLRGYDQSA |
| | | YDGKDYIALNEDLRSWTAADTAAQI |
| | | TQRKLEAARAAEQLRAYLEGTCVEW |
| | | LRRYLENGKETLQRAEPPKTHVTHH |
| | | PLSDHEATLRCWALGFYPAEITLTW |
| | | QRDGEDQTAKTELVETRPAGDGTFQ |
| | | KWAAVVVPSGQEQRYTCHMQHEGLQ |
| | | EPLTLSWEPSENLYFQGHHHHHH |
| Synthetic | 33 | SYRPGTVALGGGGSGGGGSGGGGSI |
| HLA | | QRTPKIQVYSRHPAENGKSNFLNCY |
| | | VSGFHPSDIEVDLLKNGERIEKVEH |
| | | SDLSFSKDWSFYLLYYTEFTPTEKD |
| | | EYACRVNHVTLSQPKIVKWDRDMGG |
| | | GGSGGGGSGGGGSGSHSMRYFDTAV |
| | | SRPGRGEPRFISVGYVDDTQFVRFD |
| | | SDAASPRGEPRAPWVEQEGPEYWDR |
| | | ETQKYKRQAQADRVSLRNLRGAYNQ |
| | | SEDGSHTLQRMSGCDLGPDGRLLRG |
| | | YDQSAYDGKDYIALNEDLRSWTAAD |
| | | TAAQITQRKLEAARAAEQLRAYLEG |
| | | TCVEWLRRYLENGKETLQRAEPPKT |
| | | HVTHHPLSDHEATLRCWALGFYPAE |
| | | ITLTWQRDGEDQTQDTELVETRPAG |
| | | DGTFQKWAAVVVPSGQEQRYTCHMQ |
| | | HEGLQEPLTLSWEPSENLYFQGHHH |
| | | HHH |
| Synthetic | 34 | SYRPGTVALGGGGSGGGGSGGGGSI |
| HLA | | QRTPKIQVYSRHPAENGKSNFLNCY |
| | | VSGFHPSDIEVDLLKNGERIEKVEH |
| | | SDLSFSKDWSFYLLYYTEFTPTEKD |
| | | EYACRVNHVTLSQPKIVKWDRDMGG |
| | | GGSGGGGSGGGGSGSHSMRYFDTAV |
| | | SRPGRGEPRFISVGYVDDTQFVRFD |
| | | SDAASPRGEPRAPWVEQEGPEYWDR |
| | | ETQKYKRQAQADRVSLRNLRGAYNQ |
| | | SEDGSHTLQRMSGCDLGPDGRLLRG |
| | | YDQSAYDGKDYIALNEDLRSWTAAD |
| | | TAAQITQRKLEAARAAEQLRAYLEG |
| | | TCVEWLRRYLENGKETLQRAEPPKT |
| | | HVTHHPLSDHEATLRCWALGFYPAE |
| | | ITLTWQRDGEDQTAKTELVETRPAG |
| | | DGTFQKWAAVVVPSGQEQRYTCHMQ |
| | | HEGLQEPLTLSWEPSENLYFQGHHH |
| | | HHH |
| Synthetic | 35 | SYRPGTVALGCGGSGGGGSGGGGSI |
| HLA | | QRTPKIQVYSRHPAENGKSNFLNCY |
| | | VSGFHPSDIEVDLLKNGERIEKVEH |
| | | SDLSFSKDWSFYLLYYTEFTPTEKD |
| | | EYACRVNHVTLSQPKIVKWDRDMGG |
| | | GGSGGGGSGGGGSGSHSMRYFDTAV |
| | | SRPGRGEPRFISVGYVDDTQFVRFD |
| | | SDAASPRGEPRAPWVEQEGPEYWDR |
| | | ETQKYKRQAQADRVSLRNLRGCYNQ |
| | | SEDGSHTLQRMSGCDLGPDGRLLRG |
| | | YDQSAYDGKDYIALNEDLRSWTAAD |
| | | TAAQITQRKLEAARAAEQLRAYLEG |
| | | TCVEWLRRYLENGKETLQRAEPPKT |
| | | HVTHHPLSDHEATLRCWALGFYPAE |
| | | ITLTWQRDGEDQTAKTELVETRPAG |
| | | DGTFQKWAAVVVPSGQEQRYTCHMQ |
| | | HEGLQEPLTLSWEPSENLYFQGHHH |
| | | HHH |
| Synthetic | 36 | SYRPGTVALGGGGSGGGGSGSIQRT |
| HLA | | PKIQVYSRHPAENGKSNFLNCYVSG |
| | | FHPSDIEVDLLKNGERIEKVEHSDL |
| | | SFSKDWSFYLLYYTEFTPTEKDEYA |
| | | CRVNHVTLSQPKIVKWDRDMGGGGS |
| | | GGGGSGGGGSGSHSMRYFDTAVSRP |
| | | GRGEPRFISVGYVDDTQFVRFDSDA |
| | | ASPRGEPRAPWVEQEGPEYWDRETQ |
| | | KYKRQAQADRVSLRNLRGAYNQSED |
| | | GSHTLQRMSGCDLGPDGRLLRGYDQ |
| | | SAYDGKDYIALNEDLRSWTAADTAA |
| | | QITQRKLEAARAAEQLRAYLEGTCV |
| | | EWLRRYLENGKETLQRAEPPKTHVT |
| | | HHPLSDHEATLRCWALGFYPAEITL |
| | | TWQRDGEDQTQDTELVETRPAGDGT |
| | | FQKWAAVVVPSGQEQRYTCHMQHEG |
| | | LQEPLTLSWEPSENLYFQGHHHHHH |
| Synthetic | 37 | SYRPGTVALGGGGSGGGGSGSIQRT |
| HLA | | PKIQVYSRHPAENGKSNFLNCYVSG |
| | | FHPSDIEVDLLKNGERIEKVEHSDL |
| | | SFSKDWSFYLLYYTEFTPTEKDEYA |
| | | CRVNHVTLSQPKIVKWDRDMGGGGS |
| | | GGGGSGGGGSGSHSMRYFDTAVSRP |
| | | GRGEPRFISVGYVDDTQFVRFDSDA |
| | | ASPRGEPRAPWVEQEGPEYWDRETQ |
| | | KYKRQAQADRVSLRNLRGAYNQSED |
| | | GSHTLQRMSGCDLGPDGRLLRGYDQ |
| | | SAYDGKDYIALNEDLRSWTAADTAA |
| | | QITQRKLEAARAAEQLRAYLEGTCV |
| | | EWLRRYLENGKETLQRAEPPKTHVT |
| | | HHPLSDHEATLRCWALGFYPAEITL |
| | | TWQRDGEDQTAKTELVETRPAGDGT |
| | | FQKWAAVVVPSGQEQRYTCHMQHEG |
| | | LQEPLTLSWEPSENLYFQGHHHHHH |
| Synthetic | 38 | SYRPGTVALGGGGSGGGGSGSIQRT |
| HLA | | PKIQVYSRHPAENGKSNFLNCYVSG |
| | | FHPSDIEVDLLKNGERIEKVEHSDL |
| | | SFSKDWSFYLLYYTEFTPTEKDEYA |
| | | CRVNHVTLSQPKIVKWDRDMGGGGS |
| | | GGGGSGGGGSGSHSMRYFDTAVSRP |
| | | GRGEPRFISVGYVDDTQFVRFDSDA |
| | | ASPRGEPRAPWVEQEGPEYWDRETQ |
| | | KYKRQAQADRVSLRNLRGAYNQSED |
| | | GSHTLQRMSGCDLGPDGRLLRGYDQ |
| | | SAYDGKDYIALNEDLRSWTAADTAA |
| | | QITQRKLEAARAAEQLRAYLEGTCV |
| | | EWLRRYLENGKETLQRAEPPKTHVT |
| | | HHPLSDHEATLRCWALGFYPAEITL |
| | | TWQRDGEDQTAKTELVETRPAGDGT |
| | | FQKWAAVVVPSGQEQRYTCHMQHEG |
| | | LQEPLTLSWEPSENLYFQGHHHHHH |
| Synthetic | 39 | SYRPGTVALGGGGSGGGGSIQRTPK |
| HLA | | IQVYSRHPAENGKSNFLNCYVSGFH |
| | | PSDIEVDLLKNGERIEKVEHSDLSF |
| | | SKDWSFYLLYYTEFTPTEKDEYACR |
| | | VNHVTLSQPKIVKWDRDMGGGGSGG |
| | | GGSGGGGSGSHSMRYFDTAVSRPGR |
| | | GEPRFISVGYVDDTQFVRFDSDAAS |
| | | PRGEPRAPWVEQEGPEYWDRETQKY |
| | | KRQAQADRVSLRNLRGAYNQSEDGS |
| | | HTLQRMSGCDLGPDGRLLRGYDQSA |
| | | YDGKDYIALNEDLRSWTAADTAAQI |
| | | TQRKLEAARAAEQLRAYLEGTCVEW |
| | | LRRYLENGKETLQRAEPPKTHVTHH |
| | | PLSDHEATLRCWALGFYPAEITLTW |
| | | QRDGEDQTQDTELVETRPAGDGTFQ |
| | | KWAAVVVPSGQEQRYTCHMQHEGLQ |
| | | EPLTLSWEPSENLYFQGHHHHHH |
| Synthetic | 40 | SYRPGTVALGGGGSGGGGSIQRTPK |
| HLA | | IQVYSRHPAENGKSNFLNCYVSGFH |
| | | PSDIEVDLLKNGERIEKVEHSDLSF |
| | | SKDWSFYLLYYTEFTPTEKDEYACR |
| | | VNHVTLSQPKIVKWDRDMGGGGGGG |
| | | GSGGGGSGSHSMRYFDTAVSRPGRG |
| | | EPRFISVGYVDDTQFVRFDSDAASP |
| | | RGEPRAPWVEQEGPEYWDRETQKYK |
| | | RQAQADRVSLRNLRGAYNQSEDGSH |
| | | TLQRMSGCDLGPDGRLLRGYDQSAY |
| | | DGKDYIALNEDLRSWTAADTAAQIT |
| | | QRKLEAARAAEQLRAYLEGTCVEWL |
| | | RRYLENGKETLQRAEPPKTHVTHHP |
| | | LSDHEATLRCWALGFYPAEITLTWQ |
| | | RDGEDQTQDTELVETRPAGDGTFQK |
| | | WAAVVVPSGQEQRYTCHMQHEGLQE |
| | | PLTLSWEPSENLYFQGHHHHHH |
| Synthetic | 41 | SYRPGTVALGCGGSGGGGSIQRTPK |
| HLA | | IQVYSRHPAENGKSNFLNCYVSGFH |
| | | PSDIEVDLLKNGERIEKVEHSDLSF |
| | | SKDWSFYLLYYTEFTPTEKDEYACR |
| | | VNHVTLSQPKIVKWDRDMGGGGSGG |
| | | GGSGGGGSGSHSMRYFDTAVSRPGR |
| | | GEPRFISVGYVDDTQFVRFDSDAAS |
| | | PRGEPRAPWVEQEGPEYWDRETQKY |
| | | KRQAQADRVSLRNLRGCYNQSEDGS |
| | | HTLQRMSGCDLGPDGRLLRGYDQSA |
| | | YDGKDYIALNEDLRSWTAADTAAQI |
| | | TQRKLEAARAAEQLRAYLEGTCVEW |
| | | LRRYLENGKETLQRAEPPKTHVTHH |
| | | PLSDHEATLRCWALGFYPAEITLTW |
| | | QRDGEDQTAKTELVETRPAGDGTFQ |
| | | KWAAVVVPSGQEQRYTCHMQHEGLQ |
| | | EPLTLSWEPSENLYFQGHHHHHH |
| Synthetic | 42 | GSHSMRYFDTAVSRPGRGEPRFISV |
| HLA | and | GYVDDTQFVRFDSDAASPRGEPRAP |
| | 55 | WVEQEGPEYWDRETQKYKRQAQADR |
| | | VSLRNLRGAYNQSEDGSHTLQRMSG |
| | | CDLGPDGRLLRGYDQSAYDGKDYIA |
| | | LNEDLRSWTAADTAAQITQRKLEAA |
| | | RAAEQLRAYLEGTCVEWLRRYLENG |
| | | KETLQRAEPPKTHVTHHPLSDHEAT |
| | | LRCWALGFYPAEITLTWQRDGEDQT |
| | | QDTELVETRPAGDGTFQKWAAVVVP |
| | | SGQEQRYTCHMQHEGLQEPLTLSWE |
| | | PSENLYFQGHHHHHH-stop-GLND |
| | | IFEAQKIEWHE |
| Synthetic | 43 | GSHSMRYFDTAVSRPGRGEPRFISV |
| HLA | and | GYVDDTQFVRFDSDAASPRGEPRAP |
| | 56 | WVEQEGPEYWDRETQKYKRQAQADR |
| | | VSLRNLRGAYNQSEDGSHTLQRMSG |
| | | CDLGPDGRLLRGYDQSAYDGKDYIA |
| | | LNEDLRSWTAADTAAQITQRKLEAA |
| | | RAAEQLRAYLEGTCVEWLRRYLENG |
| | | KETLQRAEPPKTHVTHHPLSDHEAT |
| | | LRCWALGFYPAEITLTWQRDGEDQT |
| | | AKTELVETRPAGDGTFQKWAAVVVP |
| | | SGQEQRYTCHMQHEGLQEPLTLSWE |
| | | PSENLYFQGHHHHHH-stop-GLND |
| | | IFEAQKIEWHE |
| Synthetic | 44 | GSHSMRYFDTAVSRPGRGEPRFISV |
| HLA | and | GYVDDTQFVRFDSDAASPRGEPRAP |
| | 57 | WVEQEGPEYWDRETQKYKRQAQADR |
| | | VSLRNLRGCYNQSEDGSHTLQRMSG |
| | | CDLGPDGRLLRGYDQSAYDGKDYIA |
| | | LNEDLRSWTAADTAAQITQRKLEAA |
| | | RAAEQLRAYLEGTCVEWLRRYLENG |
| | | KETLQRAEPPKTHVTHHPLSDHEAT |
| | | LRCWALGFYPAEITLTWQRDGEDQT |
| | | AKTELVETRPAGDGTFQKWAAVVVP |
| | | SGQEQRYTCHMQHEGLQEPLTLSWE |
| | | PSENLYFQGHHHHHH-stop-GLND |
| | | IFEAQKIEWHE |

### Peptides

Provided herein, in one aspect, are peptides configured to weaken or inhibit HLA activity.

In some embodiments, the peptide does not elicit a T-cell response when the peptide is interrogated by one or more T-cells. In some embodiments, the peptide is incapable of activating the one or more T-cells. In some embodiments, the peptide is capable of binding to a receptor of the one or more T-cells, and wherein the binding is insufficient to activate the one or more T-cells.

In some embodiments, a peptide is configured to covalently bind to HLA. In some embodiments, the peptide is configured to bind to the N-terminus of the beta chain of HLA Class II or the N-terminus of beta-2M chain of HLA Class 1. In some embodiments, the peptide is configured to be specific for the MHC binding groove but does not comprise the correct TCR-facing/solvent exposed amino acids required for recognition by the T-cell Receptor (TCR). The presence of specific “anchor residues” in the MHC binding groove of HLA act to anchor the bound peptide.

In some embodiments, residues in the peptide can be altered in order to configure the peptide so that a TCR does not recognize and/or bind to the peptide-bound MHC (pMHC). Although the TCR interacts with residues in the MHC, 1-3 TCR-facing, solvent-exposed residues from the peptide also contribute directly to the TCR interaction. In some embodiments, the TCR-facing residues of the peptide are configured to antagonize TCR interaction.

In some embodiments, the peptide binds to one or more HLA binding groove domain residues of the human HLA class 1 heavy chain sequence. In some embodiments, the peptide modulates a conformation of the human HLA class 1 heavy chain sequence. In some embodiments, the conformation prevents the one or more T-cells from binding to the human HLA class 1 heavy chain sequence.

In some embodiments, the sequence of the bound peptide can affect the intrinsic flexibility of the pMHC. In some embodiments, the conformational flexibility of the pMHC facilitates TCR interaction. In some embodiments, the peptide configured to bind to HLA is further configured to increase the conformational variability of pMHC and to prevent TCR engagement.

In some embodiments, the peptide is about 8 amino acids in length to about 15 amino acids in length. In some embodiments, the peptide is about 8 amino acids in length to about 9 amino acids in length, about 8 amino acids in length to about 10 amino acids in length, about 8 amino acids in length to about 11 amino acids in length, about 8 amino acids in length to about 12 amino acids in length, about 8 amino acids in length to about 13 amino acids in length, about 8 amino acids in length to about 14 amino acids in length, about 8 amino acids in length to about 15 amino acids in length, about 9 amino acids in length to about 10 amino acids in length, about 9 amino acids in length to about 11 amino acids in length, about 9 amino acids in length to about 12 amino acids in length, about 9 amino acids in length to about 13 amino acids in length, about 9 amino acids in length to about 14 amino acids in length, about 9 amino acids in length to about 15 amino acids in length, about 10 amino acids in length to about 11 amino acids in length, about 10 amino acids in length to about 12 amino acids in length, about 10 amino acids in length to about 13 amino acids in length, about 10 amino acids in length to about 14 amino acids in length, about 10 amino acids in length to about 15 amino acids in length, about 11 amino acids in length to about 12 amino acids in length, about 11 amino acids in length to about 13 amino acids in length, about 11 amino acids in length to about 14 amino acids in length, about 11 amino acids in length to about 15 amino acids in length, about 12 amino acids in length to about 13 amino acids in length, about 12 amino acids in length to about 14 amino acids in length, about 12 amino acids in length to about 15 amino acids in length, about 13 amino acids in length to about 14 amino acids in length, about 13 amino acids in length to about 15 amino acids in length, or about 14 amino acids in length to about 15 amino acids in length. In some embodiments, the peptide is about 8 amino acids in length, about 9 amino acids in length, about 10 amino acids in length, about 11 amino acids in length, about 12 amino acids in length, about 13 amino acids in length, about 14 amino acids in length, or about 15 amino acids in length. In some embodiments, the peptide is at least about 8 amino acids in length, about 9 amino acids in length, about 10 amino acids in length, about 11 amino acids in length, about 12 amino acids in length, about 13 amino acids in length, or about 14 amino acids in length. In some embodiments, the peptide is at most about 9 amino acids in length, about 10 amino acids in length, about 11 amino acids in length, about 12 amino acids in length, about 13 amino acids in length, about 14 amino acids in length, or about 15 amino acids in length. In some embodiments, the peptide comprises greater than 14 amino acids.

In some embodiments, the use of unusually long peptides to bind to the MHC binding groove of HLA inhibits HLA activity. MHC-I typically binds peptides 8-10 amino acids in length, but can also bind non-canonical, longer peptides (e.g. 13 amino acids). The ends of such a long peptide bind to the MHC binding groove at the anchor residues, creating a “bulge” at the center of the peptide binding site. In such pMHC-TCR complexes, the TCR makes relatively few contacts with the MHC (typically with canonical, short peptides in such complexes the MHC heavy chain dominates the interface with TCR) and instead the interaction with TCR is dominated by the peptide directly. The bulged peptide also represents a steric challenge for TCR engagement. Given the dominance of peptide-TCR interactions in such a system, by selecting the peptide sequence at the bulge, it is possible to prevent TCR-binding. In some embodiments, the peptide is configured to block and/or silence the amino acids of HLA required for molecular contacts with TCR and/or the peptide does not comprise amino acid residues sufficient for TCR binding and/or activity. In some embodiments, the peptide is configured to increase the conformational heterogeneity of HLA in this region as to render the HLA incapable of TCR binding and/or activity. In some embodiments, the peptide is configured to do any combination of the functions described above.

In some embodiments, the peptide is coupled to the complex by a disulfide bond.

In some embodiments, the complex further comprises a regulatory peptide. In some embodiments, the regulatory peptide is an apoptosis-inducing peptide. In some embodiments, the apoptosis-inducing peptide acts as a “kill switch” for the complex.

In some embodiments, the peptide comprises a sequence at least about 70%, about 80%, about 85%, about 86%, about 87%, about 88%, about 89%, about 90%, about 91%, about 92%, about 93%, about 94%, about 95%, about 96%, about 97%, about 98%, about 99%, about 99.9%, or about 100% identical to a sequence listed in Table B below.

TABLE B
Peptides to weaken or inhibit HLA activity

| | SEQ ID | |
|---|---|---|
| Description | NO. | Sequence |
| influenza A virus M1 | 45 | GILGFVFTL |
| (58-66) | | |
| peptide | | |
| histone H3 peptide | 46 | RYRPGTVAL |
| histone H3 peptide | 47 | SYRPGTVAL |
| (modified) | | |

### Linkers

In some embodiments, the complex comprises one or more linkers between the peptide and the human HLA class 1 heavy chain sequence. In some embodiments, the one or more linkers are configured to resist proteolytic cleavage. In some embodiments, the one or more linkers comprise a conformation configured to not block one or more killer-cell immunoglobulin-like receptor (KIR) binding sites on the human HLA class 1 heavy chain sequence. In some embodiments, the one or more linkers are structurally stable. In some embodiments, the one or more linkers are rigid. In some embodiments, the one or more linkers possess limited flexibility. In some embodiments, the structural stability, rigidity, and limited flexibility of the one or more linkers increase resistance to proteolytic degradation.

In some embodiments, a linker of the one or more linkers is displaced between the peptide and the human β2-microglobulin sequence, between the human β2-microglobulin sequence and the human HLA class 1 heavy chain sequence, or both. In some embodiments, a linker of the one or more linkers is displaced between the peptide and the human β2-microglobulin sequence. In some embodiments, a linker of the one or more linkers is displaced between the human β2-microglobulin sequence and the human HLA class 1 heavy chain sequence. In some embodiments, a first linker of the one or more linkers is displaced between the peptide and the human β2-microglobulin sequence and a second linker of the one or more linkers is displaced between the human β2-microglobulin sequence and the human HLA class 1 heavy chain sequence. In some embodiments, the second linker comprises a conformation configured to resist proteolytic cleavage. In some embodiments, the second linker is further configured to not block one or more killer-cell immunoglobulin-like receptor (KIR) binding sites on the human HLA class 1 heavy chain sequence. In some embodiments, the conformation of the second linker allows for MR binding to the human HLA class 1 heavy chain sequence, preventing a “missing self” immune response. In some embodiments, the conformation of the second linker prevents attack by one or more natural killer cells.

In some embodiments, a linker of the one or more linkers comprises a sequence at least about 70%, about 80%, about 85%, about 86%, about 87%, about 88%, about 89%, about 90%, about 91%, about 92%, about 93%, about 94%, about 95%, about 96%, about 97%, about 98%, about 99%, about 99.9%, or about 100% identical to a sequence listed in Table C below.

TABLE C
Linker sequences

| Description | SEQ ID NO. | Sequence |
|---|---|---|
| Linker | 48 | GGGGSGGGGSGGGGS |
| Linker | 49 | GGGGSGGGGSGGGGSGGGGS |
| Linker | 50 | GGGGSGGGGS |
| Linker | 51 | GCGGSGGGGS |
| Linker | 52 | GGGGSGGGGSGS |
| Linker | 53 | GCGGSGGGGSGS |
| Linker | 54 | GCGGSGGGGSGGGGS |

In some embodiments, the one or more human leukocyte antigens (HLAs) comprise one or more mutations, wherein the one or more mutations inhibit the one or more HLAs from eliciting a T-cell response when the complex is interrogated by one or more CD8 cells. In some embodiments, the one or more mutations comprises a mutation of one or more of amino acid residues 115, 122, 128, 194, 197, 198, 212, 214, 222, 223, 224, 225, 226, 227, 228, 229, 230, 231, 232, 233, 243, 245, 248, 262, or any combination thereof. In some embodiments, the one or more mutations comprises a mutation of amino acid residue 115. In some embodiments, the one or more mutations comprises a mutation of amino acid residue 122. In some embodiments, the one or more mutations comprises a mutation of amino acid residue 128. In some embodiments, the one or more mutations comprises a mutation of amino acid residue 194. In some embodiments, the one or more mutations comprises a mutation of amino acid residue 197. In some embodiments, the one or more mutations comprises a mutation of amino acid residue 198. In some embodiments, the one or more mutations comprises a mutation of amino acid residue 212. In some embodiments, the one or more mutations comprises a mutation of amino acid residue 214. In some embodiments, the one or more mutations comprises a mutation of amino acid residue 222. In some embodiments, the one or more mutations comprises a mutation of amino acid residue 223. In some embodiments, the one or more mutations comprises a mutation of amino acid residue 224. In some embodiments, the one or more mutations comprises a mutation of amino acid residue 225. In some embodiments, the one or more mutations comprises a mutation of amino acid residue 226. In some embodiments, the one or more mutations comprises a mutation of amino acid residue 227. In some embodiments, the one or more mutations comprises a mutation of amino acid residue 228. In some embodiments, the one or more mutations comprises a mutation of amino acid residue 229. In some embodiments, the one or more mutations comprises a mutation of amino acid residue 230. In some embodiments, the one or more mutations comprises a mutation of amino acid residue 231. In some embodiments, the one or more mutations comprises a mutation of amino acid residue 232. In some embodiments, the one or more mutations comprises a mutation of amino acid residue 233. In some embodiments, the one or more mutations comprises a mutation of amino acid residue 243. In some embodiments, the one or more mutations comprises a mutation of amino acid residue 245. In some embodiments, the one or more mutations comprises a mutation of amino acid residue 248. In some embodiments, the one or more mutations comprises a mutation of amino acid residue 262.

In some embodiments, the complex further comprises one or more proteins or fragments thereof that inhibit an immune response by the complement system. In some embodiments, the one or more proteins or fragments thereof are selected from CD48, CD59, or a combination thereof. In some embodiments, the one or more proteins or fragments thereof is CD48. In some embodiments, the one or more proteins or fragments thereof is CD59. In some embodiments, the one or more proteins or fragments thereof are CD48 and CD59.

In some embodiments, the peptide comprises a second amino acid residue selected from L, M, S, I, F, T, V, and Y. In some embodiments, the second amino acid residue is selected from T, V, and Y. In some embodiments, the peptide comprises a last amino acid residue selected from V, I, F, W, Y, L, R, and K. In some embodiments, the last amino acid residue is selected from Y, L, R, and K.

In some embodiments, the peptide comprises a second amino acid residue selected from E, P, L, Q, A, R, H, S, T, V, M, D, and K. In some embodiments, the second amino acid residue is selected from E, P, L, Q, A, R, and H. In some embodiments, the peptide comprises a last amino acid residue selected from V, L, F, A, I, Y, M, W, P, and R. In some embodiments, the last amino acid residue is selected from V, L, and F.

In some embodiments, the peptide comprises a second amino acid residue selected from A, Y, S, T, V, I, L, F, Q, R, N, and W. In some embodiments, the second amino acid residue is selected from A and Y. In some embodiments, the peptide comprises a last amino acid residue selected from L, V, M, F, Y, and I. In some embodiments, the last amino acid residue is L.

### Nucleic Acid Molecules

Provided herein, in another aspect, is a nucleic acid molecule encoding the complex provided herein.

In some embodiments, the nucleic acid molecule comprises a deletion in the endogenous HLA locus. In some embodiments, the deletion comprises a deletion in the endogenous HLA-A, HLA-B, or HLA-C locus, or any combination thereof. In some embodiments, the deletion comprises a deletion in the endogenous HLA-A locus. In some embodiments, the deletion comprises a deletion in the endogenous HLA-B locus. In some embodiments, the deletion comprises a deletion in the endogenous HLA-C locus. In some embodiments, the deletion comprises a deletion in the endogenous HLA-A locus and the HLA-B locus. In some embodiments, the deletion comprises a deletion in the endogenous HLA-A locus and the HLA-C locus. In some embodiments, the deletion comprises a deletion in the endogenous HLA-B locus and the HLA-C locus. In some embodiments, the deletion comprises a deletion in the endogenous HLA-A locus, the HLA-B locus, and the HLA-C locus. In some embodiments, the deletion is complete deletion of the endogenous HLA locus.

In some embodiments, the nucleic acid molecule further comprises a sequence encoding a human HLA class 1 heavy chain sequence. In some embodiments, the sequence encoding a human HLA class 1 heavy chain sequence comprises an HLA-A sequence, an HLA-B sequence, an HLA-C sequence, or any combination thereof. In some embodiments, the sequence encoding a human HLA class 1 heavy chain sequence comprises an HLA-A sequence. In some embodiments, the sequence encoding a human HLA class 1 heavy chain sequence comprises an HLA-B sequence. In some embodiments, the sequence encoding a human HLA class 1 heavy chain sequence comprises an HLA-C sequence. In some embodiments, the sequence encoding a human HLA class 1 heavy chain sequence comprises an HLA-A sequence and an HLA-B sequence. In some embodiments, the sequence encoding a human HLA class 1 heavy chain sequence comprises an HLA-A sequence and an HLA-C sequence. In some embodiments, the sequence encoding a human HLA class 1 heavy chain sequence comprises an HLA-B sequence and an HLA-C sequence. In some embodiments, the sequence encoding a human HLA class 1 heavy chain sequence comprises an HLA-A sequence, an HLA-B sequence, and an HLA-C sequence.

In some embodiments, the sequence encoding a human HLA class 1 heavy chain sequence comprises multiple alleles of an HLA-A sequence, an HLA-B sequence, an HLA-C sequence, or any combination thereof. In some embodiments, the sequence encoding a human HLA class 1 heavy chain sequence comprises multiple alleles of an HLA-A sequence. In some embodiments, the sequence encoding a human HLA class 1 heavy chain sequence comprises multiple alleles of an HLA-B sequence. In some embodiments, the sequence encoding a human HLA class 1 heavy chain sequence comprises multiple alleles of an HLA-C sequence. In some embodiments, the sequence encoding a human HLA class 1 heavy chain sequence comprises multiple alleles of an HLA-A sequence and multiple alleles of an HLA-B sequence. In some embodiments, the sequence encoding a human HLA class 1 heavy chain sequence comprises multiple alleles of an HLA-A sequence and multiple alleles of an HLA-C sequence. In some embodiments, the sequence encoding a human HLA class 1 heavy chain sequence comprises multiple alleles of an HLA-B sequence and multiple alleles of an HLA-C sequence. In some embodiments, the sequence encoding a human HLA class 1 heavy chain sequence comprises multiple alleles of an HLA-A sequence, multiple alleles of an HLA-B sequence, and multiple alleles of an HLA-C sequence.

In some embodiments, the alleles of an HLA-A sequence are selected from HLA-A*02:01, HLA-A*01:01, HLA-A*03:01, HLA-A*11:01, HLA-A*24:02, HLA-A*29:02, HLA-A*26:01, HLA-A*32:01, HLA-A*23:01, HLA-A*68:02, HLA-A*30:01, HLA-A*30:02, HLA-A*34:02, HLA-A*31:01, HLA-A*33:03, HLA-A*02:07, HLA-A*02:06, and HLA-A*02:03.

In some embodiments, the alleles of an HLA-B sequence are selected from HLA-B*44:02, HLA-B*07:02, HLA-B*08:01, HLA-B*40:01, HLA-B*35:01, HLA-B*51:01, HLA-B*15:01, HLA-B*53:01, HLA-B*15:03, HLA-B*58:01, HLA-B*45:01, HLA-B*42:01, HLA-B*44:03, HLA-B*18:01, HLA-B*52:01, HLA-B*14:02, HLA-B*46:01, HLA-B*38:02, and HLA-B*15:02.

In some embodiments, the alleles of an HLA-C sequence are selected from HLA-C*07:01, HLA-C*07:02, HLA-C*04:01, HLA-C*05:01, HLA-C*03:04, HLA-C*06:02, HLA-C*03:03, HLA-C*12:03, HLA-C*08:02, HLA-C*02:02, HLA-C*16:01, HLA-C*17:01, HLA-C*01:02, HLA-C*02:01, HLA-C*08:01, HLA-C*03:02, and HLA-C*14:02.

In some embodiments, the sequence encoding a human HLA class 1 heavy chain sequence comprises an HLA-A sequence, wherein the HLA-A sequence is displaced between the HLA-B sequence and the HLA-C sequence.

In some embodiments, the sequence encoding a human HLA class 1 heavy chain sequence comprises fewer than 1700 base pairs (bp). In some embodiments, the sequence encoding a human HLA class 1 heavy chain sequence comprises fewer than 1600 base pairs (bp). In some embodiments, the sequence encoding a human HLA class 1 heavy chain sequence comprises fewer than 1500 base pairs (bp). In some embodiments, the sequence encoding a human HLA class 1 heavy chain sequence comprises fewer than 1400 base pairs (bp). In some embodiments, the sequence encoding a human HLA class 1 heavy chain sequence comprises fewer than 1300 base pairs (bp). In some embodiments, the sequence encoding a human HLA class 1 heavy chain sequence comprises fewer than 1200 base pairs (bp). In some embodiments, the sequence encoding a human HLA class 1 heavy chain sequence comprises fewer than 1100 base pairs (bp). In some embodiments, the sequence encoding a human HLA class 1 heavy chain sequence comprises fewer than 1000 base pairs (bp). In some embodiments, the sequence encoding a human HLA class 1 heavy chain sequence comprises fewer than 900 base pairs (bp). In some embodiments, the sequence encoding a human HLA class 1 heavy chain sequence comprises fewer than 800 base pairs (bp). In some embodiments, the sequence encoding a human HLA class 1 heavy chain sequence comprises fewer than 700 base pairs (bp). In some embodiments, the sequence encoding a human HLA class 1 heavy chain sequence comprises fewer than 600 base pairs (bp). In some embodiments, the sequence encoding a human HLA class 1 heavy chain sequence comprises fewer than 500 bp. In some embodiments, the sequence encoding a human HLA class 1 heavy chain sequence comprises fewer than 450 bp. In some embodiments, the sequence encoding a human HLA class 1 heavy chain sequence comprises fewer than 400 bp. In some embodiments, the sequence encoding a human HLA class 1 heavy chain sequence comprises fewer than 350 bp. In some embodiments, the sequence encoding a human HLA class 1 heavy chain sequence comprises fewer than 300 bp. In some embodiments, the sequence encoding a human HLA class 1 heavy chain sequence comprises fewer than 250 bp. In some embodiments, the sequence encoding a human HLA class 1 heavy chain sequence comprises fewer than 200 bp. In some embodiments, the sequence encoding a human HLA class 1 heavy chain sequence comprises fewer than 150 bp. In some embodiments, the sequence encoding a human HLA class 1 heavy chain sequence comprises fewer than 100 bp. In some embodiments, the sequence encoding a human HLA class 1 heavy chain sequence comprises fewer than 50 bp.

In some embodiments, the HLA-A sequence, HLA-B sequence, HLA-C sequences, or combination thereof comprises one or more flanking sequences. In some embodiments, the HLA-A sequence comprises one or more flanking sequences. In some embodiments, the HLA-B sequence comprises one or more flanking sequences. In some embodiments, the HLA-C sequence comprises one or more flanking sequences. In some embodiments, the HLA-A sequence and the HLA-B sequence comprise one or more flanking sequences. In some embodiments, the HLA-A sequence and the HLA-C sequence comprise one or more flanking sequences. In some embodiments, the HLA-B sequence and the HLA-C sequence comprise one or more flanking sequences. In some embodiments, the HLA-A sequence, the HLA-B sequence, and the HLA-C comprise one or more flanking sequences.

In some embodiments, the one or more flanking sequences comprise an endogenous HLA sequence. In some embodiments, the one or more flanking sequences are specific to one or more promoters. In some embodiments, the promoters comprise an HLA-A promoter, HLA-B promoter, HLA-C promoter, or combination thereof. In some embodiments, the HLA-A sequence comprises an endogenous HLA-A promoter. In some embodiments, the HLA-B sequence comprises an endogenous HLA-B promoter. In some embodiments, the HLA-C sequence comprises an endogenous HLA-C promoter. In some embodiments, the HLA-A sequence comprises an endogenous HLA-A promoter and the HLA-B sequence comprises an endogenous HLA-B promoter. In some embodiments, the HLA-A sequence comprises an endogenous HLA-A promoter and the HLA-C sequence comprises an endogenous HLA-C promoter. In some embodiments, the HLA-B sequence comprises an endogenous HLA-B promoter and the HLA-C sequence comprises an endogenous HLA-C promoter. In some embodiments, the HLA-A sequence comprises an endogenous HLA-A promoter, the HLA-B sequence comprises an endogenous HLA-B promoter, and the HLA-C sequence comprises an endogenous HLA-C promoter.

In some embodiments, the sequence encoding a human HLA class 1 heavy chain sequence does not comprise at least a portion of the HLA-A sequence, HLA-B sequence, HLA-C sequence, or combination thereof. In some embodiments, the sequence encoding a human HLA class 1 heavy chain sequence does not comprise at least a portion of the HLA-A sequence. In some embodiments, the sequence encoding a human HLA class 1 heavy chain sequence does not comprise at least a portion of the HLA-B sequence. In some embodiments, the sequence encoding a human HLA class 1 heavy chain sequence does not comprise at least a portion of the HLA-C sequence. In some embodiments, the sequence encoding a human HLA class 1 heavy chain sequence does not comprise at least a portion of the HLA-A sequence or the HLA-B sequence. In some embodiments, the sequence encoding a human HLA class 1 heavy chain sequence does not comprise at least a portion of the HLA-A sequence or the HLA-C sequence. In some embodiments, the sequence encoding a human HLA class 1 heavy chain sequence does not comprise at least a portion of the HLA-B sequence or the HLA-C sequence. In some embodiments, the sequence encoding a human HLA class 1 heavy chain sequence does not comprise at least a portion of the HLA-A sequence, the HLA-B sequence, or the HLA-C sequence.

In some embodiments, the nucleic acid molecule further comprises a sequence encoding a human β2-microglobulin peptide. In some embodiments, the nucleic acid molecule further comprises a sequence encoding an endogenous human β2-microglobulin peptide.

In some embodiments, the nucleic acid molecule further comprises a sequence encoding a peptide.

In some embodiments, the nucleic acid molecule further comprises one or more sequences encoding one or more linkers between the sequence encoding the peptide and the sequence encoding the human HLA class 1 heavy chain sequence. In some embodiments, a sequence of the one or more sequences encoding one or more linkers is displaced between the sequence encoding the peptide and the sequence encoding the human β2-microglobulin peptide, between the sequence encoding the human β2-microglobulin peptide and the sequence encoding the human HLA class 1 heavy chain sequence, or both. In some embodiments, a sequence of the one or more sequences encoding one or more linkers is displaced between the sequence encoding the peptide and the sequence encoding the human β2-microglobulin peptide. In some embodiments, a sequence of the one or more sequences encoding one or more linkers is displaced between the sequence encoding the human β2-microglobulin peptide and the sequence encoding the human HLA class 1 heavy chain sequence. In some embodiments, a first sequence of the one or more sequences encoding one or more linkers is displaced between the sequence encoding the peptide and the sequence encoding the human β2-microglobulin peptide, and a second sequence of the one or more sequences encoding one or more linkers is displaced between the sequence encoding the human β2-microglobulin peptide and the sequence encoding the human HLA class 1 heavy chain sequence.

In some embodiments, the nucleic acid molecule further comprises a sequence encoding one or more immune checkpoint agonists. In some embodiments, the nucleic acid molecule further comprises a sequence encoding CD47. In some embodiments, the nucleic acid molecule further comprises a sequence encoding PD-L1. In some embodiments, the nucleic acid molecule further comprises a sequence encoding A2AR. In some embodiments, the nucleic acid molecule further comprises a sequence encoding B7-H3. In some embodiments, the nucleic acid molecule further comprises a sequence encoding B7-H4. In some embodiments, the nucleic acid molecule further comprises a sequence encoding BTLA. In some embodiments, the nucleic acid molecule further comprises a sequence encoding CTLA-4. In some embodiments, the nucleic acid molecule further comprises a sequence encoding IDO. In some embodiments, the nucleic acid molecule further comprises a sequence encoding MR. In some embodiments, the nucleic acid molecule further comprises a sequence encoding LAG3. In some embodiments, the nucleic acid molecule further comprises a sequence encoding NOX2. In some embodiments, the nucleic acid molecule further comprises a sequence encoding PD-1. In some embodiments, the nucleic acid molecule further comprises a sequence encoding TIM-3. In some embodiments, the nucleic acid molecule further comprises a sequence encoding VISTA. In some embodiments, the nucleic acid molecule further comprises a sequence encoding SIGLEC7.

In some embodiments, the nucleic acid molecule further comprises a sequence encoding one or more knocked out proteins corresponding to a receptor of the one or more immune checkpoint agonists. In some embodiments, the nucleic acid molecule further comprises a sequence encoding a knocked out CD47 receptor. In some embodiments, the nucleic acid molecule further comprises a sequence encoding a knocked out PD-L1 receptor. In some embodiments, the nucleic acid molecule further comprises a sequence encoding a knocked out A2AR receptor. In some embodiments, the nucleic acid molecule further comprises a sequence encoding a knocked out B7-H3 receptor. In some embodiments, the nucleic acid molecule further comprises a sequence encoding a knocked out B7-H4 receptor. In some embodiments, the nucleic acid molecule further comprises a sequence encoding a knocked out BTLA receptor. In some embodiments, the nucleic acid molecule further comprises a sequence encoding a knocked out CTLA-4 receptor. In some embodiments, the nucleic acid molecule further comprises a sequence encoding a knocked out IDO receptor. In some embodiments, the nucleic acid molecule further comprises a sequence encoding a knocked out MR receptor. In some embodiments, the nucleic acid molecule further comprises a sequence encoding a knocked out LAG3 receptor. In some embodiments, the nucleic acid molecule further comprises a sequence encoding a knocked out NOX2 receptor. In some embodiments, the nucleic acid molecule further comprises a sequence encoding a knocked out PD-1 receptor. In some embodiments, the nucleic acid molecule further comprises a sequence encoding a knocked out TIM-3 receptor. In some embodiments, the nucleic acid molecule further comprises a sequence encoding a knocked out VISTA receptor. In some embodiments, the nucleic acid molecule further comprises a sequence encoding a knocked out SIGLEC7 receptor.

In some embodiments, the sequence encoding the human HLA class 1 heavy chain sequence comprises an HLA-E sequence or a fragment thereof, an HLA-F sequence or a fragment thereof, an HLA-G sequence or a fragment thereof, or any combination thereof. In some embodiments, the sequence encoding the human HLA class 1 heavy chain sequence comprises an HLA-E sequence or a fragment thereof. In some embodiments, the sequence encoding the human HLA class 1 heavy chain sequence comprises an HLA-F sequence or a fragment thereof. In some embodiments, the sequence encoding the human HLA class 1 heavy chain sequence comprises an HLA-G sequence or a fragment thereof. In some embodiments, the sequence encoding the human HLA class 1 heavy chain sequence comprises an HLA-E sequence or a fragment thereof and an HLA-F sequence or a fragment thereof. In some embodiments, the sequence encoding the human HLA class 1 heavy chain sequence comprises an HLA-E sequence or a fragment thereof and an HLA-G sequence or a fragment thereof. In some embodiments, the sequence encoding the human HLA class 1 heavy chain sequence comprises an HLA-F sequence or a fragment thereof and an HLA-G sequence or a fragment thereof. In some embodiments, the sequence encoding the human HLA class 1 heavy chain sequence comprises an HLA-E sequence or a fragment thereof, an HLA-F sequence or a fragment thereof, and an HLA-G sequence or a fragment thereof.

In some embodiments, at least one of the HLA-E sequence or the fragment thereof, HLA-F sequence or the fragment thereof, HLA-G sequence or the fragment thereof, or any combination thereof is inhibited from eliciting a T-cell response when the complex is interrogated by one or more T-cells. In some embodiments, the HLA-E sequence or the fragment thereof is inhibited from inhibited from eliciting a T-cell response when the complex is interrogated by one or more T-cells. In some embodiments, the HLA-F sequence or the fragment thereof is inhibited from eliciting a T-cell response when the complex is interrogated by one or more T-cells. In some embodiments, the HLA-G sequence or the fragment thereof is inhibited from eliciting a T-cell response when the complex is interrogated by one or more T-cells. In some embodiments, the HLA-E sequence or the fragment thereof and the HLA-F sequence or the fragment thereof are inhibited from eliciting a T-cell response when the complex is interrogated by one or more T-cells. In some embodiments, the HLA-E sequence or the fragment thereof and the HLA-G sequence or the fragment thereof are inhibited from eliciting a T-cell response when the complex is interrogated by one or more T-cells. In some embodiments, the HLA-F sequence or the fragment thereof and the HLA-G sequence or the fragment thereof are inhibited from eliciting a T-cell response when the complex is interrogated by one or more T-cells. In some embodiments, the HLA-E sequence or the fragment thereof, the HLA-F sequence or the fragment thereof, and the HLA-G sequence or the fragment thereof are inhibited from eliciting a T-cell response when the complex is interrogated by one or more T-cells.

In some embodiments, the nucleic acid molecule further comprises a sequence encoding one or more knocked out proteins corresponding class II, major histocompatibility complex, transactivator (CIITA). In some embodiments, the entire class II, major histocompatibility complex, transactivator (CIITA) locus is knocked out.

In some embodiments, the nucleic acid molecule further comprises a sequence encoding a regulatory peptide. In some embodiments, the nucleic acid molecule further comprises a sequence encoding an apoptosis-inducing peptide. In some embodiments, the nucleic acid molecule further comprises a sequence encoding an apoptosis-inducing peptide to act as a “kill switch.”

In some embodiments, the nucleic acid molecule further comprises a sequence encoding an epitope configured to allow for detection of the complex. In some embodiments, the nucleic acid molecule further comprises a sequence encoding an epitope comprising 3,5-dinitrosalicylic acid.

In some embodiments, the nucleic acid molecule further comprises a sequence encoding one or more knocked out proteins. In some embodiments, the one or more knocked out proteins are selected from blood group A antigen and blood group B antigen. In some embodiments, the nucleic acid molecule further comprises a sequence encoding knocked out blood group A antigen. In some embodiments, the nucleic acid molecule further comprises a sequence encoding knocked out blood group B antigen.

In some embodiments, the nucleic acid molecule comprises,
a. the sequence encoding the peptide;
b. a first sequence encoding a first linker of the one or more sequences encoding one or more linkers;
c. the sequence encoding the human β2-microglobulin peptide;
d. a second sequence encoding a second linker of the one or more sequences encoding one or more linkers; and
e. the sequence encoding the human HLA class 1 heavy chain sequence.

Provided herein, in another aspect, is a nucleic acid molecule comprising a sequence encoding a complex comprising one or more Class 1 human leukocyte antigen (HLA) proteins, wherein the one or more Class 1 HLA proteins are inhibited from eliciting a T-cell response when the complex is interrogated by one or more T-cells, and wherein the nucleic acid molecule comprises,
a. a sequence encoding a peptide, wherein the peptide is incapable of activating the one or more T-cells;
b. a first sequence encoding a first linker; and
c. a sequence encoding one or more Class 1 HLA proteins;
   wherein the first linker comprises a conformation configured to not block one or more killer-cell immunoglobulin-like receptor (KIR) binding sites on the human HLA class 1 heavy chain sequence, and wherein the conformation is further configured to resist proteolytic cleavage.

In some embodiments, the nucleic acid molecule further comprises a sequence encoding a human β2-microglobulin peptide between the sequence encoding the linker and the sequence encoding the human HLA class 1 heavy chain sequence. In some embodiments, the nucleic acid molecule further comprises a sequence encoding an endogenous human β2-microglobulin peptide between the sequence encoding the linker and the sequence encoding the human HLA class 1 heavy chain sequence.

In some embodiments, the sequence encoding a human HLA class 1 heavy chain sequence comprises one or more mutations, wherein the one or more mutations inhibit the human HLA class 1 heavy chain sequence from eliciting a T-cell response when the human HLA class 1 heavy chain sequence is interrogated by one or more CD8 cells. In some embodiments, the one or more mutations comprises a mutation of one or more of amino acid residues 115, 122, 128, 194, 197, 198, 212, 214, 222, 223, 224, 225, 226, 227, 228, 229, 230, 231, 232, 233, 243, 245, 248, 262, or any combination thereof. In some embodiments, the one or more mutations comprises a mutation of amino acid residue 115. In some embodiments, the one or more mutations comprises a mutation of amino acid residue 122. In some embodiments, the one or more mutations comprises a mutation of amino acid residue 128. In some embodiments, the one or more mutations comprises a mutation of amino acid residue 194. In some embodiments, the one or more mutations comprises a mutation of amino acid residue 197. In some embodiments, the one or more mutations comprises a mutation of amino acid residue 198. In some embodiments, the one or more mutations comprises a mutation of amino acid residue 212. In some embodiments, the one or more mutations comprises a mutation of amino acid residue 214. In some embodiments, the one or more mutations comprises a mutation of amino acid residue 222. In some embodiments, the one or more mutations comprises a mutation of amino acid residue 223. In some embodiments, the one or more mutations comprises a mutation of amino acid residue 224. In some embodiments, the one or more mutations comprises a mutation of amino acid residue 225. In some embodiments, the one or more mutations comprises a mutation of amino acid residue 226. In some embodiments, the one or more mutations comprises a mutation of amino acid residue 227. In some embodiments, the one or more mutations comprises a mutation of amino acid residue 228. In some embodiments, the one or more mutations comprises a mutation of amino acid residue 229. In some embodiments, the one or more mutations comprises a mutation of amino acid residue 230. In some embodiments, the one or more mutations comprises a mutation of amino acid residue 231. In some embodiments, the one or more mutations comprises a mutation of amino acid residue 232. In some embodiments, the one or more mutations comprises a mutation of amino acid residue 233. In some embodiments, the one or more mutations comprises a mutation of amino acid residue 243. In some embodiments, the one or more mutations comprises a mutation of amino acid residue 245. In some embodiments, the one or more mutations comprises a mutation of amino acid residue 248. In some embodiments, the one or more mutations comprises a mutation of amino acid residue 262.

In some embodiments, the nucleic acid molecule further comprises a sequence encoding one or more proteins or fragments thereof that inhibit an immune response by the complement system. In some embodiments, the one or more proteins or fragments thereof are selected from CD48, CD59, or a combination thereof. In some embodiments, the one or more proteins or fragments thereof is CD48. In some embodiments, the one or more proteins or fragments thereof is CD59. In some embodiments, the one or more proteins or fragments thereof are CD48 and CD59.

Provided herein, in another aspect, is a method for generating the nucleic acid molecule provided herein, comprising displacing a sequence encoding a region configured to receive a sequence comprising the deletion in the HLA locus, a sequence encoding the human HLA class 1 heavy chain sequence, or any combination thereof. In some embodiments, the method comprises displacing a sequence encoding a region configured to receive a sequence comprising the deletion in the HLA locus. In some embodiments, the method comprises displacing a sequence encoding a region configured to receive a sequence encoding the human HLA class 1 heavy chain sequence. In some embodiments, the method comprises displacing a sequence encoding a region configured to receive a sequence comprising the deletion in the HLA locus and a sequence encoding the human HLA class 1 heavy chain sequence.

### Immune Incompetent Cells

Provided herein, in another aspect, is a method of making an immune incompetent cell, comprising administering the complex provided herein or the nucleic acid molecule provided herein to a cell.

In some embodiments, the nucleic acid molecule is delivered to the cell's genome. In some embodiments, the cell is incubated with the complex.

In some embodiments, the cell is a stem cell. In some embodiments, the stem cell is an Induced Pluripotent stem cell (iPSC).

In some embodiments, the immune incompetent cells are suitable for use in cellular therapy. In some embodiments, the immune incompetent cells are suitable for administration to a subject without causing an immune response.

### Other Methods of Gene Therapy/Writing

### Vectors and Nucleic Acids

A variety of nucleic acids may be introduced into cells, for knockout purposes, or to obtain expression of a gene for other purposes. Nucleic acid constructs that can be used to produce transgenic cells including a target nucleic acid sequence. As used herein, the term nucleic acid or “nucleic acid molecule” includes DNA, RNA, and nucleic acid analogs, and nucleic acids that are double-stranded or single-stranded (i.e., a sense or an antisense single strand). Nucleic acid analogs can be modified at the base moiety, sugar moiety, or phosphate backbone to improve, for example, stability, hybridization, or solubility of the nucleic acid. Modifications at the base moiety include deoxyuridine for deoxythymidine, and 5-methyl-2′-deoxycytidine and 5-bromo-2′-doxycytidine for deoxycytidine. Modifications of the sugar moiety include modification of the 2′ hydroxyl of the ribose sugar to form 2′-O-methyl or 2′-O-allyl sugars. The deoxyribose phosphate backbone can be modified to produce morpholino nucleic acids, in which each base moiety is linked to a six membered, morpholino ring, or peptide nucleic acids, in which the deoxyphosphate backbone is replaced by a pseudopeptide backbone and the four bases are retained. See, Summerton and Weller (1997) Antisense Nucleic Acid Drug Dev. 7(3):187; and Hyrup et al. (1996) Bioorgan. Med. Chem. 4:5. In addition, the deoxyphosphate backbone can be replaced with, for example, a phosphorothioate or phosphorodithioate backbone, a phosphoroamidite, or an alkyl phosphotriester backbone.

The target nucleic acid sequence can be operably linked to a regulatory region such as a promoter. Regulatory regions can be from any species. As used herein, operably linked refers to positioning of a regulatory region relative to a nucleic acid sequence in such a way as to permit or facilitate transcription of the target nucleic acid.

Any type of promoter can be operably linked to a target nucleic acid sequence. Examples of promoters include, without limitation, tissue-specific promoters, constitutive promoters, and promoters responsive or unresponsive to a particular stimulus. Suitable tissue specific promoters can result in preferential expression of a nucleic acid transcript in beta cells and include, for example, the human insulin promoter. Other tissue specific promoters can result in preferential expression in, for example, hepatocytes or heart tissue and can include the albumin or alpha-myosin heavy chain promoters, respectively. In other embodiments, a promoter that facilitates the expression of a nucleic acid molecule without significant tissue- or temporal-specificity can be used (i.e., a constitutive promoter). For example, a beta-actin promoter such as the chicken beta-actin gene promoter, ubiquitin promoter, miniCAGs promoter, glyceraldehyde-3-phosphate dehydrogenase (GAPDH) promoter, or 3-phosphoglycerate kinase (PGK) promoter can be used, as well as viral promoters such as the herpes simplex virus thymidine kinase (HSV-TK) promoter, the SV40 promoter, or a cytomegalovirus (CMV) promoter. In some embodiments, a fusion of the chicken beta actin gene promoter and the CMV enhancer is used as a promoter. See, for example, Xu et al. (2001) Hum. Gene Ther. 12:563; and Kiwaki et al. (1996) Hum. Gene Ther. 7:821.

An example of an inducible promoter is the tetracycline (tet)-on promoter system, which can be used to regulate transcription of the nucleic acid. In this system, a mutated Tet repressor (TetR) is fused to the activation domain of herpes simplex virus VP 16 trans-activator protein to create a tetracycline-controlled transcriptional activator (tTA), which is regulated by tet or doxycycline (dox). In the absence of antibiotic, transcription is minimal, while in the presence of tet or dox, transcription is induced. Alternative inducible systems include the ecdysone or rapamycin systems. Ecdysone is an insect molting hormone whose production is controlled by a heterodimer of the ecdysone receptor and the product of the ultraspiracle gene (USP). Expression is induced by treatment with ecdysone or an analog of ecdysone such as muristerone A. The agent that is administered to the subject to trigger the inducible system is referred to as an induction agent.

Additional regulatory regions that may be useful in nucleic acid constructs, include, but are not limited to, polyadenylation sequences, translation control sequences (e.g., an internal ribosome entry segment, IRES), enhancers, inducible elements, or introns. Such regulatory regions may not be necessary, although they may increase expression by affecting transcription, stability of the mRNA, translational efficiency, or the like. Such regulatory regions can be included in a nucleic acid construct as desired to obtain optimal expression of the nucleic acids in the cell(s). Sufficient expression, however, can sometimes be obtained without such additional elements.

A nucleic acid construct may be used that encodes signal peptides or selectable markers. Signal peptides can be used such that an encoded polypeptide is directed to a particular cellular location (e.g., the cell surface). Non-limiting examples of selectable markers include puromycin, ganciclovir, adenosine deaminase (ADA), aminoglycoside phosphotransferase (neo, G418, APH), dihydrofolate reductase (DHFR), hygromycin-B-phosphtransferase, thymidine kinase (TK), and xanthin-guanine phosphoribosyltransferase (XGPRT). Such markers are useful for selecting stable transformants in culture. Other selectable markers include fluorescent polypeptides, such as green fluorescent protein or yellow fluorescent protein.

In some embodiments, a sequence encoding a selectable marker can be flanked by recognition sequences for a recombinase such as, e.g., Cre or Flp. For example, the selectable marker can be flanked by loxP recognition sites (34-bp recognition sites recognized by the Cre recombinase) or FRT recognition sites such that the selectable marker can be excised from the construct. See, Orban, et al., Proc. Natl. Acad. Sci. (1992) 89:6861, for a review of Cre/lox technology, and Brand and Dymecki, Dev. Cell (2004) 6:7. A transposon containing a Cre- or Flp-activatable transgene interrupted by a selectable marker gene also can be used to obtain transgenic cells with conditional expression of a transgene.

In some embodiments, the target nucleic acid encodes a polypeptide. A nucleic acid sequence encoding a polypeptide can include a tag sequence that encodes a “tag” designed to facilitate subsequent manipulation of the encoded polypeptide (e.g., to facilitate localization or detection). Tag sequences can be inserted in the nucleic acid sequence encoding the polypeptide such that the encoded tag is located at either the carboxyl or amino terminus of the polypeptide. Non-limiting examples of encoded tags include glutathione S transferase (GST) and FLAG™ tag (Kodak, New Haven, Conn.).

In other embodiments, the target nucleic acid sequence induces RNA interference against a target nucleic acid such that expression of the target nucleic acid is reduced. For example, the target nucleic acid sequence can induce RNA interference against a nucleic acid encoding a cystic fibrosis transmembrane conductance regulatory (CFTR) polypeptide. For example, double-stranded small interfering RNA (siRNA) or short hairpin RNA (shRNA) homologous to a CFTR DNA can be used to reduce expression of that DNA. Constructs for siRNA can be produced as described, for example, in Fire et al. (1998) Nature 391:806; Romano and Masino (1992) Mol. Microbiol. 6:3343; Cogoni et al. (1996) EMBO J. 15:3153; Cogoni and Masino (1999) Nature 399:166; Misquitta and Paterson (1999) Proc. Natl. Acad. Sci. USA 96:1451; and Kennerdell and Carthew (1998) Cell 95:1017. Constructs for shRNA can be produced as described by McIntyre and Fanning (2006) BMC Biotechnology 6:1. In general, shRNAs are transcribed as a single-stranded RNA molecule containing complementary regions, which can anneal and form short hairpins.

Nucleic acid constructs can be introduced into embryonic, fetal, or adult cells of any type, including, for example, germ cells such as an oocyte or an egg, a progenitor cell, an adult or embryonic stem cell, a hematopoietic stem cell, a mesenchymal stem cell, a primordial germ cell, a kidney cell such as a PK-15 cell, an islet cell, a beta cell, a liver cell, or a fibroblast such as a dermal fibroblast, using a variety of techniques. Non-limiting examples of techniques include the use of transposon systems, recombinant viruses that can infect cells, or liposomes or other non-viral methods such as electroporation, microinjection, or calcium phosphate precipitation, that are capable of delivering nucleic acids to cells.

In transposon systems, the transcriptional unit of a nucleic acid construct, i.e., the regulatory region operably linked to a target nucleic acid sequence, is flanked by an inverted repeat of a transposon. Several transposon systems, including, for example, Sleeping Beauty (see, U.S. Pat. No. 6,613,752 and U.S. Publication No. 2005/0003542); Frog Prince (Miskey et al. (2003) Nucleic Acids Res. 31:6873); To12 (Kawakami (2007) Genome Biology 8(Supp1.1):57; Minos (Pavlopoulos et al. (2007) Genome Biology 8(Supp1.1):S2); Hsmarl (Miskey et al. (2007)) Mol Cell Biol. 27:4589); and Passport have been developed to introduce nucleic acids into cells. The Sleeping Beauty and Passport transposon is particularly useful. A transposase can be delivered as a protein, encoded on the same nucleic acid construct as the target nucleic acid, can be introduced on a separate nucleic acid construct, or provided as an mRNA (e.g., an in vitro-transcribed and capped mRNA).

Insulator elements also can be included in a nucleic acid construct to maintain expression of the target nucleic acid and to inhibit the unwanted transcription of host genes. See, for example, U.S. Publication No. 2004/0203158. Typically, an insulator element flanks each side of the transcriptional unit and is internal to the inverted repeat of the transposon. Non-limiting examples of insulator elements include the matrix attachment region-(MAR) type insulator elements and border-type insulator elements. See, for example, U.S. Pat. Nos. 6,395,549, 5,731,178, 6,100,448 and 5,610,053, and U.S. Publication No. 2004/0203158.

Nucleic acids can be incorporated into vectors. A vector is a broad term that includes any specific DNA segment that is designed to move from a carrier into a target DNA. A vector may be referred to as an expression vector, or a vector system, which is a set of components needed to bring about DNA insertion into a genome or other targeted DNA sequence such as an episome, plasmid, or even virus/phage DNA segment. Vector systems such as viral vectors (e.g., retroviruses, adeno-associated virus and integrating phage viruses), and non-viral vectors (e.g., transposons) used for gene delivery in subjects have two basic components: 1) a vector comprised of DNA (or RNA that is reverse transcribed into a cDNA) and 2) a transposase, recombinase, or other integrase enzyme that recognizes both the vector and a DNA target sequence and inserts the vector into the target DNA sequence. Vectors most often contain one or more expression cassettes that comprise one or more expression control sequences, wherein an expression control sequence is a DNA sequence that controls and regulates the transcription and/or translation of another DNA sequence or mRNA, respectively.

Many different types of vectors are known. For example, plasmids and viral vectors, e.g., retroviral vectors, are known. Mammalian expression plasmids typically have an origin of replication, a suitable promoter and optional enhancer, and also any necessary ribosome binding sites, a polyadenylation site, splice donor and acceptor sites, transcriptional termination sequences, and 5′ flanking non-transcribed sequences. Examples of vectors include: plasmids (which may also be a carrier of another type of vector), adenovirus, adeno-associated virus (AAV), lentivirus (e.g., HIV-1, SIV or FIV), retrovirus (e.g., ASV, ALV or MoMLV), and transposons (e.g., Sleeping Beauty, P-elements, Tol-2, Frog Prince, piggyBac).

Provided herein, in other aspects, are additional methods of delivering a nucleic acid molecule encoding one or more human leukocyte antigens (HLAs) to a cell. In some embodiments, the method comprises delivery of a nucleic acid molecule encoding one or more HLAs via a viral vector. In some embodiments, the method comprises delivery of a nucleic acid molecule encoding one or more HLAs via a non-viral vector. In some embodiments, the viral vector is derived from a lentivirus.

In some embodiments, the nucleic acid molecule comprises a deletion in the endogenous HLA locus. In some embodiments, the deletion comprises a deletion in the endogenous HLA-A, HLA-B, or HLA-C locus, or any combination thereof. In some embodiments, the deletion is complete deletion of the endogenous HLA locus.

In some embodiments, the nucleic acid molecule further comprises a sequence encoding a human HLA class 1 heavy chain sequence. In some embodiments, the sequence encoding a human HLA class 1 heavy chain sequence comprises an HLA-A sequence, an HLA-B sequence, an HLA-C sequence, or any combination thereof. In some embodiments, the sequence encoding a human HLA class 1 heavy chain sequence comprises multiple alleles of an HLA-A sequence, an HLA-B sequence, an HLA-C sequence, or any combination thereof. In some embodiments, the sequence encoding a human HLA class 1 heavy chain sequence comprises an HLA-A sequence, wherein the HLA-A sequence is displaced between the HLA-B sequence and the HLA-C sequence.

In some embodiments, the sequence encoding a human HLA class 1 heavy chain sequence comprises fewer than 1700 base pairs (bp). In some embodiments, the sequence encoding a human HLA class 1 heavy chain sequence comprises fewer than 500 bp. In some embodiments, the sequence encoding a human HLA class 1 heavy chain sequence comprises fewer than 250 bp. In some embodiments, the sequence encoding a human HLA class 1 heavy chain sequence comprises fewer than 150 bp.

In some embodiments, the HLA-A sequence, HLA-B sequence, HLA-C sequences, or combination thereof comprises one or more flanking sequences. In some embodiments, the one or more flanking sequences comprise an endogenous HLA sequence. In some embodiments, the one or more flanking sequences are specific to one or more promoters. In some embodiments, the promoters comprise an HLA-A promoter, HLA-B promoter, HLA-C promoter, or combination thereof.

In some embodiments, the sequence encoding a human HLA class 1 heavy chain sequence does not comprise at least a portion of the HLA-A sequence, HLA-B sequence, HLA-C sequence, or combination thereof.

In some embodiments, the nucleic acid molecule encoding the human HLA class 1 heavy chain sequence comprises an HLA-E sequence or a fragment thereof, an HLA-F sequence or a fragment thereof, an HLA-G sequence or a fragment thereof, or any combination thereof. In some embodiments, at least one of the HLA-E sequence or the fragment thereof, HLA-F sequence or the fragment thereof, HLA-G sequence or the fragment thereof, or any combination thereof is inhibited from eliciting a T-cell response when the complex is interrogated by one or more T-cells.

In some embodiments, the nucleic acid molecule encoding a human HLA class 1 heavy chain sequence comprises one or more mutations, wherein a cell comprising the mutated human HLA class 1 heavy chain sequence comprising the one or more mutations does not elicit an immune response when the cell is interrogated by one or more CD8 cells. In some embodiments, the mutated human HLA class 1 heavy chain sequence encodes an HLA comprising one or more mutations at one or more of amino acid residues 115, 122, 128, 194, 197, 198, 212, 214, 222, 223, 224, 225, 226, 227, 228, 229, 230, 231, 232, 233, 243, 245, 248, 262, or any combination thereof.

### Templated and Non-Templated Repairs

Targeted endonuclease technologies, such as zinc finger nucleases (ZFNs), TAL effector nucleases (TALENs) and clustered regularly interspaced short palindromic repeats/CRISPR associated endonuclease cas9 (CRISPR/Cas9) can be utilized to disrupt gene function by introducing insertions and/or deletions (indels) into genomes of species, such as by non-homologous end-joining (NHEJ). However, indels introduced by NHEJ are variable in size and sequence which makes screening for functionally disrupted clones arduous and does not enable precise alterations. TALEN or CRISPR/Cas9 mediated homology-directed repair (HDR) supports the introduction of defined nucleotide changes in eukaryotic cells.

A subject may be modified using TALENs, zinc finger nucleases, or other genetic engineering tools, including various vectors that are known. A genetic modification made by such tools may comprise inactivation of a gene. The term inactivation of a gene refers to preventing the formation of a functional gene product. A gene product is functional only if it fulfills its normal (wild-type) functions. Materials and methods of genetically modifying subjects are further detailed in U.S. Ser. No. 13/404,662 filed Feb. 24, 2012, Ser. No. 13/467,588 filed May 9, 2012, and Ser. No. 12/622,886 filed Nov. 10, 2009 which are hereby incorporated herein by reference for all purposes; in case of conflict, the instant specification is controlling. The term trans-acting refers to processes acting on a target gene from a different molecule (i.e., intermolecular). A trans-acting element is usually a DNA sequence that contains a gene. This gene codes for a protein (or microRNA or other diffusible molecule) that is used in the regulation of the target gene. The trans-acting gene may be on the same chromosome as the target gene, but the activity is via the intermediary protein or RNA that it encodes. Inactivation of a gene using a dominant negative generally involves a trans-acting element. The term cis-regulatory or cis-acting means an action without coding for protein or RNA; in the context of gene inactivation, this generally means inactivation of the coding portion of a gene, or a promoter and/or operator that is necessary for expression of the functional gene.

Various techniques known in the art can be used to introduce nucleic acid constructs into non-humans and humans to produce founder lines, in which the nucleic acid construct is integrated into the genome. Such techniques include, without limitation, pronuclear microinjection (U.S. Pat. No. 4,873,191), retrovirus mediated gene transfer into germ lines (Van der Putten et al. (1985) Proc. Natl. Acad. Sci. USA 82, 6148-1652), gene targeting into embryonic stem cells (Thompson et al. (1989) Cell 56, 313-321), electroporation of embryos (Lo (1983) Mol. Cell. Biol. 3, 1803-1814), sperm-mediated gene transfer (Lavitrano et al. (2002) Proc. Natl. Acad. Sci. USA 99, 14230-14235; Lavitrano et al. (2006) Reprod. Fert. Develop. 18, 19-23), and in vitro transformation of somatic cells, such as cumulus or mammary cells, or adult, fetal, or embryonic stem cells, followed by nuclear transplantation (Wilmut et al. (1997) Nature 385, 810-813; and Wakayama et al. (1998) Nature 394, 369-374). Pronuclear microinjection, sperm mediated gene transfer, and somatic cell nuclear transfer are particularly useful techniques, as well as cytoplasmic injection, primordial germ cell transplantation (Brinster), and blastocyst chimera production whereby a germ cell is propagated in an embryo.

TALENs, zinc finger nucleases, CRISPR nuclease (e.g., CRISPR/Cas9) and recombinase fusion proteins may be used with or without a template. A template is an exogenous DNA added to the cell for cellular repair machinery to use as a guide (template) to repair double stranded breaks (DSB) in DNA. This process is generally referred to as homology directed repair (HDR). Processes without a template involve making DSBs and providing for cellular machinery to make repairs that are often less than perfect, so that an insertion or deletion (an indel) is made. The cellular pathway referred to as non-homologous end joining (NHEJ) typically mediates non-templated repairs of DSBs. The term NHEJ is commonly used to refer to all such non-templated repairs regardless of whether the NHEJ was involved, or an alternative cellular pathway.

### Targeted Nuclease Systems

Genome editing tools such as transcription activator-like effector nucleases (TALENs) and zinc finger nucleases (ZFNs) have impacted the fields of biotechnology, gene therapy and functional genomic studies in many organisms. More recently, RNA-guided endonucleases (RGENs) are directed to their target sites by a complementary RNA molecule. The Cas9/CRISPR system is a RGEN. tracrRNA is another such tool. These are examples of targeted nuclease systems: these system have a DNA-binding member that localizes the nuclease to a target site. The site is then cut by the nuclease. TALENs and ZFNs have the nuclease fused to the DNA-binding member. Cas9/CRISPR are cognates that find each other on the target DNA. The DNA-binding member has a cognate sequence in the chromosomal DNA. The DNA-binding member is typically designed in light of the intended cognate sequence so as to obtain a nucleolytic action at nor near an intended site. Certain embodiments are applicable to all such systems without limitation; including, embodiments that minimize nuclease re-cleavage, embodiments for making SNPs with precision at an intended residue, embodiments for making indels with precision at an intended residue and placement of the allele that is being introgressed at the DNA-binding site.

### Zinc Finger Nucleases (ZFNs)

Zinc-finger nucleases (ZFNs) are artificial restriction enzymes generated by fusing a zinc finger DNA-binding domain to a DNA-cleavage domain. Zinc finger domains can be engineered to target desired DNA sequences and this enables zinc-finger nucleases to target unique sequences within complex genomes. By taking advantage of endogenous DNA repair machinery, these reagents can be used to alter the genomes of higher organisms. ZFNs may be used in methods for inactivating genes.

A zinc finger DNA-binding domain has about 30 amino acids and folds into a stable structure. Each finger primarily binds to a triplet within the DNA substrate. Amino acid residues at key positions contribute to most of the sequence-specific interactions with the DNA site. These amino acids can be changed while maintaining the remaining amino acids to preserve the necessary structure. Binding to longer DNA sequences is achieved by linking several domains in tandem. Other functionalities like non-specific FokI cleavage domain (N), transcription activator domains (A), transcription repressor domains (R) and methylases (M) can be fused to a ZFPs to form ZFNs respectively, zinc finger transcription activators (ZFA), zinc finger transcription repressors (ZFR, and zinc finger methylases (ZFM).

### Transcription Activator-Like Effector Nucleases (TALENs)

The term TALEN, as used herein, is broad and includes a monomeric TALEN that can cleave double stranded DNA without assistance from another TALEN, e.g., as in Beurdeley, M. et al. Compact designer TALENs for efficient genome engineering. Nat. Commun. 4:1762 doi: 10.1038/ncomms2782 (2013). The term TALEN is also used to refer to one or both members of a pair of TALENs that are engineered to work together to cleave DNA at the same site. TALENs that work together may be referred to as a left-TALEN and a right-TALEN, which references the handedness of DNA or a TALEN-pair.

In some embodiments, a monomeric TALEN can be used. TALENs typically function as dimers across a bipartite recognition site with a spacer, such that two TAL effector domains are each fused to a catalytic domain of the FokI restriction enzyme, the DNA-recognition sites for each resulting TALEN are separated by a spacer sequence, and binding of each TALEN monomer to the recognition site allows FokI to dimerize and create a double-strand break within the spacer. Monomeric TALENs also can be constructed, however, such that single TAL effectors are fused to a nuclease that does not require dimerization to function. One such nuclease, for example, is a single-chain variant of FokI in which the two monomers are expressed as a single polypeptide. Other naturally occurring or engineered monomeric nucleases also can serve this role. The DNA recognition domain used for a monomeric TALEN can be derived from a naturally occurring TAL effector. Alternatively, the DNA recognition domain can be engineered to recognize a specific DNA target. Engineered single-chain TALENs may be easier to construct and deploy, as they require only one engineered DNA recognition domain. A dimeric DNA sequence-specific nuclease can be generated using two different DNA binding domains (e.g., one TAL effector binding domain and one binding domain from another type of molecule). TALENs may function as dimers across a bipartite recognition site with a spacer. This nuclease architecture also can be used for target-specific nucleases generated from, for example, one TALEN monomer and one zinc finger nuclease monomer. In such cases, the DNA recognition sites for the TALEN and zinc finger nuclease monomers can be separated by a spacer of appropriate length. Binding of the two monomers can allow FokI to dimerize and create a double-strand break within the spacer sequence. DNA binding domains other than zinc fingers, such as homeodomains, myb repeats or leucine zippers, also can be fused to FokI and serve as a partner with a TALEN monomer to create a functional nuclease.

In some embodiments, a TAL effector can be used to target other protein domains (e.g., non-nuclease protein domains) to specific nucleotide sequences. For example, a TAL effector can be linked to a protein domain from, without limitation, a DNA 20 interacting enzyme (e.g., a methylase, a topoisomerase, an integrase, a transposase, or a ligase), a transcription activators or repressor, or a protein that interacts with or modifies other proteins such as histones. Applications of such TAL effector fusions include, for example, creating or modifying epigenetic regulatory elements, making site-specific insertions, deletions, or repairs in DNA, controlling gene expression, and modifying chromatin structure.

The spacer of the target sequence can be selected or varied to modulate TALEN specificity and activity. The flexibility in spacer length indicates that spacer length can be chosen to target particular sequences with high specificity. Further, the variation in activity has been observed for different spacer lengths indicating that spacer length can be chosen to achieve a desired level of TALEN activity.

Alternative embodiments use alternative mRNA polymerases and cognate binding sites such as T7 or SP6. Other embodiments relate to the use of any of several alterations of the UTR sequences; these could benefit translation of the mRNA. Some examples are: addition of a cytoplasmic polyadenylation element binding site in the 3′ UTR, or exchanging the Xenopus β-globin UTRs with UTR sequences from human, pig, cow, sheep, goat, zebrafish, from genes including B-globin. UTRs from genes may be selected for regulation of expression in embryonic development or in cells. Some examples of UTRs that may be useful include β-actin, DEAH (SEQ ID NO: 527), TPT1, ZF42, SKP1, TKT, TP3, DDXS, EIF3A, DDX39, GAPDH, CDK1, Hsp90ab1, Ybx1 fEif4b Rps27a Stra13, Myc, Paf1 and Foxo1, or CHUK. Such vector or mRNA improvements could be used to direct special or temporal expression of ectopic TALENs for study of gene depletion at desired stages of development.

In some embodiments, a monomeric TALEN can be used. TALEN typically function as dimers across a bipartite recognition site with a spacer, such that two TAL effector domains are each fused to a catalytic domain of the FokI restriction enzyme, the DNA-recognition sites for each resulting TALEN are separated by a spacer sequence, and binding of each TALEN monomer to the recognition site allows FokI to dimerize and create a double-strand break within the spacer. Monomeric TALENs also can be constructed, however, such that single TAL effectors are fused to a nuclease that does not require dimerization to function. One such nuclease, for example, is a single-chain variant of FokI in which the two monomers are expressed as a single polypeptide. Other naturally occurring or engineered monomeric nucleases also can serve this role. The DNA recognition domain used for a monomeric TALEN can be derived from a naturally occurring TAL effector. Alternatively, the DNA recognition domain can be engineered to recognize a specific DNA target. Engineered single-chain TALENs may be easier to construct and deploy, as they require only one engineered DNA recognition domain. A dimeric DNA sequence-specific nuclease can be generated using two different DNA binding domains (e.g., one TAL effector binding domain and one binding domain from another type of molecule). TALENs may function as dimers across a bipartite recognition site with a spacer. This nuclease architecture also can be used for target-specific nucleases generated from, for example, one TALEN monomer and one zinc finger nuclease monomer. In such cases, the DNA recognition sites for the TALEN and zinc finger nuclease monomers can be separated by a spacer of appropriate length. Binding of the two monomers can allow FokI to dimerize and create a double-strand break within the spacer sequence. DNA binding domains other than zinc fingers, such as homeodomains, myb repeats or leucine zippers, also can be fused to FokI and serve as a partner with a TALEN monomer to create a functional nuclease.

The term nuclease includes exonucleases and endonucleases. The term endonuclease refers to any wild-type or variant enzyme capable of catalyzing the hydrolysis (cleavage) of bonds between nucleic acids within a DNA or RNA molecule, preferably a DNA molecule. Non-limiting examples of endonucleases include type II restriction endonucleases such as FokI, HhaI, HindIII, NotI, BbvCl, EcoRI, BglII, and AhwI. Endonucleases comprise also rare-cutting endonucleases when having typically a polynucleotide recognition site of about 12-45 basepairs (bp) in length, more preferably of 14-45 bp. Rare-cutting endonucleases induce DNA double-strand breaks (DSBs) at a defined locus. Rare-cutting endonucleases can for example be a homing endonuclease, a chimeric Zinc-Finger nuclease (ZFN) resulting from the fusion of engineered zinc-finger domains with the catalytic domain of a restriction enzyme such as FokI or a chemical endonuclease. In chemical endonucleases, a chemical or peptidic cleaver is conjugated either to a polymer of nucleic acids or to another DNA recognizing a specific target sequence, thereby targeting the cleavage activity to a specific sequence. Chemical endonucleases also encompass synthetic nucleases like conjugates of orthophenanthroline, a DNA cleaving molecule, and triplex-forming oligonucleotides (TFOs), known to bind specific DNA sequences. Such chemical endonucleases are comprised in the term “endonuclease” according to the present invention. Examples of such endonuclease include I-See I, I-Chu L I-Cre I, I-Csm I, PI-See L PI-Tti L PI-Mtu I, I-Ceu I, I-See IL 1-See III, HO, PI-Civ I, PI-Ctr L PI-Aae I, PI-Bsu I, PI-Dha I, PI-Dra L PI-May L PI-Meh I, PI-Mfu L PI-Mfl I, PI-Mga L PI-Mgo I, PI-Min L PI-Mka L PI-Mle I, PI-Mma I, PI-30 Msh L PI-Msm I, PI-Mth I, PI-Mtu I, PI-Mxe I, PI-Npu I, PI-Pfu L PI-Rma I, PI-Spb I, PI-Ssp L PI-Fae L PI-Mja I, PI-Pho L PI-Tag L PI-Thy I, PI-Tko I, PI-Tsp I, I-Msol.

A genetic modification made by TALENs or other tools may be, for example, chosen from the list consisting of an insertion, a deletion, insertion of an exogenous nucleic acid fragment, and a substitution. The term “insertion” is used broadly to mean either literal insertion into the chromosome or use of the exogenous sequence as a template for repair. In general, a target DNA site is identified and a TALEN-pair is created that will specifically bind to the site. The TALEN is delivered to the cell or embryo, e.g., as a protein, mRNA or by a vector that encodes the TALEN. The TALEN cleaves the DNA to make a double-strand break that is then repaired, often resulting in the creation of an indel, or incorporating sequences or polymorphisms contained in an accompanying exogenous nucleic acid that is either inserted into the chromosome or serves as a template for repair of the break with a modified sequence. This template-driven repair is a useful process for changing a chromosome, and provides for effective changes to cellular chromosomes.

The term exogenous nucleic acid means a nucleic acid that is added to the cell or embryo, regardless of whether the nucleic acid is the same or distinct from nucleic acid sequences naturally in the cell. In some cases, the exogenous nucleic acid differs in sequence from any nucleic acid sequence that occurs naturally within the cell. The term nucleic acid fragment is broad and includes a chromosome, expression cassette, gene, DNA, RNA, mRNA, or portion thereof.

Genetic modification of cells may also include insertion of a reporter. The reporter may be, e.g., a florescent marker, e.g., green fluorescent protein and yellow fluorescent protein. The reporter may be a selection marker, e.g., puromycin, ganciclovir, adenosine deaminase (ADA), aminoglycoside phosphotransferase (neo, G418, APH), dihydrofolate reductase (DHFR), hygromycin-B-phosphtransferase, thymidine kinase (TK), or xanthin-guanine phosphoribosyltransferase (XGPRT). Vectors for the reporter, selection marker, and/or one or more TALEN may be a plasmid,

TALENs may be directed to a plurality of DNA sites. The sites may be separated by several thousand or many thousands of base pairs. The DNA can be rejoined by cellular machinery to thereby cause the deletion of the entire region between the sites. Embodiments include, for example, sites separated by a distance between 1-5 megabases or between 50% and 80% of a chromosome, or between about 100 and about 1,000,000 basepairs; artisans will immediately appreciate that all the ranges and values within the explicitly stated ranges are contemplated, e.g., from about 1,000 to about 10,000 basepairs or from about 500 to about 500,000 basepairs. Alternatively, exogenous DNA may be added to the cell or embryo for insertion of the exogenous DNA, or template-driven repair of the DNA between the sites. Modification at a plurality of sites may be used to make genetically modified cells, embryos, artiodactyls, and livestock. One or more genes may be chosen for complete or at least partial deletion, including a sexual maturation gene or a cis-acting factor thereof.

### Recombinases

Embodiments of the invention include administration of a TALEN or TALENs with a recombinase or other DNA-binding protein associated with DNA recombination. A recombinase forms a filament with a nucleic acid fragment and, in effect, searches cellular DNA to find a DNA sequence substantially homologous to the sequence. An embodiment of a TALEN-recombinase embodiment comprises combining a recombinase with a nucleic acid sequence that serves as a template for HDR. The HDR template sequence has substantial homology to a site that is targeted for cutting by the TALEN/TALEN pair. As described herein, the HDR template provides for a change to the native DNA, by placement of an allele, creation of an indel, insertion of exogenous DNA, or with other changes. The TALEN is placed in the cell or embryo by methods described herein as a protein, mRNA, or by use of a vector. The recombinase is combined with the HDR template to form a filament and placed into the cell. The recombinase and/or HDR template that combines with the recombinase may be placed in the cell or embryo as a protein, an mRNA, or with a vector that encodes the recombinase. The term recombinase refers to a genetic recombination enzyme that enzymatically catalyzes, in a cell, the joining of relatively short pieces of DNA between two relatively longer DNA strands. Recombinases include Cre recombinase, Hin recombinase, RecA, RAD51, Cre, and FLP. Cre recombinase is a Type I topoisomerase from P1 bacteriophage that catalyzes site-specific recombination of DNA between loxP sites. Hin recombinase is a 21 kD protein composed of 198 amino acids that is found in the bacteria Salmonella. Hin belongs to the serine recombinase family of DNA invertases in which it relies on the active site serine to initiate DNA cleavage and recombination. RAD51 is a human gene. The protein encoded by this gene is a member of the RAD51 protein family which assist in repair of DNA double strand breaks. RAD51 family members are homologous to the bacterial RecA and yeast Rad51 genes. Cre recombinase is an enzyme that is used in experiments to delete specific sequences that are flanked by loxP sites. FLP refers to Flippase recombination enzyme (FLP or Flp) derived from the 21.1, plasmid of the baker's yeast Saccharomyces cerevisiae.

Herein, “RecA” or “RecA protein” refers to a family of RecA-like recombination proteins having essentially all or most of the same functions, particularly: (i) the ability to position properly oligonucleotides or polynucleotides on their homologous targets for subsequent extension by DNA polymerases; (ii) the ability topologically to prepare duplex nucleic acid for DNA synthesis; and, (iii) the ability of RecA/oligonucleotide or RecA/polynucleotide complexes efficiently to find and bind to complementary sequences. The best characterized RecA protein is from E. coli; in addition to the original allelic form of the protein a number of mutant RecA-like proteins have been identified, for example, RecA803. Further, many organisms have RecA-like strand-transfer proteins including, for example, yeast, Drosophila, mammals including humans, and plants. These proteins include, for example, Red, Rec2, Rad51, Rad51B, Rad51C, Rad51D, Rad51E, XRCC2 and DMC1. An embodiment of the recombination protein is the RecA protein of E. coli. Alternatively, the RecA protein can be the mutant RecA-803 protein of E. coli, a RecA protein from another bacterial source or a homologous recombination protein from another organism.

RecA is known for its recombinase activity to catalyze strand exchange during the repair of double-strand breaks by homologous recombination (McGrew and Knight, 2003) Radding, et al., 1981; Seitz et al., 1998). RecA has also been shown to catalyze proteolysis, e.g., of the LexA and X repressor proteins, and to possess DNA-dependent ATPase activity. After a double-strand break occurs from ionizing radiation or some other insult, exonucleases chew back the DNA ends 5′ to 3′, thereby exposing one strand of the DNA (Cox, 1999; McGrew and Knight, 2003). The single-stranded DNA becomes stabilized by single-strand binding protein (SSB). After binding of SSB, RecA binds the single-stranded (ss) DNA and forms a helical nucleoprotein filament (referred to as a filament or a presynaptic filament). During DNA repair, the homology-searching functions of RecA direct the filament to homologous DNA and catalyze homologous base pairing and strand exchange. This results in the formation of DNA heteroduplex. After strand invasion, DNA polymerase elongates the ssDNA based on the homologous DNA template to repair the DNA break, and crossover structures or Holliday junctions are formed. RecA also shows a motor function that participates in the migration of the crossover structures (Campbell and Davis, 1999).

Recombinase activity comprises a number of different functions. For example, polypeptide sequences having recombinase activity are able to bind in a non-sequence-specific fashion to single-stranded DNA to form a nucleoprotein filament. Such recombinase-bound nucleoprotein filaments are able to interact in a non-sequence-specific manner with a double-stranded DNA molecule, search for sequences in the double-stranded molecule that are homologous to sequences in the filament, and, when such sequences are found, displace one of the strands of the double-stranded molecule to allow base-pairing between sequences in the filament and complementary sequences in one of the strands of the double stranded molecule. Such steps are collectively denoted “synapsis.”

Thus recombinase activities include, but are not limited to, single-stranded DNA-binding, synapsis, homology searching, duplex invasion by single-stranded DNA, heteroduplex formation, ATP hydrolysis and proteolysis. The prototypical recombinase is the RecA protein from E. coli. See, for example, U.S. Pat. No. 4,888,274. Prokaryotic RecA-like proteins have also been described in Salmonella, Bacillus and Proteus species. A thermostable RecA protein, from Thermus aquaticus, has been described in U.S. Pat. No. 5,510,473. A bacteriophage T4 homologue of RecA, the UvsX protein, has been described. RecA mutants, having altered recombinase activities, have been described, for example, in U.S. Pat. Nos. 6,774,213; 7,176,007 and 7,294,494. Plant RecA homologues are described in, for example, U.S. Pat. Nos. 5,674,992; 6,388,169 and 6,809,183. RecA fragments containing recombinase activity have been described, for example, in U.S. Pat. No. 5,731,411. Mutant RecA proteins having enhanced recombinase activity such as, for example, RecA803 have been described. See, for example, Madiraju et al. (1988) Proc. Natl. Acad. Sci. USA 85:6592-6596.

A eukaryotic homologue of RecA, also possessing recombinase activity, is the Rad51 protein, first identified in the yeast Saccharomyces cerevisiae. See Bishop et al., (1992) Cell 69:439-56; Shinohara et al, (1992) Cell: 457-70; Aboussekhra, et al., (1992) Mol. Cell. Biol. 72, 3224-3234 and Basile et al., (1992) Mol. Cell. Biol. 12, 3235-3246. Plant Rad51 sequences are described in U.S. Pat. Nos. 6,541,684; 6,720,478; 6,905,857 and 7,034,117. Another yeast protein that is homologous to RecA is the Dmcl protein. RecA/Rad51 homologues in organisms other than E. coli and S. cerevisiae have been described. Morita et al. (1993) Proc. Natl. Acad. Sci. USA 90:6577-6580; Shinohara et al. (1993) Nature Genet. 4:239-243; Heyer (1994) Experientia 50:223-233; Maeshima et al. (1995) Gene 160:195-200; U.S. Pat. Nos. 6,541,684 and 6,905,857.

Additional descriptions of proteins having recombinase activity are found, for example, in Fugisawa et al. (1985) Nucl. Acids Res. 13:7473; Hsieh et al. (1986) Cell 44:885; Hsieh et al. (1989) J. Biol. Chem. 264:5089; Fishel et al. (1988) Proc. Natl. Acad. Sci. USA 85:3683; Cassuto et al. (1987) Mol. Gen. Genet. 208:10; Ganea et al. (1987) Mol. Cell Biol. 7:3124; Moore et al. (1990) J. Biol. Chem.:11108; Keene et al. (1984) Nucl. Acids Res. 12:3057; Kimiec (1984) Cold Spring Harbor Symp. 48:675; Kimeic (1986) Cell 44:545; Kolodner et al. (1987) Proc. Natl. Acad. Sci. USA 84:5560; Sugino et al. (1985) Proc. Natl. Acad, Sci. USA 85: 3683; Halbrook et al. (1989) J. Biol. Chem. 264:21403; Eisen et al. (1988) Proc. Natl. Acad. Sci. USA 85:7481; McCarthy et al. (1988) Proc. Natl. Acad. Sci. USA 85:5854; and Lowenhaupt et al. (1989) J. Biol. Chem. 264:20568, which are incorporated herein by reference. See also Brendel et al. (1997) J. Mol. Evol. 44:528.

Examples of proteins having recombinase activity include recA, recA803, uvsX, and other recA mutants and recA-like recombinases (Roca (1990) Crit. Rev. Biochem. Molec. Biol. 25:415), (Kolodner et al. (1987) Proc. Natl. Acad. Sci. U.S.A. 84:5560; Tishkoff et al. (1991) Molec. Cell. Biol. 11:2593), RuvC (Dunderdale et al. (1991) Nature 354:506), DST2, KEM1 and XRN1 (Dykstra et al. (1991) Molec. Cell. Biol. 11:2583), STPa/DST1 (Clark et al. (1991) Molec. Cell. Biol. 11:2576), HPP-1 (Moore et al. (1991) Proc. Natl. Acad. Sci. U.S.A. 88:9067), other eukaryotic recombinases (Bishop et al. (1992) Cell 69:439; and Shinohara et al. (1992) Cell 69:457); incorporated herein by reference.

In vitro-evolved proteins having recombinase activity have been described in U.S. Pat. No. 6,686,515. Further publications relating to recombinases include, for example, U.S. Pat. Nos. 7,732,585, 7,361,641, 7,144,734. For a review of recombinases, see Cox (2001) Proc. Natl. Acad. Sci. USA 98:8173-8180.

A nucleoprotein filament, or “filament” may be formed. The term filament, in the context of forming a structure with a recombinase, is a term known to artisans in these fields. The nucleoprotein filament so formed can then be, e.g., contacted with another nucleic acid or introduced into a cell. Methods for forming nucleoprotein filaments, wherein the filaments comprise polypeptide sequences having recombinase activity and a nucleic acid, are well-known in the art. See, e.g., Cui et al. (2003) Marine Biotechnol. 5:174-184 and U.S. Pat. Nos. 4,888,274; 5,763,240; 5,948,653 and 7,199,281, the disclosures of which are incorporated by reference for the purposes of disclosing exemplary techniques for binding recombinases to nucleic acids to form nucleoprotein filaments.

In general, a molecule having recombinase activity is contacted with a linear, single-stranded nucleic acid. The linear, single-stranded nucleic acid may be a probe. The methods of preparation of such single stranded nucleic acids are known. The reaction mixture typically contains a magnesium ion. Optionally, the reaction mixture is buffered and optionally also contains ATP, dATP or a nonhydrolyzable ATP analogue, such as, for example, γ-thio-ATP (ATP-γ-S) or γ-thio-GTP (GTP-γ-S). Reaction mixtures can also optionally contain an ATP-generating system. Double-stranded DNA molecules can be denatured (e.g., by heat or alkali) either prior to, or during, filament formation. Optimization of the molar ratio of recombinase to nucleic acid is within the skill of the art. For example, a series of different concentrations of recombinase can be added to a constant amount of nucleic acid, and filament formation assayed by mobility in an agarose or acrylamide gel. Because bound protein retards the electrophoretic mobility of a polynucleotide, filament formation is evidenced by retarded mobility of the nucleic acid. Either maximum degree of retardation, or maximum amount of nucleic acid migrating with a retarded mobility, can be used to indicate optimal recombinase:nucleic acid ratios. Protein-DNA association can also be quantitated by measuring the ability of a polynucleotide to bind to nitrocellulose.

Provided herein, in other aspects, are additional methods of delivering a nucleic acid molecule encoding one or more human leukocyte antigens (HLAs) to a cell. In some embodiments, the method comprises delivery of a nucleic acid molecule encoding one or more HLAs via transcription activator-like effector nucleases (TALENs). In some embodiments, the method comprises delivery of a nucleic acid molecule encoding one or more HLAs via zinc finger nucleases (ZFNs). In some embodiments, the method comprises delivery of a nucleic acid molecule encoding one or more HLAs via clustered regularly interspaced short palindromic repeats/CRISPR associated endonuclease cas9 (CRISPR/Cas9).

In some embodiments, the nucleic acid molecule comprises a deletion in the endogenous HLA locus. In some embodiments, the deletion comprises a deletion in the endogenous HLA-A, HLA-B, or HLA-C locus, or any combination thereof. In some embodiments, the deletion is complete deletion of the endogenous HLA locus.

In some embodiments, the nucleic acid molecule further comprises a sequence encoding a human HLA class 1 heavy chain sequence. In some embodiments, the sequence encoding a human HLA class 1 heavy chain sequence comprises an HLA-A sequence, an HLA-B sequence, an HLA-C sequence, or any combination thereof. In some embodiments, the sequence encoding a human HLA class 1 heavy chain sequence comprises multiple alleles of an HLA-A sequence, an HLA-B sequence, an HLA-C sequence, or any combination thereof. In some embodiments, the sequence encoding a human HLA class 1 heavy chain sequence comprises an HLA-A sequence, wherein the HLA-A sequence is displaced between the HLA-B sequence and the HLA-C sequence.

In some embodiments, the sequence encoding a human HLA class 1 heavy chain sequence comprises fewer than 1700 base pairs (bp). In some embodiments, the sequence encoding a human HLA class 1 heavy chain sequence comprises fewer than 500 bp. In some embodiments, the sequence encoding a human HLA class 1 heavy chain sequence comprises fewer than 250 bp. In some embodiments, the sequence encoding a human HLA class 1 heavy chain sequence comprises fewer than 150 bp.

In some embodiments, the HLA-A sequence, HLA-B sequence, HLA-C sequences, or combination thereof comprises one or more flanking sequences. In some embodiments, the one or more flanking sequences comprise an endogenous HLA sequence. In some embodiments, the one or more flanking sequences are specific to one or more promoters. In some embodiments, the promoters comprise an HLA-A promoter, HLA-B promoter, HLA-C promoter, or combination thereof.

In some embodiments, the sequence encoding a human HLA class 1 heavy chain sequence does not comprise at least a portion of the HLA-A sequence, HLA-B sequence, HLA-C sequence, or combination thereof.

In some embodiments, the nucleic acid molecule encoding the human HLA class 1 heavy chain sequence comprises an HLA-E sequence or a fragment thereof, an HLA-F sequence or a fragment thereof, an HLA-G sequence or a fragment thereof, or any combination thereof. In some embodiments, at least one of the HLA-E sequence or the fragment thereof, HLA-F sequence or the fragment thereof, HLA-G sequence or the fragment thereof, or any combination thereof is inhibited from eliciting a T-cell response when the complex is interrogated by one or more T-cells.

In some embodiments, the nucleic acid molecule encoding a human HLA class 1 heavy chain sequence comprises one or more mutations, wherein a cell comprising the mutated human HLA class 1 heavy chain sequence comprising the one or more mutations does not elicit an immune response when the cell is interrogated by one or more CD8 cells. In some embodiments, the mutated human HLA class 1 heavy chain sequence encodes an HLA comprising one or more mutations at one or more of amino acid residues 115, 122, 128, 194, 197, 198, 212, 214, 222, 223, 224, 225, 226, 227, 228, 229, 230, 231, 232, 233, 243, 245, 248, 262, or any combination thereof.

### Methods of Treatment

Provided herein, in another aspect, is a method of treating a disease or disorder in a subject in need thereof, comprising administering a therapeutically effective amount of the nucleic acid molecule provided herein or the immune incompetent cell provided herein.

In some embodiments, the disease is an autoimmune disease. In some embodiments, the disease is type 1 diabetes. In some embodiments, the disease is rheumatoid arthritis. In some embodiments, the disease is psoriasis. In some embodiments, the disease is psoriatic arthritis. In some embodiments, the disease is multiple sclerosis. In some embodiments, the disease is systemic lupus erythematosus. In some embodiments, the disease is inflammatory bowel disease. In some embodiments, the disease is Addison's disease. In some embodiments, the disease is Graves' disease. In some embodiments, the disease is Sjögren's syndrome. In some embodiments, the disease is Hashimoto's thyroiditis. In some embodiments, the disease is Myasthenia gravis. In some embodiments, the disease is autoimmune vasculitis. In some embodiments, the disease is pernicious anemia. In some embodiments, the disease is celiac disease. In some embodiments, the disease is vasculitis.

In some embodiments, the disease is a cancer. In some embodiments, the disease is lung cancer. In some embodiments, the disease is breast cancer. In some embodiments, the disease is colorectal cancer. In some embodiments, the disease is prostate cancer. In some embodiments, the disease is skin cancer. In some embodiments, the disease is stomach cancer. In some embodiments, the disease is leukemia. In some embodiments, the disease is lymphoma. In some embodiments, the disease is bladder cancer. In some embodiments, the disease is renal cancer. In some embodiments, the disease is endometrial cancer. In some embodiments, the disease is pancreatic cancer. In some embodiments, the disease is thyroid cancer. In some embodiments, the disease is liver cancer. In some embodiments, the disease is ovarian cancer. In some embodiments, the disease is cervical cancer.

In some embodiments, the disease is a degenerative disease. In some embodiments, the disease is Alzheimer's disease. In some embodiments, the disease is amyotrophic lateral sclerosis. In some embodiments, the disease is Friedreich's ataxia. In some embodiments, the disease is Huntington's disease. In some embodiments, the disease is Lewy body disease. In some embodiments, the disease is Parkinson's disease. In some embodiments, the disease is spinal muscular atrophy. In some embodiments, the disease is multiple sclerosis. In some embodiments, the disease is muscular dystrophy. In some embodiments, the disease is cystic fibrosis. In some embodiments, the disease is Creutzfeldt-Jakob disease. In some embodiments, the disease is Tay-Sachs disease.

In some embodiments, the administration of the immune incompetent cells provided herein treats the disease or disorder without provoking an immune response.

When one or more complexes, nucleic acid molecules, or immune incompetent cells as provided herein are administered into a human subject, the dosage will normally be determined by a physician with the dosage generally varying according to the age, weight, and response of the individual subject, as well as the severity of the subject's symptoms.

The actual dosage employed may be varied depending upon the requirements of the subject and the severity of the condition being treated. Determination of the proper dosage for a particular situation is within the skill of the art. Generally, treatment is initiated with smaller dosages which are less than the optimum dose of the one or more complexes, nucleic acid molecules, or immune incompetent cells as provided herein. Thereafter, the dosage is increased by small amounts until the optimum effect under the circumstances is reached.

The amount and frequency of administration of the one or more complexes, nucleic acid molecules, or immune incompetent cells as provided herein, and if applicable other chemotherapeutic agents and/or radiation therapy, will be regulated according to the judgment of the attending clinician (physician) considering such factors as age, condition and size of the subject as well as severity of the disease being treated.

The chemotherapeutic agent and/or radiation therapy can be administered according to therapeutic protocols well known in the art. It will be apparent to those skilled in the art that the administration of the chemotherapeutic agent and/or radiation therapy can be varied depending on the disease being treated and the known effects of the chemotherapeutic agent and/or radiation therapy on that disease. Also, in accordance with the knowledge of the skilled clinician, the therapeutic protocols (e.g., dosage amounts and times of administration) can be varied in view of the observed effects of the administered therapeutic agents (i.e., antineoplastic agent or radiation) on the subject, and in view of the observed responses of the disease to the administered therapeutic agents.

Also, in general, the one or more complexes, nucleic acid molecules, or immune incompetent cells as provided herein need not be administered in the same pharmaceutical composition as a chemotherapeutic agent, and may, because of different physical and chemical characteristics, be administered by a different route. The determination of the mode of administration and the advisability of administration, where possible, in the same pharmaceutical composition, is well within the knowledge of the skilled clinician. The initial administration can be made according to established protocols known in the art, and then, based upon the observed effects, the dosage, modes of administration and times of administration can be modified by the skilled clinician.

The particular choice of the one or more complexes, nucleic acid molecules, or immune incompetent cells as provided herein (and where appropriate, chemotherapeutic agent and/or radiation) will depend upon the diagnosis of the attending physicians and their judgment of the condition of the subject and the appropriate treatment protocol.

The one or more complexes, nucleic acid molecules, or immune incompetent cells as provided herein (and where appropriate chemotherapeutic agent and/or radiation) may be administered concurrently (e.g., simultaneously, essentially simultaneously or within the same treatment protocol) or sequentially, depending upon the nature of the proliferative disease, the condition of the subject, and the actual choice of chemotherapeutic agent and/or radiation to be administered in conjunction (i.e., within a single treatment protocol) with the one or more complexes, nucleic acid molecules, or immune incompetent cells as provided herein.

In combinational applications and uses, the one or more complexes, nucleic acid molecules, or immune incompetent cells as provided herein and the chemotherapeutic agent and/or radiation need not be administered simultaneously or essentially simultaneously, and the initial order of administration of the one or more complexes, nucleic acid molecules, or immune incompetent cells as provided herein, and the chemotherapeutic agent and/or radiation, may not be important. Thus, the one or more complexes, nucleic acid molecules, or immune incompetent cells as provided herein may be administered first followed by the administration of the chemotherapeutic agent and/or radiation; or the chemotherapeutic agent and/or radiation may be administered first followed by the administration of the one or more complexes, nucleic acid molecules, or immune incompetent cells as provided herein. This alternate administration may be repeated during a single treatment protocol. The determination of the order of administration, and the number of repetitions of administration of each therapeutic agent during a treatment protocol, is well within the knowledge of the skilled physician after evaluation of the disease being treated and the condition of the subject. For example, the chemotherapeutic agent and/or radiation may be administered first, and then the treatment continued with the administration of the one or more complexes, nucleic acid molecules, or immune incompetent cells as provided herein followed, where determined advantageous, by the administration of the chemotherapeutic agent and/or radiation, and so on until the treatment protocol is complete.

Thus, in accordance with experience and knowledge, the practicing physician can modify each protocol for the administration of one or more complexes, nucleic acid molecules, or immune incompetent cells as provided herein for treatment according to the individual subject's needs, as the treatment proceeds.

### Methods of Use

Provided herein, in another aspect, is a method of inhibiting a human leukocyte antigen (HLA) comprising contacting the HLA with a peptide that does not comprise T-cell receptor-binding residues or fragments.

In some embodiments, the peptide binds to one or more HLA binding groove domain residues of the HLA. In some embodiments, the peptide modulates a conformation of the HLA. In some embodiments, the conformation prevents a T-cell from binding the HLA.

In some embodiments, the HLA is synthetic.

### Therapeutic Efficacy

The attending clinician, in judging whether treatment is effective at the dosage administered, will consider the general well-being of the subject as well as more definite signs such as relief of disease-related symptoms, inhibition of tumor growth, actual shrinkage of the tumor, or inhibition of metastasis. Size of the tumor can be measured by standard methods such as radiological studies, e.g., CAT or MRI scan, and successive measurements can be used to judge whether or not growth of the tumor has been retarded or even reversed. Relief of disease-related symptoms such as pain, and improvement in overall condition can also be used to help judge effectiveness of treatment.

In some embodiments, therapeutic efficacy is measured based on an effect of treating a proliferative disorder, such as cancer. In general, therapeutic efficacy of the methods and compositions of the invention, with regard to the treatment of a proliferative disorder (e.g. cancer, whether benign or malignant), may be measured by the degree to which the methods and compositions promote inhibition of tumor cell proliferation, the inhibition of tumor vascularization, the eradication of tumor cells, the reduction in the rate of growth of a tumor, and/or a reduction in the size of at least one tumor. Several parameters to be considered in the determination of therapeutic efficacy are discussed herein. The proper combination of parameters for a particular situation can be established by the clinician. The progress of the inventive method in treating cancer (e.g., reducing tumor size or eradicating cancerous cells) can be ascertained using any suitable method, such as those methods currently used in the clinic to track tumor size and cancer progress. The primary efficacy parameter used to evaluate the treatment of cancer by the inventive method and compositions preferably is a reduction in the size of a tumor. Tumor size can be figured using any suitable technique, such as measurement of dimensions, or estimation of tumor volume using available computer software, such as FreeFlight software developed at Wake Forest University that enables accurate estimation of tumor volume. Tumor size can be determined by tumor visualization using, for example, CT, ultrasound, SPECT, spiral CT, MRI, photographs, and the like. In embodiments where a tumor is surgically resected after completion of the therapeutic period, the presence of tumor tissue and tumor size can be determined by gross analysis of the tissue to be resected, and/or by pathological analysis of the resected tissue.

In some desirable embodiments, the growth of a tumor is stabilized (i.e., one or more tumors do not increase more than 1%, 5%, 10%, 15%, or 20% in size, and/or do not metastasize) as a result of the inventive method and compositions. In some embodiments, a tumor is stabilized for at least about 1, 2, 3, 4, 5, 6, 7, 8, 9, 10, 11, 12, or more weeks. In some embodiments, a tumor is stabilized for at least about 1, 2, 3, 4, 5, 6, 7, 8, 9, 10, 11, 12, or more months. In some embodiments, a tumor is stabilized for at least about 1, 2, 3, 4, 5, 6, 7, 8, 9, 10, or more years. Preferably, the inventive method reduces the size of a tumor at least about 5% (e.g., at least about 10%, 15%, 20%, or 25%). More preferably, tumor size is reduced at least about 30% (e.g., at least about 35%, 40%, 45%, 50%, 55%, 60%, or 65%). Even more preferably, tumor size is reduced at least about 70% (e.g., at least about 75%, 80%, 85%, 90%, or 95%). Most preferably, the tumor is completely eliminated, or reduced below a level of detection. In some embodiments, a subject remains tumor free (e.g. in remission) for at least about 1, 2, 3, 4, 5, 6, 7, 8, 9, 10, 11, 12, or more weeks following treatment. In some embodiments, a subject remains tumor free for at least about 1, 2, 3, 4, 5, 6, 7, 8, 9, 10, 11, 12, or more months following treatment. In some embodiments, a subject remains tumor free for at least about 1, 2, 3, 4, 5, 6, 7, 8, 9, 10, or more years after treatment.

In some embodiments, the efficacy of the inventive method in reducing tumor size can be determined by measuring the percentage of necrotic (i.e., dead) tissue of a surgically resected tumor following completion of the therapeutic period. In some further embodiments, a treatment is therapeutically effective if the necrosis percentage of the resected tissue is greater than about 20% (e.g., at least about 30%, 40%, 50%, 60%, 70%, 80%, 90%, or 100%), more preferably about 90% or greater (e.g., about 90%, 95%, or 100%). Most preferably, the necrosis percentage of the resected tissue is 100%, that is, no tumor tissue is present or detectable.

The efficacy of the inventive method can be determined by a number of secondary parameters. Examples of secondary parameters include, but are not limited to, detection of new tumors, detection of tumor antigens or markers (e.g., CEA, PSA, or CA-125), biopsy, surgical downstaging (i.e., conversion of the surgical stage of a tumor from unresectable to resectable), PET scans, survival, disease progression-free survival, time to disease progression, quality of life assessments such as the Clinical Benefit Response Assessment, and the like, all of which can point to the overall progression (or regression) of cancer in a human. Biopsy is particularly useful in detecting the eradication of cancerous cells within a tissue. Radioimmunodetection (RAID) is used to locate and stage tumors using serum levels of markers (antigens) produced by and/or associated with tumors (“tumor markers” or “tumor-associated antigens”), and can be useful as a pre-treatment diagnostic predicate, a post-treatment diagnostic indicator of recurrence, and a post-treatment indicator of therapeutic efficacy. Examples of tumor markers or tumor-associated antigens that can be evaluated as indicators of therapeutic efficacy include, but are not limited to, carcinembryonic antigen (CEA), prostate-specific antigen (PSA), CA-125, CA19-9, ganglioside molecules (e.g., GM2, GD2, and GD3), MART-1, heat shock proteins (e.g., gp96), sialyl Tn (STn), tyrosinase, MUC-1, HER-2/neu, c-erb-B2, KSA, PSMA, p53, RAS, EGF-R, VEGF, MAGE, and gp100. Other tumor-associated antigens are known in the art. RAID technology in combination with endoscopic detection systems also can efficiently distinguish small tumors from surrounding tissue (see, for example, U.S. Pat. No. 4,932,412).

In additional desirable embodiments, the treatment of cancer in a human patient in accordance with the inventive method is evidenced by one or more of the following results: (a) the complete disappearance of a tumor (i.e., a complete response), (b) about a 25% to about a 50% reduction in the size of a tumor for at least four weeks after completion of the therapeutic period as compared to the size of the tumor before treatment, (c) at least about a 50% reduction in the size of a tumor for at least four weeks after completion of the therapeutic period as compared to the size of the tumor before the therapeutic period, and (d) at least a 2% decrease (e.g., about a 5%, 10%, 20%, 30%, 40%, 50%, 60%, 70%, 80% or 90% decrease) in a specific tumor-associated antigen level at about 4-12 weeks after completion of the therapeutic period as compared to the tumor-associated antigen level before the therapeutic period. While at least a 2% decrease in a tumor-associated antigen level is preferred, any decrease in the tumor-associated antigen level is evidence of treatment of a cancer in a patient by the inventive method. For example, with respect to unresectable, locally advanced pancreatic cancer, treatment can be evidenced by at least a 10% decrease in the CA19-9 tumor-associated antigen level at 4-12 weeks after completion of the therapeutic period as compared to the CA19-9 level before the therapeutic period. Similarly, with respect to locally advanced rectal cancer, treatment can be evidenced by at least a 10% decrease in the CEA tumor-associated antigen level at 4-12 weeks after completion of the therapeutic period as compared to the CEA level before the therapeutic period.

With respect to quality of life assessments, such as the Clinical Benefit Response Criteria, the therapeutic benefit of the treatment in accordance with the invention can be evidenced in terms of pain intensity, analgesic consumption, and/or the Karnofsky Performance Scale score. The treatment of cancer in a human patient alternatively, or in addition, is evidenced by (a) at least a 50% decrease (e.g., at least a 60%, 70%, 80%, 90%, or 100% decrease) in pain intensity reported by a patient, such as for any consecutive four week period in the 12 weeks after completion of treatment, as compared to the pain intensity reported by the patient before treatment, (b) at least a 50% decrease (e.g., at least a 60%, 70%, 80%, 90%, or 100% decrease) in analgesic consumption reported by a patient, such as for any consecutive four week period in the 12 weeks after completion of treatment as compared to the analgesic consumption reported by the patient before treatment, and/or (c) at least a 20 point increase (e.g., at least a 30 point, 50 point, 70 point, or 90 point increase) in the Karnofsky Performance Scale score reported by a patient, such as for any consecutive four week period in the 12 weeks after completion of the therapeutic period as compared to the Karnofsky Performance Scale score reported by the patient before the therapeutic period.

The treatment of a proliferative disorder (e.g. cancer, whether benign or malignant) in a human patient desirably is evidenced by one or more (in any combination) of the foregoing results, although alternative or additional results of the referenced tests and/or other tests can evidence treatment efficacy.

In some embodiments, tumor size is reduced as a result of the inventive method preferably without significant adverse events in the subject. Adverse events are categorized or “graded” by the Cancer Therapy Evaluation Program (CTEP) of the National Cancer Institute (NCI), with Grade 0 representing minimal adverse side effects and Grade 4 representing the most severe adverse events. Desirably, the inventive method is associated with minimal adverse events, e.g. Grade 0, Grade 1, or Grade 2 adverse events, as graded by the CTEP/NCI. However, as discussed herein, reduction of tumor size, although preferred, is not required in that the actual size of tumor may not shrink despite the eradication of tumor cells. Eradication of cancerous cells is sufficient to realize a therapeutic effect. Likewise, any reduction in tumor size is sufficient to realize a therapeutic effect.

Detection, monitoring and rating of various cancers in a human are further described in Cancer Facts and FIGS. 2001, American Cancer Society, New York, N.Y., and International Patent Application WO 01/24684. Accordingly, a clinician can use standard tests to determine the efficacy of the various embodiments of the inventive method in treating cancer. However, in addition to tumor size and spread, the clinician also may consider quality of life and survival of the patient in evaluating efficacy of treatment.

In some embodiments, administration of one or more complexes, nucleic acid molecules, or immune incompetent cells as provided herein provides improved therapeutic efficacy. Improved efficacy may be measured using any method known in the art, including but not limited to those described herein. In some embodiments, the improved therapeutic efficacy is an improvement of at least about 10%, 20%, 30%, 40%, 50%, 60%, 70%, 75%, 80%, 90%, 95%, 100%, 110%, 120%, 150%, 200%, 300%, 400%, 500%, 600%, 700%, 1000% or more, using an appropriate measure (e.g. tumor size reduction, duration of tumor size stability, duration of time free from metastatic events, duration of disease-free survival). Improved efficacy may also be expressed as fold improvement, such as at least about 2-fold, 3-fold, 4-fold, 5-fold, 6-fold, 7-fold, 8-fold, 9-fold, 10-fold, 20-fold, 30-fold, 40-fold, 50-fold, 60-fold, 70-fold, 80-fold, 90-fold, 100-fold, 1000-fold, 10000-fold or more, using an appropriate measure (e.g. tumor size reduction, duration of tumor size stability, duration of time free from metastatic events, duration of disease-free survival).

### Pharmaceutical Compositions and Formulations

The disclosure provides compositions, including pharmaceutical compositions, comprising one or more complexes, nucleic acid molecules, or immune incompetent cells as provided herein.

In some embodiments, the one or more complexes, nucleic acid molecules, or immune incompetent cells as provided herein are formulated into pharmaceutical compositions. In specific embodiments, pharmaceutical compositions are formulated in a conventional manner using one or more physiologically acceptable carriers comprising excipients and auxiliaries which facilitate processing of the one or more complexes, nucleic acid molecules, or immune incompetent cells as provided herein into preparations which can be used pharmaceutically. Proper formulation is dependent upon the route of administration chosen. Any pharmaceutically acceptable techniques, carriers, and excipients are used as suitable to formulate the pharmaceutical compositions described herein: Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pennsylvania 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999).

Provided herein are pharmaceutical compositions comprising one or more complexes, nucleic acid molecules, or immune incompetent cells as provided herein and a pharmaceutically acceptable diluent(s), excipient(s), or carrier(s). In certain embodiments, the one or more complexes, nucleic acid molecules, or immune incompetent cells as provided herein are administered as pharmaceutical compositions in which the one or more complexes, nucleic acid molecules, or immune incompetent cells as provided herein are mixed with other active ingredients, as in combination therapy. In specific embodiments, the pharmaceutical compositions include one or more complexes, nucleic acid molecules, or immune incompetent cells as provided herein.

A pharmaceutical composition, as used herein, refers to a mixture of one or more complexes, nucleic acid molecules, or immune incompetent cells as provided herein with other chemical components, such as carriers, stabilizers, diluents, dispersing agents, suspending agents, thickening agents, and/or excipients. In certain embodiments, the pharmaceutical composition facilitates administration of the one or more complexes, nucleic acid molecules, or immune incompetent cells as provided herein to an organism. In some embodiments, in practicing the methods of treatment or use provided herein, therapeutically effective amounts of one or more complexes, nucleic acid molecules, or immune incompetent cells as provided herein are administered in a pharmaceutical composition to a mammal having a disease or condition to be treated. In specific embodiments, the mammal is a human. In certain embodiments, therapeutically effective amounts vary depending on the severity of the disease, the age and relative health of the subject and other factors. The one or more complexes, nucleic acid molecules, or immune incompetent cells as provided herein are used singly or in combination with one or more therapeutic agents as components of mixtures.

In one embodiment, one or more complexes, nucleic acid molecules, or immune incompetent cells as provided herein are formulated in an aqueous solution. In specific embodiments, the aqueous solution is selected from, by way of example only, a physiologically compatible buffer, such as Hank's solution, Ringer's solution, or physiological saline buffer. In other embodiments, one or more complexes, nucleic acid molecules, or immune incompetent cells as provided herein are formulated for transmucosal administration. In specific embodiments, transmucosal formulations include penetrants that are appropriate to the barrier to be permeated. In still other embodiments wherein the one or more complexes, nucleic acid molecules, or immune incompetent cells as provided herein are formulated for other parenteral injections, appropriate formulations include aqueous or nonaqueous solutions. In specific embodiments, such solutions include physiologically compatible buffers and/or excipients.

In still other embodiments, the one or more complexes, nucleic acid molecules, or immune incompetent cells as provided herein are formulated for parental injection, including formulations suitable for bolus injection or continuous infusion. In specific embodiments, formulations for injection are presented in unit dosage form (e.g., in ampoules) or in multi-dose containers. Preservatives are, optionally, added to the injection formulations. In still other embodiments, the pharmaceutical composition of one or more complexes, nucleic acid molecules, or immune incompetent cells as provided herein is formulated in a form suitable for parenteral injection as sterile suspension, solution or emulsion in oily or aqueous vehicles. Parenteral injection formulations optionally contain formulatory agents such as suspending, stabilizing and/or dispersing agents. In specific embodiments, pharmaceutical formulations for parenteral administration include aqueous solutions of the one or more complexes, nucleic acid molecules, or immune incompetent cells as provided herein in water-soluble form. In additional embodiments, suspensions of the one or more complexes, nucleic acid molecules, or immune incompetent cells as provided herein are prepared as appropriate oily injection suspensions. Suitable lipophilic solvents or vehicles for use in the pharmaceutical compositions described herein include, by way of example only, fatty oils such as sesame oil, or synthetic fatty acid esters, such as ethyl oleate or triglycerides, or liposomes. In certain specific embodiments, aqueous injection suspensions contain substances which increase the viscosity of the suspension, such as sodium carboxymethyl cellulose, sorbitol, or dextran. Optionally, the suspension contains suitable stabilizers or agents which increase the solubility of the one or more complexes, nucleic acid molecules, or immune incompetent cells as provided herein to allow for the preparation of highly concentrated solutions. Alternatively, in other embodiments, the active ingredient is in powder form for constitution with a suitable vehicle, e.g., sterile pyrogen-free water, before use.

In certain embodiments, pharmaceutical compositions are formulated in any conventional manner using one or more physiologically acceptable carriers comprising excipients and auxiliaries which facilitate processing of the one or more complexes, nucleic acid molecules, or immune incompetent cells as provided herein into preparations which can be used pharmaceutically. Proper formulation is dependent upon the route of administration chosen. Any pharmaceutically acceptable techniques, carriers, and excipients are optionally used as suitable. Pharmaceutical compositions comprising the one or more complexes, nucleic acid molecules, or immune incompetent cells as provided herein are manufactured in a conventional manner, such as, by way of example only, by means of conventional mixing, dissolving, granulating, dragee-making, levigating, emulsifying, encapsulating, entrapping or compression processes.

In some embodiments, a pharmaceutical composition comprising one or more complexes, nucleic acid molecules, or immune incompetent cells as provided herein illustratively takes the form of a liquid where the agents are present in solution, in suspension or both. Typically when the composition is administered as a solution or suspension a first portion of the agent is present in solution and a second portion of the agent is present in particulate form, in suspension in a liquid matrix. In some embodiments, a liquid composition includes a gel formulation. In other embodiments, the liquid composition is aqueous.

In certain embodiments, a useful aqueous suspension contains one or more polymers as suspending agents. Useful polymers include water-soluble polymers such as cellulosic polymers, e.g., hydroxypropyl methylcellulose, and water-insoluble polymers such as cross-linked carboxyl-containing polymers. Certain pharmaceutical compositions described herein comprise a mucoadhesive polymer, selected for example from carboxymethylcellulose, carbomer (acrylic acid polymer), poly(methylmethacrylate), polyacrylamide, polycarbophil, acrylic acid/butyl acrylate copolymer, sodium alginate and dextran.

Useful pharmaceutical compositions also, optionally, include solubilizing agents to aid in the solubility of one or more complexes, nucleic acid molecules, or immune incompetent cells as provided herein. The term “solubilizing agent” generally includes agents that result in formation of a micellar solution or a true solution of the agent. Certain acceptable nonionic surfactants, for example polysorbate 80, are useful as solubilizing agents, as can ophthalmically acceptable glycols, polyglycols, e.g., polyethylene glycol 400, and glycol ethers.

Furthermore, useful pharmaceutical compositions optionally include one or more pH adjusting agents or buffering agents, including acids such as acetic, boric, citric, lactic, phosphoric and hydrochloric acids; bases such as sodium hydroxide, sodium phosphate, sodium borate, sodium citrate, sodium acetate, sodium lactate and tris-hydroxymethylaminomethane; and buffers such as citrate/dextrose, sodium bicarbonate and ammonium chloride. Such acids, bases and buffers are included in an amount required to maintain pH of the composition in an acceptable range.

Additionally, useful compositions also, optionally, include one or more salts in an amount required to bring osmolality of the composition into an acceptable range. Such salts include those having sodium, potassium or ammonium cations and chloride, citrate, ascorbate, borate, phosphate, bicarbonate, sulfate, thiosulfate or bisulfite anions; suitable salts include sodium chloride, potassium chloride, sodium thiosulfate, sodium bisulfite and ammonium sulfate.

Other useful pharmaceutical compositions optionally include one or more preservatives to inhibit microbial activity. Suitable preservatives include mercury-containing substances such as merfen and thiomersal; stabilized chlorine dioxide; and quaternary ammonium compounds such as benzalkonium chloride, cetyltrimethylammonium bromide and cetylpyridinium chloride.

Still other useful compositions include one or more surfactants to enhance physical stability or for other purposes. Suitable nonionic surfactants include polyoxyethylene fatty acid glycerides and vegetable oils, e.g., polyoxyethylene (60) hydrogenated castor oil; and polyoxyethylene alkylethers and alkylphenyl ethers, e.g., octoxynol 10, octoxynol 40.

Still other useful compositions include one or more antioxidants to enhance chemical stability where required. Suitable antioxidants include, by way of example only, ascorbic acid and sodium metabisulfite.

In certain embodiments, aqueous suspension compositions are packaged in single-dose non-reclosable containers. Alternatively, multiple-dose reclosable containers are used, in which case it is typical to include a preservative in the composition.

In alternative embodiments, other delivery systems are employed. Liposomes and emulsions are examples of delivery vehicles or carriers useful herein. In additional embodiments, the one or more complexes, nucleic acid molecules, or immune incompetent cells as provided herein are delivered using a sustained-release system, such as semipermeable matrices of solid hydrophobic polymers containing the therapeutic agent. Various sustained-release materials are useful herein. Depending on the chemical nature and the biological stability of the therapeutic reagent, additional strategies for protein stabilization are employed.

In certain embodiments, the formulations described herein comprise one or more antioxidants, metal chelating agents, thiol containing compounds and/or other general stabilizing agents. Examples of such stabilizing agents, include, but are not limited to: (a) about 0.5% to about 2% w/v glycerol, (b) about 0.1% to about 1% w/v methionine, (c) about 0.1% to about 2% w/v monothioglycerol, (d) about 1 mM to about 10 mM EDTA, (e) about 0.01% to about 2% w/v ascorbic acid, (0.003% to about 0.02% w/v polysorbate 80, (g) 0.001% to about 0.05% w/v. polysorbate 20, (h) arginine, (i) heparin, (j) dextran sulfate, (k) cyclodextrins, (1) pentosan polysulfate and other heparinoids, (m) divalent cations such as magnesium and zinc; or (n) combinations thereof.

### Routes of Administration

Suitable routes of administration include, but are not limited to, oral, intravenous, rectal, aerosol, parenteral, ophthalmic, pulmonary, transmucosal, transdermal, vaginal, otic, nasal, and topical administration. In addition, by way of example only, parenteral delivery includes intramuscular, subcutaneous, intravenous, intramedullary injections, as well as intrathecal, direct intraventricular, intraperitoneal, intralymphatic, and intranasal injections.

In certain embodiments, a composition comprising immune incompetent cells as provided herein is administered in a local rather than systemic manner, for example, via injection of the composition directly into an organ, often in a depot preparation or sustained release formulation. In specific embodiments, long acting formulations are administered by implantation (for example subcutaneously or intramuscularly) or by intramuscular injection. Furthermore, in other embodiments, the composition is delivered in a targeted drug delivery system, for example, in a liposome coated with an organ-specific antibody. In such embodiments, the liposomes are targeted to and taken up selectively by the organ. In yet other embodiments, a composition comprising immune incompetent cells as provided herein is provided in the form of a rapid release formulation, in the form of an extended release formulation, or in the form of an intermediate release formulation.

### Kits and Articles of Manufacture

For use in the therapeutic applications described herein, kits and articles of manufacture are also provided. In some embodiments, such kits comprise a carrier, package, or container that is compartmentalized to receive one or more containers such as vials, tubes, and the like, each of the container(s) comprising one of the separate elements to be used in a method described herein. Suitable containers include, for example, bottles, vials, syringes, and test tubes. The containers are formed from a variety of materials such as glass or plastic.

The articles of manufacture provided herein contain packaging materials. Packaging materials for use in packaging pharmaceutical products Include those found in, e.g., U.S. Pat. Nos. 5,323,907, 5,052,558 and 5,033,252. Examples of pharmaceutical packaging materials include, but are not limited to, blister packs, bottles, tubes, inhalers, pumps, bags, vials, containers, syringes, bottles, and any packaging material suitable for a selected formulation and intended mode of administration and treatment. For example, the container(s) includes one or more nucleic acid molecules or immune incompetent cells described herein, optionally in a composition. The container(s) optionally have a sterile access port (for example the container is an intravenous solution bag or a vial having a stopper pierceable by a hypodermic injection needle). Such kits optionally comprise a composition with an identifying description or label or instructions relating to its use in the methods described herein.

For example, a kit typically includes one or more additional containers, each with one or more of various materials (such as reagents, optionally in concentrated form, and/or devices) desirable from a commercial and user standpoint for use of a composition described herein. Non-limiting examples of such materials include, but not limited to, buffers, diluents, filters, needles, syringes; carrier, package, container, vial and/or tube labels listing contents and/or instructions for use, and package inserts with instructions for use. A set of instructions will also typically be included. A label is optionally on or associated with the container. For example, a label is on a container when letters, numbers or other characters forming the label are attached, molded or etched into the container itself, a label is associated with a container when it is present within a receptacle or carrier that also holds the container, e.g., as a package insert. In addition, a label is used to indicate that the contents are to be used for a specific therapeutic application. In addition, the label indicates directions for use of the contents, such as in the methods described herein. In certain embodiments, the pharmaceutical composition is presented in a pack or dispenser device which contains one or more unit dosage forms comprising one or more complexes, nucleic acid molecules, immune incompetent cells, or pharmaceutical compositions thereof provided herein. The pack for example contains metal or plastic foil, such as a blister pack. Or, the pack or dispenser device is accompanied by instructions for administration. Or, the pack or dispenser is accompanied with a notice associated with the container in form prescribed by a governmental agency regulating the manufacture, use, or sale of pharmaceuticals, which notice is reflective of approval by the agency of the form of the drug for human or veterinary administration. Such notice, for example, is the labeling approved by the U.S. Food and Drug Administration for prescription drugs, or the approved product insert. In some embodiments, compositions comprising immune incompetent cells formulated in a compatible pharmaceutical carrier are prepared, placed in an appropriate container, and labeled for treatment of an indicated condition.

### EXAMPLES

### Example 1: Identification of Anchor Amino Acids of Peptide

Most HLA Class 1 alleles preferentially bind 9- to 12-mer peptides, and the majority of alleles accommodate peptides with anchor residues at the second and last positions, as these residues are buried in the peptide binding groove.

An analysis of 9-mer peptides was performed to determine the peptide binding preferences for all alleles of HLA-A, HLA-B, and HLA-C. The amino acid diversity was determined for the anchor residues at the second (e.g. P2) and last (e.g. P9 for a 9-mer) positions. Longer peptides also bind at these anchor positions, with the extra length accommodated by either bulging in the middle or overhanging outside of the peptide binding groove.

The HLA-A alleles investigated included HLA-A*01:01, HLA-A*02:01, HLA-A*02:02, HLA-A*02:03, HLA-A*02:04, HLA-A*02:05, HLA-A*02:06, HLA-A*02:07, HLA-A*02:11, HLA-A*03:01, HLA-A*11:01, HLA-A*11:02, HLA-A*23:01, HLA-A*24:02, HLA-A*24:07, HLA-A*25:01, HLA-A*26:01, HLA-A*29:02, HLA-A*30:01, HLA-A*30:02, HLA-A*31:01, HLA-A*32:01, HLA-A*33:01, HLA-A*33:03, HLA-A*34:01, HLA-A*34:02, HLA-A*36:01, HLA-A*66:01, HLA-A*68:01, HLA-A*68:02, and HLA-A*74:01.

The conserved anchor residues of each investigated HLA-A alleles are summarized in Table 1:

TABLE 1
Conserved Anchor Residues of Each HLA-A Allele

| | Second Amino Acid | Last Amino Acid |
|---|---|---|
| HLA Allele | Anchor Residue | Anchor Residue |
| HLA-A*01 | S/T | Y |
| HLA-A*02 | L/I/V/M | I/V/L |
| HLA-A*03 | L/V/I/T | K/Y |
| HLA-A*11 | T/V | K |
| HLA-A*23 | Y/F | F/L |
| HLA-A*24 | Y/F | F/L |
| HLA-A*25 | V/T | Y/W/F |
| HLA-A*26 | V/T | Y/F |
| HLA-A*29 | F/V | Y |
| HLA-A*30 | V/T | K/Y |
| HLA-A*31 | V/T/Y | R |
| HLA-A*32 | L/T/V | W/F/Y |
| HLA-A*33 | Y/V | R |
| HLA-A*34 | V/T | R/K |
| HLA-A*36 | T/S | Y |
| HLA-A*66 | V/T | R |
| HLA-A*68 | V/T | R/V |
| HLA-A*69 (n = 20) | V/S/H | Y |
| HLA-A*74 | L/V/T | R |

The frequency of second amino acid anchor residues amongst the investigated HLA-A alleles is summarized in Table 2:

TABLE 2
Frequency of Second Amino Acid Anchor Residues of HLA-A

| Second Amino | | | |
|---|---|---|---|
| Acid Anchor | | Alleles Covered | |
| Residue | HLA Alleles | (N = 19) | |
| S | HLA-A*01, HLA-A*36, HLA-A*69 | 3 | (16%) |
| T | HLA-A*01, HLA-A*03, HLA- | 13 | (68%) |
| | A*11, HLA-A*25, HLA-A*26, | | |
| | HLA-A*30, HLA-A*31, HLA- | | |
| | A*32, HLA-A*34, HLA-A*36, | | |
| | HLA-A*66, HLA-A*68, HLA-A*74 | | |
| V | HLA-A*02, HLA-A*03, HLA- | 15 | (79%) |
| | A*11, HLA-A*25, HLA- | | |
| | A*26,HLA-A*29, HLA-A*30, | | |
| | HLA-A*31, HLA-A*32, HLA- | | |
| | A*33, HLA-A*34, HLA-A*66, | | |
| | HLA-A*68, HLA-A*69, HLA-A*74 | | |
| I | HLA-A*02, HLA-A*03 | 2 | (11%) |
| L | HLA-A*02, HLA-A*03, HLA- | 4 | (21%) |
| | A*32, HLA-A*74 | | |
| M | HLA-A*02 | 1 | (5%) |
| F | HLA-A*23, HLA-A*24, HLA-A*29 | 3 | (16%) |
| Y | HLA-A*23, HLA-A*24, HLA- | 4 | (21%) |
| | A*31, HLA-A*33 | | |

The frequency of last amino acid anchor residues amongst the investigated HLA-A alleles is summarized in Table 3:

TABLE 3
Frequency of Last Amino Acid Anchor Residues of HLA-A

| Last Amino | | |
|---|---|---|
| Acid Anchor | | Alleles Covered |
| Residue | HLA Alleles | (N = 19) |
| I | HLA-A*02 | 1 (5%) |
| V | HLA-A*02, HLA-A*68 | 2 (11%) |
| L | HLA-A*02, HLA-A*23, HLA-A*24 | 3 (16%) |
| F | HLA-A*23, HLA-A*24, HLA- | 5 (26%) |
| | A*25, HLA-A*26, HLA-A*32 | |
| Y | HLA-A*01, HLA-A*03, HLA- | 9 (47%) |
| | A*25, HLA-A*26, HLA-A*29, | |
| | HLA-A*30, HLA-A*32, HLA- | |
| | A*36, HLA-A*69 | |
| R | HLA-A*31, HLA-A*33, HLA- | 6 (32%) |
| | A*34, HLA-A*66, HLA-A*68, | |
| | HLA-A*74 | |
| K | HLA-A*03, HLA-A*11, HLA- | 4 (21%) |
| | A*30, HLA-A*34 | |
| W | HLA-A*25, HLA-A*32 | 2 (11%) |

The HLA-B alleles investigated included HLA-B*07:02, HLA-B*07:04, HLA-B*08:01, HLA-B*13:01, HLA-B*13:02, HLA-B*14:02, HLA-B*15:01, HLA-B*15:02, HLA-B*15:03, HLA-B*15:10, HLA-B*15:17, HLA-B*18:01, HLA-B*27:05, HLA-B*35:01, HLA-B*35:03, HLA-B*35:07, HLA-B*37:01, HLA-B*38:01, HLA-B*38:02, HLA-B*40:01, HLA-B*40:02, HLA-B*40:06, HLA-B*42:01, HLA-B*44:02, HLA-B*44:03, HLA-B*45:01, HLA-B*46:01, HLA-B*49:01, HLA-B*50:01, HLA-B*51:01, HLA-B*52:01, HLA-B*53:01, HLA-B*54:01, HLA-B*55:01, HLA-B*55:02, HLA-B*56:01, HLA-B*57:01, HLA-B*57:03, HLA-B*58:01, and HLA-B*58:02.

The conserved anchor residues of each investigated HLA-B alleles are summarized in Table 4:

TABLE 4
Conserved Anchor Residues of Each HLA-B Allele

| | Second Amino Acid | Last Amino Acid |
|---|---|---|
| HLA Allele | Anchor Residue | Anchor Residue |
| HLA-B*07 | P | L |
| HLA-B*08 | L/P | L/V/F |
| HLA-B*13 | Q/L | L/I/F |
| HLA-B*14 | R | L/V |
| HLA-B*15 | Q/L/V/K/H/S/T | Y/F/L/V/W |
| HLA-B*18 | E | Y/F |
| HLA-B*27 | R | L/F/RY |
| HLA-B*35 | P/A | Y/F/L/M |
| HLA-B*37 | D/E | F/L/M/V |
| HLA-B*38 | H | L/I/F |
| HLA-B*40 | E | L/I/F/V/A/P |
| HLA-B*42 | P | L/F/V |
| HLA-B*44 | E | F/W/Y/L |
| HLA-B*45 | E | A/P/V |
| HLA-B*46 | A/L | Y/F/L |
| HLA-B*49 | E | V/I/L |
| HLA-B*50 | E | A/P/V |
| HLA-B*51 | P/A | I/V |
| HLA-B*52 | Q/M | I/V |
| HLA-B*53 | P/A | W/F/L |
| HLA-B*54 | P/A | I/A/V |
| HLA-B*55 | P | A/V |
| HLA-B*56 | P | A/V |
| HLA-B*57 | S/T/A | W/F/Y/L |
| HLA-B*58 | S/T/A | W/F/Y/L/I |

The frequency of second amino acid anchor residues amongst the investigated HLA-B alleles is summarized in Table 5:

TABLE 5
Frequency of Second Amino Acid Anchor Residues of HLA-B

| Second Amino | | | |
|---|---|---|---|
| Acid Anchor | | Alleles Covered | |
| Residue | HLA Alleles | (N = 25) | |
| A | HLA-B*35, HLA-B*46, HLA- | 7 | (28%) |
| | B*51, HLA-B*53, HLA-B*54, | | |
| | HLA-B*57, HLA-B*58 | | |
| S | HLA-B*15, HLA-B*57, HLA-B*58 | 3 | (12%) |
| T | HLA-B*15, HLA-B*57, HLA-B*58 | 3 | (12%) |
| V | HLA-B*15 | 1 | (4%) |
| L | HLA-B*08, HLA-B*13, HLA- | 4 | (16%) |
| | B*15, HLA-B*46 | | |
| M | HLA-B*52 | 1 | (4%) |
| H | HLA-B*15, HLA-B*38 | 2 | (8%) |
| D | HLA-B*37 | 1 | (4%) |
| E | HLA-B*18, HLA-B*37, HLA- | 7 | (28%) |
| | B*40, HLA-B*44, HLA-B*45, | | |
| | HLA-B*49, HLA-B*50 | | |
| Q | HLA-B*13, HLA-B*15, HLA-B*52 | 3 | (12%) |
| R | HLA-B*14, HLA-B*27 | 2 | (8%) |
| K | HLA-B*15 | 1 | (4%) |
| P | HLA-B*07, HLA-B*08, HLA- | 9 | (36%) |
| | B*35, HLA-B*42, HLA-B*51, | | |
| | HLA-B*53, HLA-B*54, HLA- | | |
| | B*55, HLA-B*56 | | |

The frequency of last amino acid anchor residues amongst the investigated HLA-B alleles is summarized in Table 6:

TABLE 6
Frequency of Last Amino Acid Anchor Residues of HLA-B

| Last Amino | | | |
|---|---|---|---|
| Acid Anchor | | Alleles Covered | |
| Residue | HLA Alleles | (N = 25) | |
| A | HLA-B*40, HLA-B*45, HLA- | 6 | (24%) |
| | B*50, HLA-B*54, HLA-B*55, | | |
| | HLA-B*56 | | |
| V | HLA-B*08, HLA-B*14, HLA- | 14 | (56%) |
| | B*15, HLA-B*37, HLA-B*40, | | |
| | HLA-B*42, HLA-B*45, HLA- | | |
| | B*49, HLA-B*50, HLA-B*51, | | |
| | HLA-B*52, HLA-B*54, HLA- | | |
| | B*55, HLA-B*56 | | |
| I | HLA-B*13, HLA-B*38, HLA- | 8 | (32%) |
| | B*40, HLA-B*49, HLA-B*51, | | |
| | HLA-B*52, HLA-B*54, HLA-B*58 | | |
| L | HLA-B*07, HLA-B*08, HLA- | 17 | (68%) |
| | B*13, HLA-B*14, HLA-B*15, | | |
| | HLA-B*27, HLA-B*35, HLA- | | |
| | B*37, HLA-B*38, HLA-B*40, | | |
| | HLA-B*42, HLA-B*44, HLA- | | |
| | B*46, HLA-B*49, HLA-B*53, | | |
| | HLA-B*57, HLA-B*58 | | |
| F | HLA-B*08, HLA-B*13, HLA- | 15 | (60%) |
| | B*15, HLA-B*18, HLA-B*27, | | |
| | HLA-B*35, HLA-B*37, HLA- | | |
| | B*38, HLA-B*40, HLA-B*42, | | |
| | HLA-B*44, HLA-B*46, HLA- | | |
| | B*53, HLA-B*57, HLA-B*58 | | |
| Y | HLA-B*15, HLA-B*18, HLA- | 8 | (32%) |
| | B*27, HLA-B*35, HLA-B*44, | | |
| | HLA-B*46, HLA-B*57, HLA-B*58 | | |
| M | HLA-B*35, HLA-B*37, HLA-B*44 | 3 | (12%) |
| W | HLA-B*15, HLA-B*53, HLA- | 4 | (16%) |
| | B*57, HLA-B*58 | | |
| P | HLA-B*40, HLA-B*45, HLA-B*50 | 3 | (12%) |
| R | HLA-B*27 | 1 | (4%) |

The HLA-C alleles investigated included HLA-C*01:02, HLA-C*02:02, HLA-C*03:02, HLA-C*03:03, HLA-C*03:04, HLA-C*04:01, HLA-C*04:03, HLA-C*05:01, HLA-C*06:02, HLA-C*07:01, HLA-C*07:02, HLA-C*07:04, HLA-C*08:01, HLA-C*08:02, HLA-C*12:02, HLA-C*12:03, HLA-C*14:02, HLA-C*14:03, HLA-C*15:02, HLA-C*16:01, and HLA-C*17:01.

The conserved anchor residues of each investigated HLA-C alleles are summarized in Table 7:

TABLE 7
Conserved Anchor Residues of Each HLA-C Allele

| | Second Amino Acid | Last Amino Acid |
|---|---|---|
| HLA Allele | Anchor Residue | Anchor Residue |
| HLA-C*01 | A/L/S | L/V/M |
| HLA-C*02 | A/S/T/Q/V | F/Y/L/V |
| HLA-C*03 | A/S/I | L/F/Y/M |
| HLA-C*04 | F/Y/W/A/T/V | L/F/V/M/I |
| HLA-C*05 | A/L/S/V | L/F/V/M |
| HLA-C*06 | R/A/Y | L/V/I |
| HLA-C*07 | R/T/N/Y | L/Y/F/M |
| HLA-C*08 | A/S/V | L/V |
| HLA-C*12 | A/I/S | F/Y/L/V |
| HLA-C*14 | Y/F | L/F/Y |
| HLA-C*15 | A/T/A | V/L |
| HLA-C*16 | A/S/T | L/Y/F |
| HLA-C*17 | A | L |

The frequency of second amino acid anchor residues amongst the investigated HLA-C alleles is summarized in Table 8:

TABLE 8
Frequency of Second Amino Acid Anchor Residues of HLA-C

| Second Amino | | | |
|---|---|---|---|
| Acid Anchor | | Alleles Covered | |
| Residue | HLA Alleles | (N = 13) | |
| A | HLA-C*01, HLA-C*02, HLA- | 11 | (85%) |
| | C*03, HLA-C*04, HLA-C*05, | | |
| | HLA-C*06, HLA-C*08, HLA-C*12, | | |
| | HLA-C*15, HLA-C*16, HLA- | | |
| | C*17 | | |
| S | HLA-C*01, HLA-C*02, HLA- | 7 | (54%) |
| | C*03, HLA-C*05, HLA-C*08, | | |
| | HLA-C*12, HLA-C*16 | | |
| T | HLA-C*02, HLA-C*04, HLA- | 5 | (38%) |
| | C*07, HLA-C*15, HLA-C*16 | | |
| V | HLA-C*02, HLA-C*04, HLA- | 4 | (31%) |
| | C*05, HLA-C*08 | | |
| I | HLA-C*03, HLA-C*12 | 2 | (15%) |
| L | HLA-C*01, HLA-C*05 | 2 | (15%) |
| F | HLA-C*04, HLA-C*14 | 2 | (15%) |
| Y | HLA-C*04, HLA-C*06, HLA- | 4 | (31%) |
| | C*07, HLA-C*14 | | |
| Q | HLA-C*02 | 1 | (8%) |
| R | HLA-C*06, HLA-C*07 | 2 | (15%) |
| N | HLA-C*07 | 1 | (8%) |
| W | HLA-C*04 | 1 | (8%) |

The frequency of last amino acid anchor residues amongst the investigated HLA-C alleles is summarized in Table 9:

TABLE 9
Frequency of Last Amino Acid Anchor Residues of HLA-C

| Last Amino | | |
|---|---|---|
| Acid Anchor | | Alleles Covered |
| Residue | HLA Alleles | (N = 13) |
| L | HLA-C*01, HLA-C*02, HLA- | 13 (100%) |
| | C*03, HLA-C*04, HLA-C*05, | |
| | HLA-C*06, HLA-C*07, HLA- | |
| | C*08, HLA-C*12, HLA-C*14, | |
| | HLA-C*15, HLA-C*16, HLA-C*17 | |
| V | HLA-C*01, HLA-C*02, HLA- | 8 (61%) |
| | C*04, HLA-C*05, HLA-C*06, | |
| | HLA-C*08, HLA-C*12, HLA-C*15 | |
| M | HLA-C*01, HLA-C*03, HLA- | 5 (38%) |
| | C*04, HLA-C*05, HLA-C*07 | |
| F | HLA-C*02, HLA-C*03, HLA- | 8 (61%) |
| | C*04, HLA-C*05, HLA-C*07, | |
| | HLA-C*12, HLA-C*14, HLA-C*16 | |
| Y | HLA-C*02, HLA-C*03, HLA- | 6 (46%) |
| | C*07, HLA-C*12, HLA-C*14, | |
| | HLA-C*16 | |
| I | HLA-C*04, HLA-C*06 | 2 (15%) |

### Example 2: Preparation of Nucleic Acid Molecule

A nucleic acid molecule comprising the synthetic HLA sequence is prepared according to known methods in the art. For example, the nucleic acid molecule is prepared by solid-phase oligonucleotide synthesis using nucleoside phosphoramidites. Alternatively, portions of the nucleic acid molecule are prepared by solid-phase oligonucleotide synthesis using nucleoside phosphoramidites, and the portions are assembled into the complete nucleic acid molecule according to known methods in the art. For example, the portions are assembled using endonuclease-mediated assembly, site-specific recombination, or long-overlap based assembly.

### Example 3: Preparation of Complex

A recombinant plasmid comprising the nucleic acid molecule comprising the synthetic HLA sequence is prepared according to known procedures for the preparation of recombinant plasmids. For example, the plasmid is cut with a restriction enzyme and the nucleic acid molecule comprising the synthetic HLA sequence is introduced into the plasmid via the use of DNA ligase.

The recombinant plasmid is subsequently transformed into a population of cells. The successfully transformed cells are selected according to known methods in the art, such as with the use of a selection antibiotic. The selected cells are cultured and induced to produce the complex. The cells are subsequently lysed and the complex is purified according to protein purification techniques known in the art, such as size exclusion chromatography, hydrophobic interaction chromatography, ion exchange chromatography, free-flow electrophoresis, immunoaffinity chromatography, immunoprecipitation, or high performance liquid chromatography.

Alternatively, the complex is preparing by solid phase peptide synthesis according to known methods in the art.

### Example 4: Preparation of Immune Incompetent Cells

An immune incompetent cell, such as an immune incompetent stem cell, is prepared by introducing the nucleic acid molecule of Example 2 into the cell's genome according to methods known in the art. For example, the nucleic acid molecule is delivered to the cell via a viral vector. Alternatively, the nucleic acid molecule is delivered to the cell via a non-viral method, such as the use of naked DNA injection, electroporation, gene gun, sonoporation, magnetofection, lipoplexes, dendrimers, inorganic nanoparticles, CRISPR, mRNA, or siRNA.

Alternatively, an immune incompetent cell, such as an immune incompetent stem cell, is prepared by incubating the cell with the complex of Example 3.

### Example 5: Immune Cell Proliferation Assay

Immune cells, for example T cells, are cultured and incubated with the immune incompetent cell of Example 4 and a radio-labelled nucleotide, such as ³H-thymidine. Following incubation, the immune cells are centrifugated and washed and the radioactivity is measured and compared to a control group of cells not treated with the immune incompetent cell of Example 4.

### Example 6: Treatment of Disease or Disorder

The immune incompetent cells of Example 4 are administered to a patient suffering from a disease or disorder. The patient's condition is monitored according to therapeutic standards of care according to the disease or disorder.

### Example 7: Transgenic Cloning of synHLA Constructs into B2Mnull EBV and K562 Cells

Generation of β2M^{-/-} EBV-transformed B-cell lines

CRISPR/Cas9 is used to mutate (32M in 2 EBV lines ((9031 and JK). EBV cells are transfected with Cas9 complex and HLA class I low/negative EBV cells are sorted. The cells are grown, and the absence of surface HLA class I is confirmed.

Generation of NK cell lines and testing K562 and/or (32M EBV-transformed B-cell lines for susceptibility to NK-cell mediated killing

NK cells (CD3⁻, CD56⁺) are sorted and grown in the presence of IL-2, -15 and IL-21. NK cells from PBMC are sorted and expanded in cytokines (IL-2 and IL-15). The purity of NK cell lines may be confirmed by staining with CD3, CD4, CD8, CD56. The killing of K562 and/or WT and β2M/HLA class I^{-/-} EBV cells is compared.

Express synHLA Protein(s) in K562 and/or EBV-Transformed B-Cell Lines

A synHLA gene encoding a synHLA construct as described herein is cloned into either a lentiviral vector (pRRLSIN) or an EBV episomal vector (pCE). A gene block is constructed that encodes the synHLA codon-optimized for expression in mammalian cells. This gene may be cloned into pRRLSIN or pCE using, e.g., Infusion cloning. The construct may be sequenced to confirm that the sequence is correct and a plasmid is prepared. pRRLSIN may be used to make lentiviruses which are used to transduce K562 cells. pCE is electroporated in EBV cells and transfected cells selected by antibiotic selection. Transduced K562 are stained for synHLA construct and selected by FACS sorting. Transfected EBV cells (WT and (32M/HLA class I^{-/-}) are selected in the presence of antibody and the expression of the synHLA construct(s) is assessed by flow cytometry with a monoclonal antibody. K562 or EBV cells expressing the synHLA construct(s) are tested for recognition and killing by NK cell lines generated as described above.

Determine if EBV Cells, or K562 Cells Expressing synHLA can Activate Antigen Specific CD8⁺ T-cell clones.

Using either EBV-transformed B-cell lines: with or without HLA class I; and with or without synHLA, or K562 transduced with the appropriate HLA class I construct, the following is tested: Response of influenza MP58-66 epitope specific CD8⁺ T-cell clones, described above, pulsed with the cognate peptide. Proliferation of allogeneic primary CFSE-labelled CD8⁺ T cells, purified by magnetic beads or flow cytometry, in response to K562 and/or EBV-transformed B cell lines described above, using the method of Mannering, S. I. et al. A sensitive method for detecting proliferation of rare autoantigen-specific human T cells. J Immunol Methods, 2003, 283, 173-183. T-cell killing of transduced cells will also be assessed by measuring interferon gamma secretion.

### Example 8: Recombinant Bacterial Expression of HLA Complexes

HLA complexes, including synthetic HLA complexes (synHLA) as described herein (e.g., those listed in Table 10) are recombinantly expressed in bacteria. Briefly, bacterial cells are transfected with nucleic acids encoding the sequence of a synHLA construct as described herein. The protein may be isolated and purified from the bacteria. If the protein is present as inclusion bodies, the protein may be refolded (e.g., by denaturing with a chaotropic agent and transferring to a dilute aqueous environment). Following the refolding step, the refolded protein may be purified (e.g., by immobilized metal-affinity chromatography). Isolated protein (e.g., 1 μg) may be subjected to SDS-PAGE in the presence or absence of a reducing agent (e.g., dithiothreitol; DTT) and visualized (e.g., using Coomassie Blue staining).

TABLE 10
HLA complex sequences

| | SEQ | |
|---|---|---|
| | ID | |
| Description | NO: | Sequence |
| Single chain | 1 | GILGFVFTLGGGGSGGGGSGGGGSI |
| trimer | | QRTPKIQVYSRHPAENGKSNFLNCY |
| | | VSGFHPSDIEVDLLKNGERIEKVEH |
| | | SDLSFSKDWSFYLLYYTEFTPTEKD |
| | | EYACRVNHVTLSQPKIVKWDRDMGG |
| | | GGSGGGGSGGGGSGSHSMRYFFTSV |
| | | SRPGRGEPRFIAVGYVDDTQFVRFD |
| | | SDAASQRMEPRAPWIEQEGPEYWDG |
| | | ETRKVKAHSQTHRVDLGTLRGYYNQ |
| | | SEAGSHTVQRMYGCDVGSDWRFLRG |
| | | YHQYAYDGKDYIALKEDLRSWTAAD |
| | | MAAQTTKHKWEAAHVAEQLRAYLEG |
| | | TCVEWLRRYLENGKETLQRTDAPKT |
| | | HMTHHAVSDHEATLRCWALSFYPAE |
| | | ITLTWQRDGEDQTQDTELVETRPAG |
| | | DGTFQKWAAVVVPSGQEQRYTCHVQ |
| | | HEGLPKPLTLRWEENLYFQGHHHHH |
| | | H |
| Single chain | 2 | MSRSVALAVLALLSLSGLEAGILGF |
| trimer | | VFTLGGGGSGGGGSGGGGSIQRTPK |
| | | IQVYSRHPAENGKSNFLNCYVSGFH |
| | | PSDIEVDLLKNGERIEKVEHSDLSF |
| | | SKDWSFYLLYYTEFTPTEKDEYACR |
| | | VNHVTLSQPKIVKWDRDMGGGGSGG |
| | | GGSGGGGSGGGGSGSHSMRYFFTSV |
| | | SRPGRGEPRFIAVGYVDDTQFVRFD |
| | | SDAASQRMEPRAPWIEQEGPEYWDG |
| | | ETRKVKAHSQTHRVDLGTLRGYYNQ |
| | | SEAGSHTVQRMYGCDVGSDWRFLRG |
| | | YHQYAYDGKDYIALKEDLRSWTAAD |
| | | MAAQTTKHKWEAAHVAEQLRAYLEG |
| | | TCVEWLRRYLENGKETLQRTDAPKT |
| | | HMTHHAVSDHEATLRCWALSFYPAE |
| | | ITLTWQRDGEDQTQDTELVETRPAG |
| | | DGTFQKWAAVVVPSGQEQRYTCHVQ |
| | | HEGLPKPLTLRWEPSSQPTIPIVGI |
| | | IAGLVLFGAVITGAVVAAVMWRRKS |
| | | SDRKGGSYSQAASSDSAQGSDVSLT |
| | | ACKV |
| Single chain | 3 | GILGFVFTLGGGGSGGGGSGGGGSI |
| trimer | | QRTPKIQVYSRHPAENGKSNFLNCY |
| | | VSGFHPSDIEVDLLKNGERIEKVEH |
| | | SDLSFSKDWSFYLLYYTEFTPTEKD |
| | | EYACRVNHVTLSQPKIVKWDRDMGG |
| | | GGSGGGGSGGGGSGGGGSGSHSMRY |
| | | FFTSVSRPGRGEPRFIAVGYVDDTQ |
| | | FVRFDSDAASQRMEPRAPWIEQEGP |
| | | EYWDGETRKVKAHSQTHRVDLGTLR |
| | | GYYNQSEAGSHTVQRMYGCDVGSDW |
| | | RFLRGYHQYAYDGKDYIALKEDLRS |
| | | WTAADMAAQTTKHKWEAAHVAEQLR |
| | | AYLEGTCVEWLRRYLENGKETLQRT |
| | | DAPKTHMTHHAVSDHEATLRCWALS |
| | | FYPAEITLTWQRDGEDQTQDTELVE |
| | | TRPAGDGTFQKWAAVVVPSGQEQRY |
| | | TCHVQHEGLPKPLTLRWEENLYFQG |
| | | HHHHHH |
| Single chain | 4 | GILGFVFTLGGGGSGGGGSGGGGSI |
| trimer | | QRTPKIQVYSRHPAENGKSNFLNCY |
| | | VSGFHPSDIEVDLLKNGERIEKVEH |
| | | SDLSFSKDWSFYLLYYTEFTPTEKD |
| | | EYACRVNHVTLSQPKIVKWDRDMGG |
| | | GGSGGGGSGGGGSGGGGSGSHSMRY |
| | | FFTSVSRPGRGEPRFIAVGYVDDTQ |
| | | FVRFDSDAASQRMEPRAPWIEQEGP |
| | | EYWDGETRKVKAHSQTHRVDLGTLR |
| | | GYYNQSEAGSHTVQRMYGCDVGSDW |
| | | RFLRGYHQYAYDGKDYIALKEDLRS |
| | | WTAADMAAQTTKHKWEAAHVAEQLR |
| | | AYLEGTCVEWLRRYLENGKETLQRT |
| | | DAPKTHMTHHAVSDHEATLRCWALS |
| | | FYPAEITLTWQRDGEDQTQDTELVE |
| | | TRPAGDGTFQKWAAVVVPSGQEQRY |
| | | TCHVQHEGLPKPLTLRWEENLYFQG |
| | | HHHHHH |
| Single chain | 5 | SYRPGTVALGGGGSGGGGSGGGGSI |
| trimer | | QRTPKIQVYSRHPAENGKSNFLNCY |
| | | VSGFHPSDIEVDLLKNGERIEKVEH |
| | | SDLSFSKDWSFYLLYYTEFTPTEKD |
| | | EYACRVNHVTLSQPKIVKWDRDMGG |
| | | GGSGGGGSGGGGSCSHSMRYFDTAV |
| | | SRPGRGEPRFISVGYVDDTQFVRFD |
| | | SDAASPRGEPRAPWVEQEGPEYWDR |
| | | ETQKYKRQAQADRVSLRNLRGYYNQ |
| | | SEDGSHTLQRMSGCDLGPDGRLLRG |
| | | YDQSAYDGKDYIALNEDLRSWTAAD |
| | | TAAQITQRKLEAARAAEQLRAYLEG |
| | | TCVEWLRRYLENGKETLQRAEPPKT |
| | | HVTHHPLSDHEATLRCWALGFYPAE |
| | | ITLTWQRDGEDQTQDTELVETRPAG |
| | | DGTFQKWAAVVVPSGQEQRYTCHMQ |
| | | HEGLQEPLTLSWEPSENLYFQGHHH |
| | | HHH |
| Single chain | 6 | SYRPGTVALGGGGSGGGGSGGGGSI |
| trimer | | QRTPKIQVYSRHPAENGKSNFLNCY |
| | | VSGFHPSDIEVDLLKNGERIEKVEH |
| | | SDLSFSKDWSFYLLYYTEFTPTEKD |
| | | EYACRVNHVTLSQPKIVKWDRDMGG |
| | | GGSGGGGSGGGGSGSHSMRYFDTAV |
| | | SRPGRGEPRFISVGYVDDTQFVRFD |
| | | SDAASPRGEPRAPWVEQEGPEYWDR |
| | | ETQKYKRQAQADRVSLRNLRGYYNQ |
| | | SEDGSHTLQRMSGCDLGPDGRLLRG |
| | | YDQSAYDGKDYIALNEDLRSWTAAD |
| | | TAAQITQRKLEAARAAEQLRAYLEG |
| | | TCVEWLRRYLENGKETLQRAEPPKT |
| | | HVTHHPLSDHEATLRCWALGFYPAE |
| | | ITLTWQRDGEDQTQDTELVETRPAG |
| | | DGTFQKWAAVVVPSGQEQRYTCHMQ |
| | | HEGLQEPLTLSWEPSENLYFQGHHH |
| | | HHH |
| Single chain | 7 | MSRSVALAVLALLSLSGLEAIQRTP |
| dimer | | KIQVYSRHPAENGKSNFLNCYVSGF |
| | | HPSDIEVDLLKNGERIEKVEHSDLS |
| | | FSKDWSFYLLYYTEFTPTEKDEYAC |
| | | RVNHVTLSQPKIVKWDRDMGGGGSG |
| | | GG |
| | | GSGGGGSGGGGSGSHSMRYFFTSVS |
| | | RPGRGEPRFIAVGYVDDTQFVRFDS |
| | | DAASQRMEPRAPWIEQEGPEYWDGE |
| | | TRKVKAHSQTHRVDLGTLRGYYNQS |
| | | EAGSHTVQRMYGCDVGSDWRFLRGY |
| | | HQYAYDGKDYIALKEDLRSWTAADM |
| | | AAQTTKHKWEAAHVAEQLRAYLEGT |
| | | CVEWLRRYLENGKETLQRTDAPKTH |
| | | MTHHAVSDHEATLRCWALSFYPAEI |
| | | TLTWQRDGEDQTQDTELVETRPAGD |
| | | GTFQKWAAVVVPSGQEQRYTCHVQH |
| | | EGLPKPLTLRWEPSSQPTIPIVGII |
| | | AGLVLFGAVITGAVVAAVMWRRKSS |
| | | DRKGGSYSQAASSDSAQGSDVSLTA |
| | | CKV |
| Single chain | 8 | SYRPGTVALGGGGSGGGGSIQRTPK |
| dimer | | IQVYSRHPAENGKSNFLNCYVSGFH |
| | | PSDIEVDLLKNGERIEKVEHSDLSF |
| | | SKDWSFYLLYYTEFTPTEKDEYACR |
| | | VNHVTLSQPKIVKWDRDM |
| Single chain | 9 | SYRPGTVALGCGGSGGGGSIQRTPK |
| dimer | | IQVYSRHPAENGKSNFLNCYVSGFH |
| | | PSDIEVDLLKNGERIEKVEHSDLSF |
| | | SKDWSFYLLYYTEFTPTEKDEYACR |
| | | VNHVTLSQPKIVKWDRDM |
| Single chain | 10 | SYRPGTVALGGGGSGGGGSGSIQRT |
| dimer | | PKIQVYSRHPAENGKSNFLNCYVSG |
| | | FHPSDIEVDLLKNGERIEKVEHSDL |
| | | SFSKDWSFYLLYYTEFTPTEKDEYA |
| | | CRVNHVTLSQPKIVKWDRDM |
| Single chain | 11 | SYRPGTVALGCGGSGGGGSGSIQRT |
| dimer | | PKIQVYSRHPAENGKSNFLNCYVSG |
| | | FHPSDIEVDLLKNGERIEKVEHSDL |
| | | SFSKDWSFYLLYYTEFTPTEKDEYA |
| | | CRVNHVTLSQPKIVKWDRDM |
| Single chain | 12 | SYRPGTVALGGGGSGGGGSGGGGSI |
| dimer | | QRTPKIQVYSRHPAENGKSNFLNCY |
| | | VSGFHPSDIEVDLLKNGERIEKVEH |
| | | SDLSFSKDWSFYLLYYTEFTPTEKD |
| | | EYACRVNHVTLSQPKIVKWDRDM |
| Single chain | 13 | SYRPGTVALGCGGSGGGGSGGGGSI |
| dimer | | QRTPKIQVYSRHPAENGKSNFLNCY |
| | | VSGFHPSDIEVDLLKNGERIEKVEH |
| | | SDLSFSKDWSFYLLYYTEFTPTEKD |
| | | EYACRVNHVTLSQPKIVKWDRDM |
| Synthetic | 14 | GILGFVFTLGGGGSGGGGSGGGGSI |
| HLA | | QRTPKIQVYSRHPAENGKSNFLNCY |
| | | VSGFHPSDIEVDLLKNGERIEKVEH |
| | | SDLSFSKDWSFYLLYYTEFTPTEKD |
| | | EYACRVNHVTLSQPKIVKWDRDMGG |
| | | GGSGGGGSGGGGSGSHSMRYFFTSV |
| | | SRPGRGEPRFIAVGYVDDTQFVRFD |
| | | SDAASQRMEPRAPWIEQEGPEYWDG |
| | | ETRKVKAHSQTHRVDLGTLRGAYNQ |
| | | SEAGSHTVQRMYGCDVGSDWRFLRG |
| | | YHQYAYDGKDYIALKEDLRSWTAAD |
| | | MAAQTTKHKWEAAHVAEQLRAYLEG |
| | | TCVEWLRRYLENGKETLQRTDAPKT |
| | | HMTHHAVSDHEATLRCWALSFYPAE |
| | | ITLTWQRDGEDQTQDTELVETRPAG |
| | | DGTFQKWAAVVVPSGQEQRYTCHVQ |
| | | HEGLPKPLTLRWEENLYFQGHHHHH |
| | | H |
| Synthetic | 15 | RYRPGTVALGGGGSGGGGSGGGGSI |
| HLA | | QRTPKIQVYSRHPAENGKSNFLNCY |
| | | VSGFHPSDIEVDLLKNGERIEKVEH |
| | | SDLSFSKDWSFYLLYYTEFTPTEKD |
| | | EYACRVNHVTLSQPKIVKWDRDMGG |
| | | GGSGGGGSGGGGSCSHSMRYFDTAV |
| | | SRPGRGEPRFISVGYVDDTQFVRFD |
| | | SDAASPRGEPRAPWVEQEGPEYWDR |
| | | ETQKYKRQAQADRVSLRNLRGAYNQ |
| | | SEDGSHTLQRMSGCDLGPDGRLLRG |
| | | YDQSAYDGKDYIALNEDLRSWTAAD |
| | | TAAQITQRKLEAARAAEQLRAYLEG |
| | | TCVEWLRRYLENGKETLQRAEPPKT |
| | | HVTHHPLSDHEATLRCWALGFYPAE |
| | | ITLTWQRDGEDQTQDTELVETRPAG |
| | | DGTFQKWAAVVVPSGQEQRYTCHMQ |
| | | HEGLQEPLTLSWEPSENLYFQGHHH |
| | | HHH |
| Synthetic | 16 | RYRPGTVALGGGGSGGGGSGGGGSI |
| HLA | | QRTPKIQVYSRHPAENGKSNFLNCY |
| | | VSGFHPSDIEVDLLKNGERIEKVEH |
| | | SDLSFSKDWSFYLLYYTEFTPTEKD |
| | | EYACRVNHVTLSQPKIVKWDRDMGG |
| | | GGSGGGGSGGGGSCSHSMRYFDTAV |
| | | SRPGRGEPRFISVGYVDDTQFVRFD |
| | | SDAASPRGEPRAPWVEQEGPEYWDR |
| | | ETQKYKRQAQADRVSLRNLRGAYNQ |
| | | SEDGSHTLQRMSGCDLGPDGRLLRG |
| | | YDQSAYDGKDYIALNEDLRSWTAAD |
| | | TAAQITQRKLEAARAAEQLRAYLEG |
| | | TCVEWLRRYLENGKETLQRAEPPKT |
| | | HVTHHPLSDHEATLRCWALGFYPAE |
| | | ITLTWQRDGEDQTQDTELVETRPAG |
| | | DGTFQKWAAVVVPSGQEQRYTCHMQ |
| | | HEGLQEPLTLSWEPSENLYFQGHHH |
| | | HHH |
| Synthetic | 17 | RYRPGTVALGGGGSGGGGSGGGGSI |
| HLA | | QRTPKIQVYSRHPAENGKSNFLNCY |
| | | VSGFHPSDIEVDLLKNGERIEKVEH |
| | | SDLSFSKDWSFYLLYYTEFTPTEKD |
| | | EYACRVNHVTLSQPKIVKWDRDMGG |
| | | GGSGGGGSGGGGSCSHSMRYFDTAV |
| | | SRPGRGEPRFISVGYVDDTQFVRFD |
| | | SDAASPRGEPRAPWVEQEGPEYWDR |
| | | ETQKYKRQAQADRVSLRNLRGAYNQ |
| | | SEDGSHTLQRMSGCDLGPDGRLLRG |
| | | YDQSAYDGKDYIALNEDLRSWTAAD |
| | | TAAQITQRKLEAARAAEQLRAYLEG |
| | | TCVEWLRRYLENGKETLQRAEPPKT |
| | | HVTHHPLSDHEATLRCWALGFYPAE |
| | | ITLTWQRDGEDQTQDTELVETRPAG |
| | | DGTFQKWAAVVVPSGQEQRYTCHMQ |
| | | HEGLQEPLTLSWEPSENLYFQGHHH |
| | | HHH |
| Synthetic | 18 | RYRPGTVALGGGGSGGGGSGSIQRT |
| HLA | | PKIQVYSRHPAENGKSNFLNCYVSG |
| | | FHPSDIEVDLLKNGERIEKVEHSDL |
| | | SFSKDWSFYLLYYTEFTPTEKDEYA |
| | | CRVNHVTLSQPKIVKWDRDMGGGGS |
| | | GGGGSGGGGSCSHSMRYFDTAVSRP |
| | | GRGEPRFISVGYVDDTQFVRFDSDA |
| | | ASPRGEPRAPWVEQEGPEYWDRETQ |
| | | KYKRQAQADRVSLRNLRGAYNQSED |
| | | GSHTLQRMSGCDLGPDGRLLRGYDQ |
| | | SAYDGKDYIALNEDLRSWTAADTAA |
| | | QITQRKLEAARAAEQLRAYLEGTCV |
| | | EWLRRYLENGKETLQRAEPPKTHVT |
| | | HHPLSDHEATLRCWALGFYPAEITL |
| | | TWQRDGEDQTQDTELVETRPAGDGT |
| | | FQKWAAVVVPSGQEQRYTCHMQHEG |
| | | LQEPLTLSWEPSENLYFQGHHHHHH |
| Synthetic | 19 | RYRPGTVALGGGGSGGGGSGSIQRT |
| HLA | | PKIQVYSRHPAENGKSNFLNCYVSG |
| | | FHPSDIEVDLLKNGERIEKVEHSDL |
| | | SFSKDWSFYLLYYTEFTPTEKDEYA |
| | | CRVNHVTLSQPKIVKWDRDMGGGGS |
| | | GGGGSGGGGSCSHSMRYFDTAVSRP |
| | | GRGEPRFISVGYVDDTQFVRFDSDA |
| | | ASPRGEPRAPWVEQEGPEYWDRETQ |
| | | KYKRQAQADRVSLRNLRGAYNQSED |
| | | GSHTLQRMSGCDLGPDGRLLRGYDQ |
| | | SAYDGKDYIALNEDLRSWTAADTAA |
| | | QITQRKLEAARAAEQLRAYLEGTCV |
| | | EWLRRYLENGKETLQRAEPPKTHVT |
| | | HHPLSDHEATLRCWALGFYPAEITL |
| | | TWQRDGEDQTQDTELVETRPAGDGT |
| | | FQKWAAVVVPSGQEQRYTCHMQHEG |
| | | LQEPLTLSWEPSENLYFQGHHHHHH |
| Synthetic | 20 | RYRPGTVALGGGGSGGGGSGSIQRT |
| HLA | | PKIQVYSRHPAENGKSNFLNCYVSG |
| | | FHPSDIEVDLLKNGERIEKVEHSDL |
| | | SFSKDWSFYLLYYTEFTPTEKDEYA |
| | | CRVNHVTLSQPKIVKWDRDMGGGGS |
| | | GGGGSGGGGSCSHSMRYFDTAVSRP |
| | | GRGEPRFISVGYVDDTQFVRFDSDA |
| | | ASPRGEPRAPWVEQEGPEYWDRETQ |
| | | KYKRQAQADRVSLRNLRGAYNQSED |
| | | GSHTLQRMSGCDLGPDGRLLRGYDQ |
| | | SAYDGKDYIALNEDLRSWTAADTAA |
| | | QITQRKLEAARAAEQLRAYLEGTCV |
| | | EWLRRYLENGKETLQRAEPPKTHVT |
| | | HHPLSDHEATLRCWALGFYPAEITL |
| | | TWQRDGEDQTQDTELVETRPAGDGT |
| | | FQKWAAVVVPSGQEQRYTCHMQHEG |
| | | LQEPLTLSWEPSENLYFQGHHHHHH |
| Synthetic | 21 | RYRPGTVALGGGGSGGGGSGSIQRT |
| HLA | | PKIQVYSRHPAENGKSNFLNCYVSG |
| | | FHPSDIEVDLLKNGERIEKVEHSDL |
| | | SFSKDWSFYLLYYTEFTPTEKDEYA |
| | | CRVNHVTLSQPKIVKWDRDMGGGGS |
| | | GGGGSGGGGSCSHSMRYFDTAVSRP |
| | | GRGEPRFISVGYVDDTQFVRFDSDA |
| | | ASPRGEPRAPWVEQEGPEYWDRETQ |
| | | KYKRQAQADRVSLRNLRGAYNQSED |
| | | GSHTLQRMSGCDLGPDGRLLRGYDQ |
| | | SAYDGKDYIALNEDLRSWTAADTAA |
| | | QITQRKLEAARAAEQLRAYLEGTCV |
| | | EWLRRYLENGKETLQRAEPPKTHVT |
| | | HHPLSDHEATLRCWALGFYPAEITL |
| | | TWQRDGEDQTQDTELVETRPAGDGT |
| | | FQKWAAVVVPSGQEQRYTCHMQHEG |
| | | LQEPLTLSWEPSENLYFQGHHHHHH |
| Synthetic | 22 | RYRPGTVALGGGGSGGGGSGSIQRT |
| HLA | | PKIQVYSRHPAENGKSNFLNCYVSG |
| | | FHPSDIEVDLLKNGERIEKVEHSDL |
| | | SFSKDWSFYLLYYTEFTPTEKDEYA |
| | | CRVNHVTLSQPKIVKWDRDMGGGGS |
| | | GGGGSGGGGSCSHSMRYFDTAVSRP |
| | | GRGEPRFISVGYVDDTQFVRFDSDA |
| | | ASPRGEPRAPWVEQEGPEYWDRETQ |
| | | KYKRQAQADRVSLRNLRGAYNQSED |
| | | GSHTLQRMSGCDLGPDGRLLRGYDQ |
| | | SAYDGKDYIALNEDLRSWTAADTAA |
| | | QITQRKLEAARAAEQLRAYLEGTCV |
| | | EWLRRYLENGKETLQRAEPPKTHVT |
| | | HHPLSDHEATLRCWALGFYPAEITL |
| | | TWQRDGEDQTQDTELVETRPAGDGT |
| | | FQKWAAVVVPSGQEQRYTCHMQHEG |
| | | LQEPLTLSWEPSENLYFQGHHHHHH |
| Synthetic | 23 | RYRPGTVALGGGGSGGGGSGSIQRT |
| HLA | | PKIQVYSRHPAENGKSNFLNCYVSG |
| | | FHPSDIEVDLLKNGERIEKVEHSDL |
| | | SFSKDWSFYLLYYTEFTPTEKDEYA |
| | | CRVNHVTLSQPKIVKWDRDMGGGGS |
| | | GGGGSGGGGSCSHSMRYFDTAVSRP |
| | | GRGEPRFISVGYVDDTQFVRFDSDA |
| | | ASPRGEPRAPWVEQEGPEYWDRETQ |
| | | KYKRQAQADRVSLRNLRGAYNQSED |
| | | GSHTLQRMSGCDLGPDGRLLRGYDQ |
| | | SAYDGKDYIALNEDLRSWTAADTAA |
| | | QITQRKLEAARAAEQLRAYLEGTCV |
| | | EWLRRYLENGKETLQRAEPPKTHVT |
| | | HHPLSDHEATLRCWALGFYPAEITL |
| | | TWQRDGEDQTQDTELVETRPAGDGT |
| | | FQKWAAVVVPSGQEQRYTCHMQHEG |
| | | LQEPLTLSWEPSENLYFQGHHHHHH |
| Synthetic | 24 | SYRPGTVALGGGGSGGGGSGGGGSI |
| HLA | | QRTPKIQVYSRHPAENGKSNFLNCY |
| | | VSGFHPSDIEVDLLKNGERIEKVEH |
| | | SDLSFSKDWSFYLLYYTEFTPTEKD |
| | | EYACRVNHVTLSQPKIVKWDRDMGG |
| | | GGSGGGGSGGGGSCSHSMRYFDTAV |
| | | SRPGRGEPRFISVGYVDDTQFVRFD |
| | | SDAASPRGEPRAPWVEQEGPEYWDR |
| | | ETQKYKRQAQADRVSLRNLRGAYNQ |
| | | SEDGSHTLQRMSGCDLGPDGRLLRG |
| | | YDQSAYDGKDYIALNEDLRSWTAAD |
| | | TAAQITQRKLEAARAAEQLRAYLEG |
| | | TCVEWLRRYLENGKETLQRAEPPKT |
| | | HVTHHPLSDHEATLRCWALGFYPAE |
| | | ITLTWQRDGEDQTQDTELVETRPAG |
| | | DGTFQKWAAVVVPSGQEQRYTCHMQ |
| | | HEGLQEPLTLSWEPSENLYFQGHHH |
| | | HHH |
| Synthetic | 25 | SYRPGTVALGGGGSGGGGSGGGGSI |
| HLA | | QRTPKIQVYSRHPAENGKSNFLNCY |
| | | VSGFHPSDIEVDLLKNGERIEKVEH |
| | | SDLSFSKDWSFYLLYYTEFTPTEKD |
| | | EYACRVNHVTLSQPKIVKWDRDMGG |
| | | GGSGGGGSGGGGSCSHSMRYFDTAV |
| | | SRPGRGEPRFISVGYVDDTQFVRFD |
| | | SDAASPRGEPRAPWVEQEGPEYWDR |
| | | ETQKYKRQAQADRVSLRNLRGAYNQ |
| | | SEDGSHTLQRMSGCDLGPDGRLLRG |
| | | YDQSAYDGKDYIALNEDLRSWTAAD |
| | | TAAQITQRKLEAARAAEQLRAYLEG |
| | | TCVEWLRRYLENGKETLQRAEPPKT |
| | | HVTHHPLSDHEATLRCWALGFYPAE |
| | | ITLTWQRDGEDQTAKTELVETRPAG |
| | | DGTFQKWAAVVVPSGQEQRYTCHMQ |
| | | HEGLQEPLTLSWEPSENLYFQGHHH |
| | | HHH |
| Synthetic | 26 | SYRPGTVALGCGGSGGGGSGGGGSI |
| HLA | | QRTPKIQVYSRHPAENGKSNFLNCY |
| | | VSGFHPSDIEVDLLKNGERIEKVEH |
| | | SDLSFSKDWSFYLLYYTEFTPTEKD |
| | | EYACRVNHVTLSQPKIVKWDRDMGG |
| | | GGSGGGGSGGGGSCSHSMRYFDTAV |
| | | SRPGRGEPRFISVGYVDDTQFVRFD |
| | | SDAASPRGEPRAPWVEQEGPEYWDR |
| | | ETQKYKRQAQADRVSLRNLRGCYNQ |
| | | SEDGSHTLQRMSGCDLGPDGRLLRG |
| | | YDQSAYDGKDYIALNEDLRSWTAAD |
| | | TAAQITQRKLEAARAAEQLRAYLEG |
| | | TCVEWLRRYLENGKETLQRAEPPKT |
| | | HVTHHPLSDHEATLRCWALGFYPAE |
| | | ITLTWQRDGEDQTAKTEL VETRPA |
| | | GDGTFQKWAAVVVPSGQEQRYTCHM |
| | | QHEGLQEPLTLSWEPSENLYFQGHH |
| | | HHHH |
| Synthetic | 27 | SYRPGTVALGGGGSGGGGSGSIQRT |
| HLA | | PKIQVYSRHPAENGKSNFLNCYVSG |
| | | FHPSDIEVDLLKNGERIEKVEHSDL |
| | | SFSKDWSFYLLYYTEFTPTEKDEYA |
| | | CRVNHVTLSQPKIVKWDRDMGGGGS |
| | | GGGGSGGGGSCSHSMRYFDTAVSRP |
| | | GRGEPRFISVGYVDDTQFVRFDSDA |
| | | ASPRGEPRAPWVEQEGPEYWDRETQ |
| | | KYKRQAQADRVSLRNLRGAYNQSED |
| | | GSHTLQRMSGCDLGPDGRLLRGYDQ |
| | | SAYDGKDYIALNEDLRSWTAADTAA |
| | | QITQRKLEAARAAEQLRAYLEGTCV |
| | | EWLRRYLENGKETLQRAEPPKTHVT |
| | | HHPLSDHEATLRCWALGFYPAEITL |
| | | TWQRDGEDQTQDTELVETRPAGDGT |
| | | FQKWAAVVVPSGQEQRYTCHMQHEG |
| | | LQEPLTLSWEPSENLYFQGHHHHHH |
| Synthetic | 28 | SYRPGTVALGGGGSGGGGSGSIQRT |
| HLA | | PKIQVYSRHPAENGKSNFLNCYVSG |
| | | FHPSDIEVDLLKNGERIEKVEHSDL |
| | | SFSKDWSFYLLYYTEFTPTEKDEYA |
| | | CRVNHVTLSQPKIVKWDRDMGGGGS |
| | | GGGGSGGGGSCSHSMRYFDTAVSRP |
| | | GRGEPRFISVGYVDDTQFVRFDSDA |
| | | ASPRGEPRAPWVEQEGPEYWDRETQ |
| | | KYKRQAQADRVSLRNLRGAYNQSED |
| | | GSHTLQRMSGCDLGPDGRLLRGYDQ |
| | | SAYDGKDYIALNEDLRSWTAADTAA |
| | | QITQRKLEAARAAEQLRAYLEGTCV |
| | | EWLRRYLENGKETLQRAEPPKTHVT |
| | | HHPLSDHEATLRCWALGFYPAEITL |
| | | TWQRDGEDQTAKTELVETRPAGDGT |
| | | FQKWAAVVVPSGQEQRYTCHMQHEG |
| | | LQEPLTLSWEPSENLYFQGHHHHHH |
| Synthetic | 29 | SYRPGTVALGCGGSGGGGSGSIQRT |
| HLA | | PKIQVYSRHPAENGKSNFLNCYVSG |
| | | FHPSDIEVDLLKNGERIEKVEHSDL |
| | | SFSKDWSFYLLYYTEFTPTEKDEYA |
| | | CRVNHVTLSQPKIVKWDRDMGGGGS |
| | | GGGGSGGGGSCSHSMRYFDTAVSRP |
| | | GRGEPRFISVGYVDDTQFVRFDSDA |
| | | ASPRGEPRAPWVEQEGPEYWDRETQ |
| | | KYKRQAQADRVSLRNLRGCYNQSED |
| | | GSHTLQRMSGCDLGPDGRLLRGYDQ |
| | | SAYDGKDYIALNEDLRSWTAADTAA |
| | | QITQRKLEAARAAEQLRAYLEGTCV |
| | | EWLRRYLENGKETLQRAEPPKTHVT |
| | | HHPLSDHEATLRCWALGFYPAEITL |
| | | TWQRDGEDQTAKTELVETRPAGDGT |
| | | FQKWAAVVVPSGQEQRYTCHMQHEG |
| | | LQEPLTLSWEPSENLYFQGHHHHHH |
| Synthetic | 30 | SYRPGTVALGGGGSGGGGSIQRTPK |
| HLA | | IQVYSRHPAENGKSNFLNCYVSGFH |
| | | PSDIEVDLLKNGERIEKVEHSDLSF |
| | | SKDWSFYLLYYTEFTPTEKDEYACR |
| | | VNHVTLSQPKIVKWDRDMGGGGSGG |
| | | GGSGGGGSCSHSMRYFDTAVSRPGR |
| | | GEPRFISVGYVDDTQFVRFDSDAAS |
| | | PRGEPRAPWVEQEGPEYWDRETQKY |
| | | KRQAQADRVSLRNLRGAYNQSEDGS |
| | | HTLQRMSGCDLGPDGRLLRGYDQSA |
| | | YDGKDYIALNEDLRSWTAADTAAQI |
| | | TQRKLEAARAAEQLRAYLEGTCVEW |
| | | LRRYLENGKETLQRAEPPKTHVTHH |
| | | PLSDHEATLRCWALGFYPAEITLTW |
| | | QRDGEDQTQDTELVETRPAGDGTFQ |
| | | KWAAVVVPSGQEQRYTCHMQHEGLQ |
| | | EPLTLSWEPSENLYFQGHHHHHH |
| Synthetic | 31 | SYRPGTVALGGGGSGGGGSIQRTPK |
| HLA | | IQVYSRHPAENGKSNFLNCYVSGFH |
| | | PSDIEVDLLKNGERIEKVEHSDLSF |
| | | SKDWSFYLLYYTEFTPTEKDEYACR |
| | | VNHVTLSQPKIVKWDRDMGGGGSGG |
| | | GGSGGGGSCSHSMRYFDTAVSRPGR |
| | | GEPRFISVGYVDDTQFVRFDSDAAS |
| | | PRGEPRAPWVEQEGPEYWDRETQKY |
| | | KRQAQADRVSLRNLRGAYNQSEDGS |
| | | HTLQRMSGCDLGPDGRLLRGYDQSA |
| | | YDGKDYIALNEDLRSWTAADTAAQI |
| | | TQRKLEAARAAEQLRAYLEGTCVEW |
| | | LRRYLENGKETLQRAEPPKTHVTHH |
| | | PLSDHEATLRCWALGFYPAEITLTW |
| | | QRDGEDQTAKTELVETRPAGDGTFQ |
| | | KWAAVVVPSGQEQRYTCHMQHEGLQ |
| | | EPLTLSWEPSENLYFQGHHHHHH |
| Synthetic | 32 | SYRPGTVALGCGGSGGGGSIQRTPK |
| HLA | | IQVYSRHPAENGKSNFLNCYVSGFH |
| | | PSDIEVDLLKNGERIEKVEHSDLSF |
| | | SKDWSFYLLYYTEFTPTEKDEYACR |
| | | VNHVTLSQPKIVKWDRDMGGGGSGG |
| | | GGSGGGGSCSHSMRYFDTAVSRPGR |
| | | GEPRFISVGYVDDTQFVRFDSDAAS |
| | | PRGEPRAPWVEQEGPEYWDRETQKY |
| | | KRQAQADRVSLRNLRGCYNQSEDGS |
| | | HTLQRMSGCDLGPDGRLLRGYDQSA |
| | | YDGKDYIALNEDLRSWTAADTAAQI |
| | | TQRKLEAARAAEQLRAYLEGTCVEW |
| | | LRRYLENGKETLQRAEPPKTHVTHH |
| | | PLSDHEATLRCWALGFYPAEITLTW |
| | | QRDGEDQTAKTELVETRPAGDGTFQ |
| | | KWAAVVVPSGQEQRYTCHMQHEGLQ |
| | | EPLTLSWEPSENLYFQGHHHHHH |
| Synthetic | 33 | SYRPGTVALGGGGSGGGGSGGGGSI |
| HLA | | QRTPKIQVYSRHPAENGKSNFLNCY |
| | | VSGFHPSDIEVDLLKNGERIEKVEH |
| | | SDLSFSKDWSFYLLYYTEFTPTEKD |
| | | EYACRVNHVTLSQPKIVKWDRDMGG |
| | | GGSGGGGSGGGGSGSHSMRYFDTAV |
| | | SRPGRGEPRFISVGYVDDTQFVRFD |
| | | SDAASPRGEPRAPWVEQEGPEYWDR |
| | | ETQKYKRQAQADRVSLRNLRGAYNQ |
| | | SEDGSHTLQRMSGCDLGPDGRLLRG |
| | | YDQSAYDGKDYIALNEDLRSWTAAD |
| | | TAAQITQRKLEAARAAEQLRAYLEG |
| | | TCVEWLRRYLENGKETLQRAEPPKT |
| | | HVTHHPLSDHEATLRCWALGFYPAE |
| | | ITLTWQRDGEDQTQDTELVETRPAG |
| | | DGTFQKWAAVVVPSGQEQRYTCHMQ |
| | | HEGLQEPLTLSWEPSENLYFQGHHH |
| | | HHH |
| Synthetic | 34 | SYRPGTVALGGGGSGGGGSGGGGSI |
| HLA | | QRTPKIQVYSRHPAENGKSNFLNCY |
| | | VSGFHPSDIEVDLLKNGERIEKVEH |
| | | SDLSFSKDWSFYLLYYTEFTPTEKD |
| | | EYACRVNHVTLSQPKIVKWDRDMGG |
| | | GGSGGGGSGGGGSGSHSMRYFDTAV |
| | | SRPGRGEPRFISVGYVDDTQFVRFD |
| | | SDAASPRGEPRAPWVEQEGPEYWDR |
| | | ETQKYKRQAQADRVSLRNLRGAYNQ |
| | | SEDGSHTLQRMSGCDLGPDGRLLRG |
| | | YDQSAYDGKDYIALNEDLRSWTAAD |
| | | TAAQITQRKLEAARAAEQLRAYLEG |
| | | TCVEWLRRYLENGKETLQRAEPPKT |
| | | HVTHHPLSDHEATLRCWALGFYPAE |
| | | ITLTWQRDGEDQTAKTEL VETRPA |
| | | GDGTFQKWAAVVVPSGQEQRYTCHM |
| | | QHEGLQEPLTLSWEPSENLYFQGHH |
| | | HHHH |
| Synthetic | 35 | SYRPGTVALGCGGSGGGGSGGGGSI |
| HLA | | QRTPKIQVYSRHPAENGKSNFLNCY |
| | | VSGFHPSDIEVDLLKNGERIEKVEH |
| | | SDLSFSKDWSFYLLYYTEFTPTEKD |
| | | EYACRVNHVTLSQPKIVKWDRDMGG |
| | | GGSGGGGSGGGGSGSHSMRYFDTAV |
| | | SRPGRGEPRFISVGYVDDTQFVRFD |
| | | SDAASPRGEPRAPWVEQEGPEYWDR |
| | | ETQKYKRQAQADRVSLRNLRGCYNQ |
| | | SEDGSHTLQRMSGCDLGPDGRLLRG |
| | | YDQSAYDGKDYIALNEDLRSWTAAD |
| | | TAAQITQRKLEAARAAEQLRAYLEG |
| | | TCVEWLRRYLENGKETLQRAEPPKT |
| | | HVTHHPLSDHEATLRCWALGFYPAE |
| | | ITLTWQRDGEDQTAKTELVETRPAG |
| | | DGTFQKWAAVVVPSGQEQRYTCHMQ |
| | | HEGLQEPLTLSWEPSENLYFQGHHH |
| | | HHH |
| Synthetic | 36 | SYRPGTVALGGGGSGGGGSGSIQRT |
| HLA | | PKIQVYSRHPAENGKSNFLNCYVSG |
| | | FHPSDIEVDLLKNGERIEKVEHSDL |
| | | SFSKDWSFYLLYYTEFTPTEKDEYA |
| | | CRVNHVTLSQPKIVKWDRDMGGGGS |
| | | GGGGSGGGGSGSHSMRYFDTAVSRP |
| | | GRGEPRFISVGYVDDTQFVRFDSDA |
| | | ASPRGEPRAPWVEQEGPEYWDRETQ |
| | | KYKRQAQADRVSLRNLRGAYNQSED |
| | | GSHTLQRMSGCDLGPDGRLLRGYDQ |
| | | SAYDGKDYIALNEDLRSWTAADTAA |
| | | QITQRKLEAARAAEQLRAYLEGTCV |
| | | EWLRRYLENGKETLQRAEPPKTHVT |
| | | HHPLSDHEATLRCWALGFYPAEITL |
| | | TWQRDGEDQTQDTELVETRPAGDGT |
| | | FQKWAAVVVPSGQEQRYTCHMQHEG |
| | | LQEPLTLSWEPSENLYFQGHHHHHH |
| Synthetic | 37 | SYRPGTVALGGGGSGGGGSGSIQRT |
| HLA | | PKIQVYSRHPAENGKSNFLNCYVSG |
| | | FHPSDIEVDLLKNGERIEKVEHSDL |
| | | SFSKDWSFYLLYYTEFTPTEKDEYA |
| | | CRVNHVTLSQPKIVKWDRDMGGGGS |
| | | GGGGSGGGGSGSHSMRYFDTAVSRP |
| | | GRGEPRFISVGYVDDTQFVRFDSDA |
| | | ASPRGEPRAPWVEQEGPEYWDRETQ |
| | | KYKRQAQADRVSLRNLRGAYNQSED |
| | | GSHTLQRMSGCDLGPDGRLLRGYDQ |
| | | SAYDGKDYIALNEDLRSWTAADTAA |
| | | QITQRKLEAARAAEQLRAYLEGTCV |
| | | EWLRRYLENGKETLQRAEPPKTHVT |
| | | HHPLSDHEATLRCWALGFYPAEITL |
| | | TWQRDGEDQTAKTELVETRPAGDGT |
| | | FQKWAAVVVPSGQEQRYTCHMQHEG |
| | | LQEPLTLSWEPSENLYFQGHHHHHH |
| Synthetic | 38 | SYRPGTVALGGGGSGGGGSGSIQRT |
| HLA | | PKIQVYSRHPAENGKSNFLNCYVSG |
| | | FHPSDIEVDLLKNGERIEKVEHSDL |
| | | SFSKDWSFYLLYYTEFTPTEKDEYA |
| | | CRVNHVTLSQPKIVKWDRDMGGGGS |
| | | GGGGSGGGGSGSHSMRYFDTAVSRP |
| | | GRGEPRFISVGYVDDTQFVRFDSDA |
| | | ASPRGEPRAPWVEQEGPEYWDRETQ |
| | | KYKRQAQADRVSLRNLRGAYNQSED |
| | | GSHTLQRMSGCDLGPDGRLLRGYDQ |
| | | SAYDGKDYIALNEDLRSWTAADTAA |
| | | QITQRKLEAARAAEQLRAYLEGTCV |
| | | EWLRRYLENGKETLQRAEPPKTHVT |
| | | HHPLSDHEATLRCWALGFYPAEITL |
| | | TWQRDGEDQTAKTELVETRPAGDGT |
| | | FQKWAAVVVPSGQEQRYTCHMQHEG |
| | | LQEPLTLSWEPSENLYFQGHHHHHH |
| Synthetic | 39 | SYRPGTVALGGGGSGGGGSIQRTPK |
| HLA | | IQVYSRHPAENGKSNFLNCYVSGFH |
| | | PSDIEVDLLKNGERIEKVEHSDLSF |
| | | SKDWSFYLLYYTEFTPTEKDEYACR |
| | | VNHVTLSQPKIVKWDRDMGGGGSGG |
| | | GGSGGGGSGSHSMRYFDTAVSRPGR |
| | | GEPRFISVGYVDDTQFVRFDSDAAS |
| | | PRGEPRAPWVEQEGPEYWDRETQKY |
| | | KRQAQADRVSLRNLRGAYNQSEDGS |
| | | HTLQRMSGCDLGPDGRLLRGYDQSA |
| | | YDGKDYIALNEDLRSWTAADTAAQI |
| | | TQRKLEAARAAEQLRAYLEGTCVEW |
| | | LRRYLENGKETLQRAEPPKTHVTHH |
| | | PLSDHEATLRCWALGFYPAEITLTW |
| | | QRDGEDQTQDTELVETRPAGDGTFQ |
| | | KWAAVVVPSGQEQRYTCHMQHEGLQ |
| | | EPLTLSWEPSENLYFQGHHHHHH |
| Synthetic | 40 | SYRPGTVALGGGGSGGGGSIQRTPK |
| HLA | | IQVYSRHPAENGKSNFLNCYVSGFH |
| | | PSDIEVDLLKNGERIEKVEHSDLSF |
| | | SKDWSFYLLYYTEFTPTEKDEYACR |
| | | VNHVTLSQPKIVKWDRDMGGGGSGG |
| | | GGSGGGGSGSHSMRYFDTAVSRPGR |
| | | GEPRFISVGYVDDTQFVRFDSDAAS |
| | | PRGEPRAPWVEQEGPEYWDRETQKY |
| | | KRQAQADRVSLRNLRGAYNQSEDGS |
| | | HTLQRMSGCDLGPDGRLLRGYDQSA |
| | | YDGKDYIALNEDLRSWTAADTAAQI |
| | | TQRKLEAARAAEQLRAYLEGTCVEW |
| | | LRRYLENGKETLQRAEPPKTHVTHH |
| | | PLSDHEATLRCWALGFYPAEITLTW |
| | | QRDGEDQTQDTELVETRPAGDGTFQ |
| | | KWAAVVVPSGQEQRYTCHMQHEGLQ |
| | | EPLTLSWEPSENLYFQGHHHHHH |
| Synthetic | 41 | SYRPGTVALGCGGSGGGGSIQRTPK |
| HLA | | IQVYSRHPAENGKSNFLNCYVSGFH |
| | | PSDIEVDLLKNGERIEKVEHSDLSF |
| | | SKDWSFYLLYYTEFTPTEKDEYACR |
| | | VNHVTLSQPKIVKWDRDMGGGGSGG |
| | | GGSGGGGSGSHSMRYFDTAVSRPGR |
| | | GEPRFISVGYVDDTQFVRFDSDAAS |
| | | PRGEPRAPWVEQEGPEYWDRETQKY |
| | | KRQAQADRVSLRNLRGCYNQSEDGS |
| | | HTLQRMSGCDLGPDGRLLRGYDQSA |
| | | YDGKDYIALNEDLRSWTAADTAAQI |
| | | TQRKLEAARAAEQLRAYLEGTCVEW |
| | | LRRYLENGKETLQRAEPPKTHVTHH |
| | | PLSDHEATLRCWALGFYPAEITLTW |
| | | QRDGEDQTAKTELVETRPAGDGTFQ |
| | | KWAAVVVPSGQEQRYTCHMQHEGLQ |
| | | EPLTLSWEPSENLYFQGHHHHHH |
| Synthetic | 42 | GSHSMRYFDTAVSRPGRGEPRFISV |
| HLA | and | GYVDDTQFVRFDSDAASP |
| | 55 | RGEPRAPWVEQEGPEYWDRETQKYK |
| | | RQAQADRVSLRNLRGAYNQSEDGSH |
| | | TLQRMSGCDLGPDGRLLRGYDQSAY |
| | | DGKDYIALNEDLRSWTAADTAAQIT |
| | | QRKLEAARAAEQLRAYLEGTCVEWL |
| | | RRYLENGKETLQRAEPPKTHVTHHP |
| | | LSDHEATLRCWALGFYPAEITLTWQ |
| | | RDGEDQTQDTELVETRPAGDGTFQK |
| | | WAAVVVPSGQEQRYTCHMQHEGLQE |
| | | PLTLSWEPSENLYFQGHHHHHH- |
| | | stop-GLNDIFEAQKIEWHE |
| Synthetic | 43 | GSHSMRYFDTAVSRPGRGEPRFISV |
| HLA | and | GYVDDTQFVRFDSDAASP |
| | 56 | RGEPRAPWVEQEGPEYWDRETQKYK |
| | | RQAQADRVSLRNLRGAYNQSEDGSH |
| | | TLQRMSGCDLGPDGRLLRGYDQSAY |
| | | DGKDYIALNEDLRSWTAADTAAQIT |
| | | QRKLEAARAAEQLRAYLEGTCVEWL |
| | | RRYLENGKETLQRAEPPKTHVTHHP |
| | | LSDHEATLRCWALGFYPAEITLTWQ |
| | | RDGEDQTAKTELVETRPAGDGTFQK |
| | | WAAVVVPSGQEQRYTCHMQHEGLQE |
| | | PLTLSWEPSENLYFQGHHHHHH- |
| | | stop-GLNDIFEAQKIEWHE |
| Synthetic | 44 | GSHSMRYFDTAVSRPGRGEPRFISV |
| HLA | and | GYVDDTQFVRFDSDAASPRGEPRAP |
| | 57 | WVEQEGPEYWDRETQKYKRQAQADR |
| | | VSLRNLRGCYNQSEDGSHTLQRMSG |
| | | CDLGPDGRLLRGYDQSAYDGKDYIA |
| | | LNEDLRSWTAADTAAQITQRKLEAA |
| | | RAAEQLRAYLEGTCVEWLRRYLENG |
| | | KETLQRAEPPKTHVTHHPLSDHEAT |
| | | LRCWALGFYPAEITLTWQRDGEDQT |
| | | AKTELVETRPAGDGTFQKWAAVVVP |
| | | SGQEQRYTCHMQHEGLQEPLTLSWE |
| | | PSENLYFQGHHHHHH-stop-GLND |
| | | IFEAQKIEWHE |

### Example 9: Expression of Synthetic HLA Constructs in Bacteria

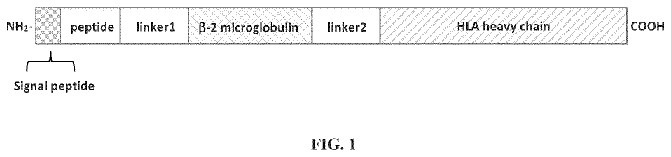

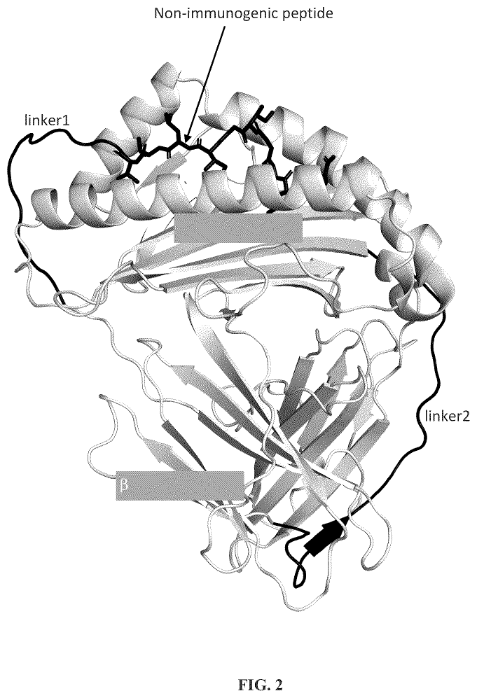

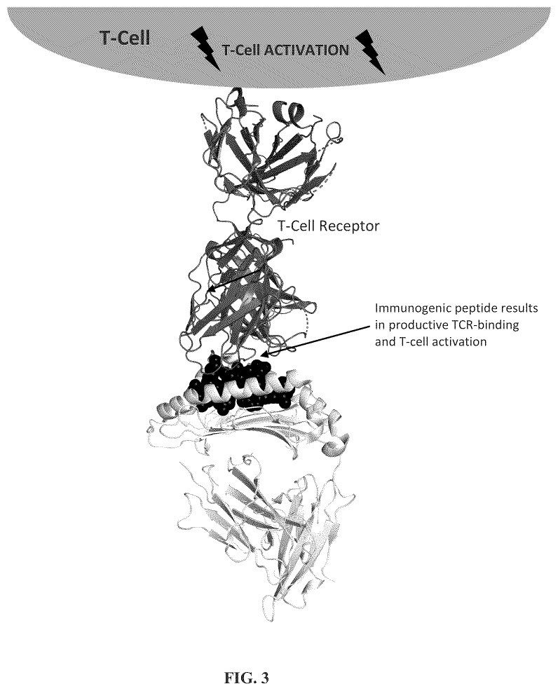

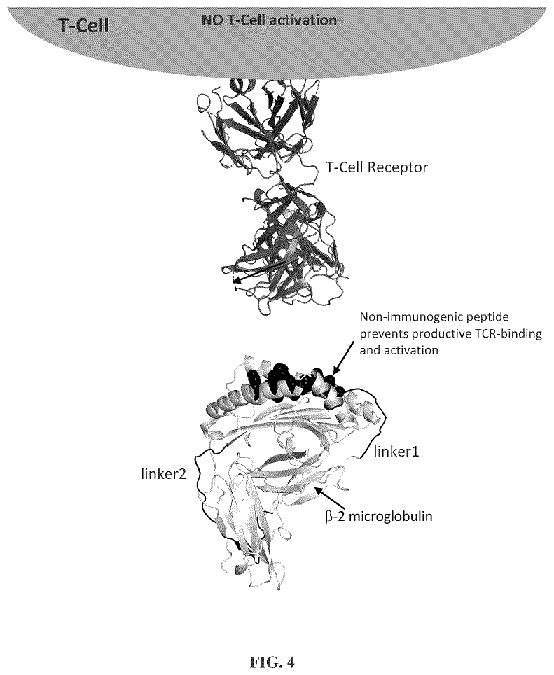

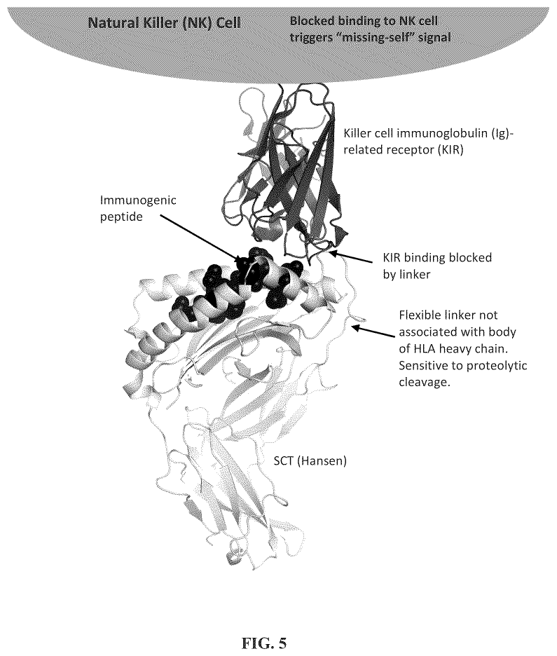

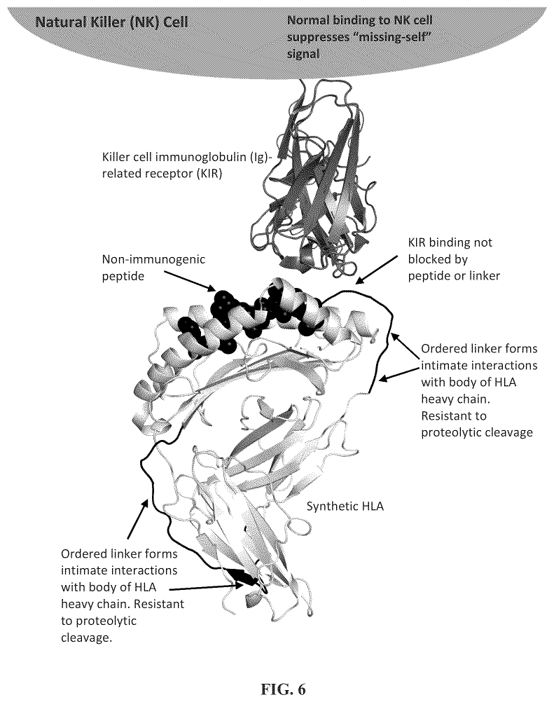

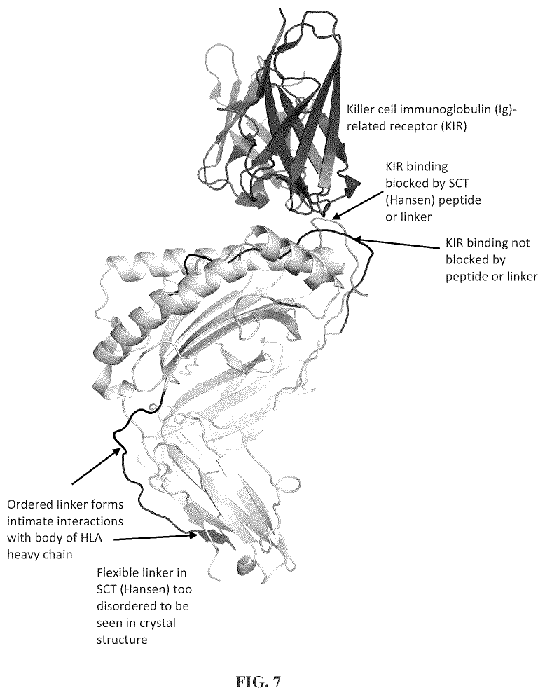

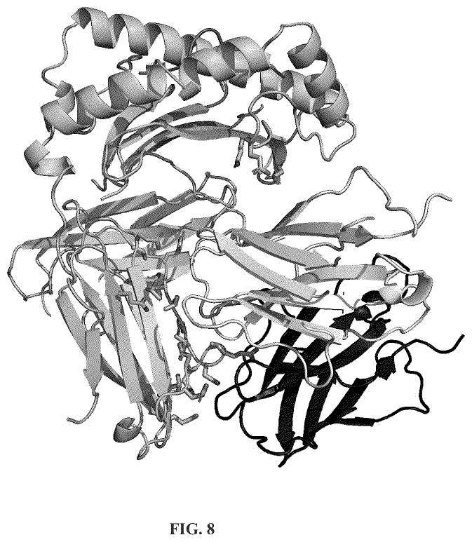

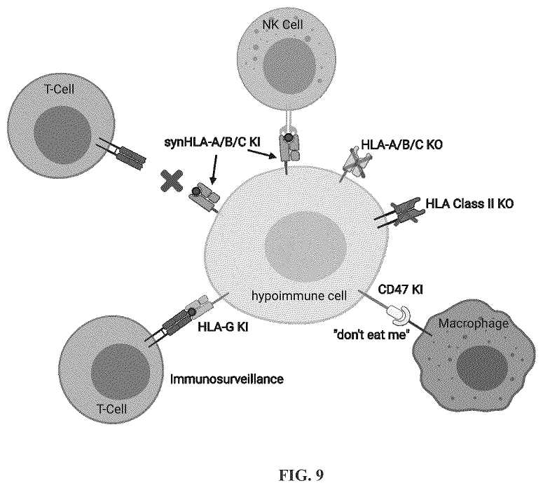

Synthetic HLA proteins as described herein (SYNC4-1, SEQ ID NO: 18; SYNA1-1, SEQ ID NO: 14; SYNC5-1, SEQ ID NO: 19; SYNC6-1, SEQ ID NO:20; SCTC1-1, SEQ ID NO:4) were expressed, isolated, refolded, purified, and analyzed as described in Example 8. Briefly, DNA encoding synthetic HLA proteins was inserted into an expression vector, then introduced into bacteria under antibiotic selection. The protein construct format was as described in FIG. 1, but with no N-terminal signal peptide and an additional C-terminal hexa-His tag for purification. Cells harboring the appropriate sequences were grown to mid-log phase, and expression was induced with isopropyl β-D-1-thiogalactopyranoside (IPTG). Cells were harvested by centrifugation, lysed using a high-pressure homogenizer and centrifuged to isolate the soluble and insoluble fractions. Insoluble inclusion bodies containing synthetic HLA proteins were washed and resuspended in a denaturing buffer. Synthetic HLA proteins were refolded from the inclusion body preparation by dilution in a refold buffer, followed by incubation at 4° C. for up to 72 hours. The resulting protein was purified by a combination of IMAC, size exclusion and/or ion exchange.

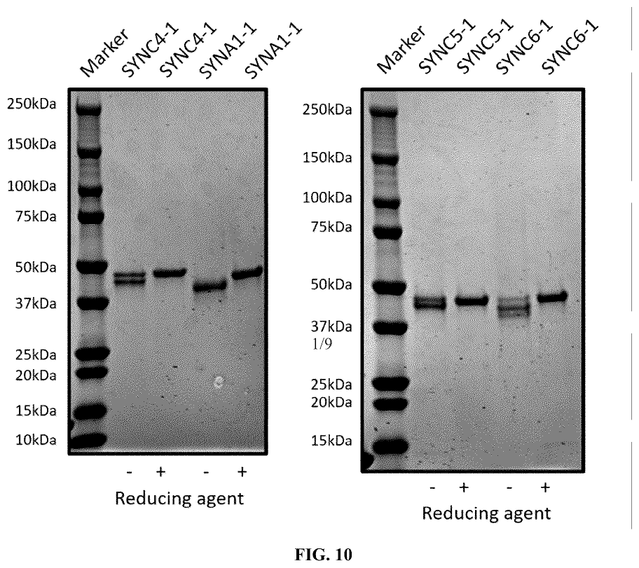

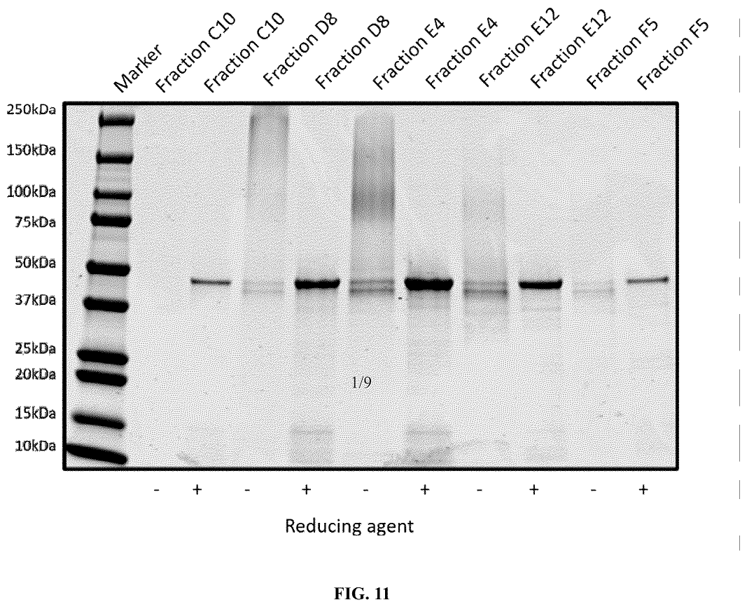

Results of the SDS-PAGE analysis of each construct are shown in FIGS. 10 and 11. Separation by SDS-PAGE followed by Coomassie blue staining shows that SYNA1-1 migrates as a single band under non-reducing conditions suggesting homogeneity of disulfite bond formation (FIG. 10). SYNA1-1 contains the HLA-A02 scaffold. The synthetic HLA proteins SCTC1-1, SYNC4-1, SYNC5-1 and SYNC6-1, which contain the HLA-007 scaffold, migrate as multiple species under non-reducing conditions (FIGS. 10 and 11). This suggests that multiple species of disulfide-bonded proteins are present. This may be a result of an extra unpaired cysteine present in the HLA-007 domain.

### Example 10: Modulating Possibility of Disulfide Bond Formation

The cysteine present in the HLA-007 domain as described in Example 9 is mutated to another amino acid, potentially reducing or ablating the possibility or mis-paired disulfides forming and thus improving protein expression. Other mutations may be introduced to improve expression and/or refolding as measured by techniques described herein.

### Example 11: Thermal Stability Assay

A thermal stability assay was carried out to monitor the unfolding of proteins by applying a thermal gradient from 10-100° C. at 1.0° C. per minute on a Bio-Rad CFX96™ Real-Time System RT PCR. Protein unfolding was measured by monitoring the increase in signal from a fluorescent dye, SYPRO™ orange, which binds to hydrophobic regions of proteins as they unfold. Proteins were assayed at 5 μM and were measured individually and in combination as described below. Determination of the melting point, Tₘ (the temperature of the midpoint of the melt transition), of the proteins was done using the Bio-Rad CFX manager software. The first derivative (negative mode) of the protein melt curve identifies maxima that correspond to the Tₘ, a one-step protein unfolding event would have a single maximum, while a two-step unfolding event would have two maxima, and so on.

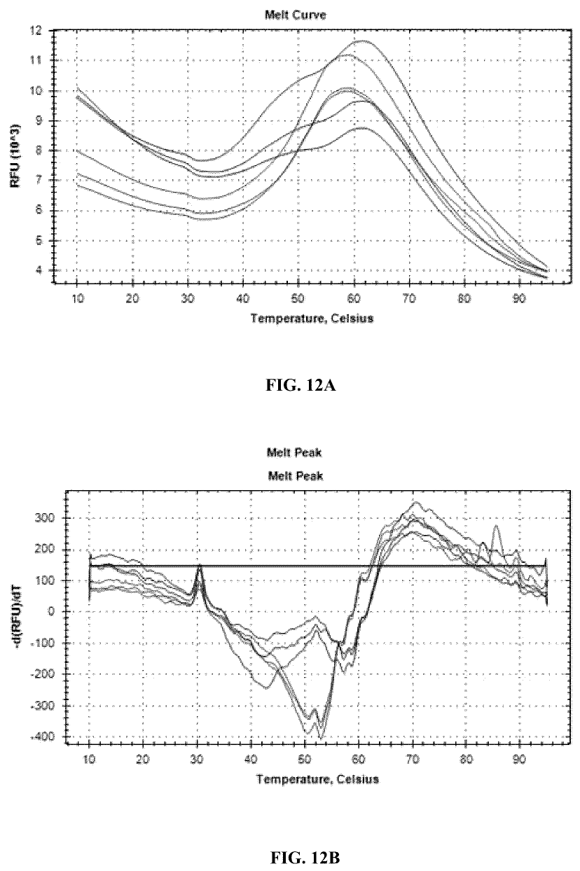

The melt curve of SYNC4-1 showed a pronounced two-step unfolding event (FIG. 12A), corresponding to a Tₘ of 42.6° C. & 57.2° C., while the combination of SYNC4-1 and KIR2DL2 results in a major shift to the right of the first maximum corresponding to a Tₘ of 54.8° C. and 57.4° C. The pronounced double maxima of SYNC4-1 alone became a condensed double peak (FIG. 12B). The shift in Tₘ to the right suggests the KIR2DL2 stabilized SYNC4-1 through protein-protein interaction.

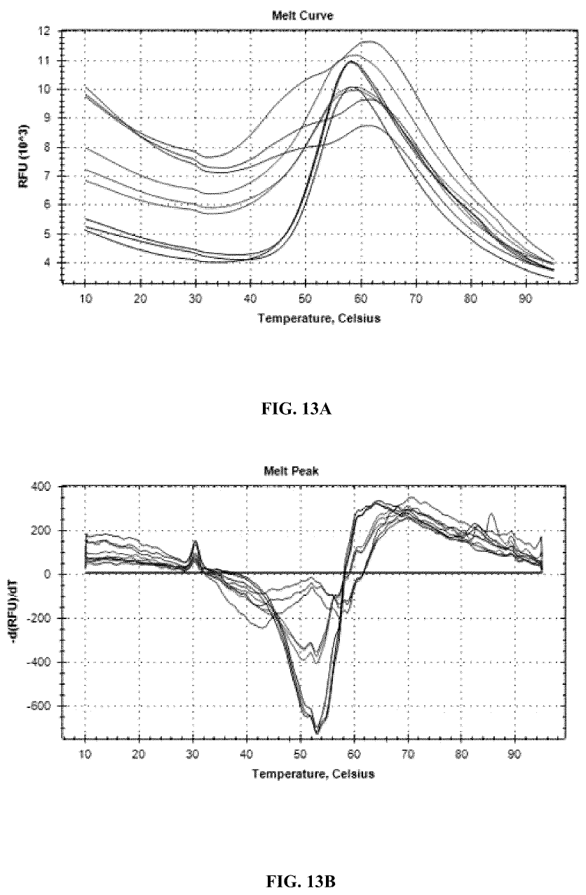

In comparison to the KIR2DL2 melt curve and first derivative (FIG. 13), the two maxima have further condensed to a single maximum with a slight shoulder on the left hand side, which correspond to a Tₘ 58.8° C. and 57.6° C. respectively. Once again, this strongly suggests there is a protein-protein interaction between SYNC4-1 and KIR2DL2.

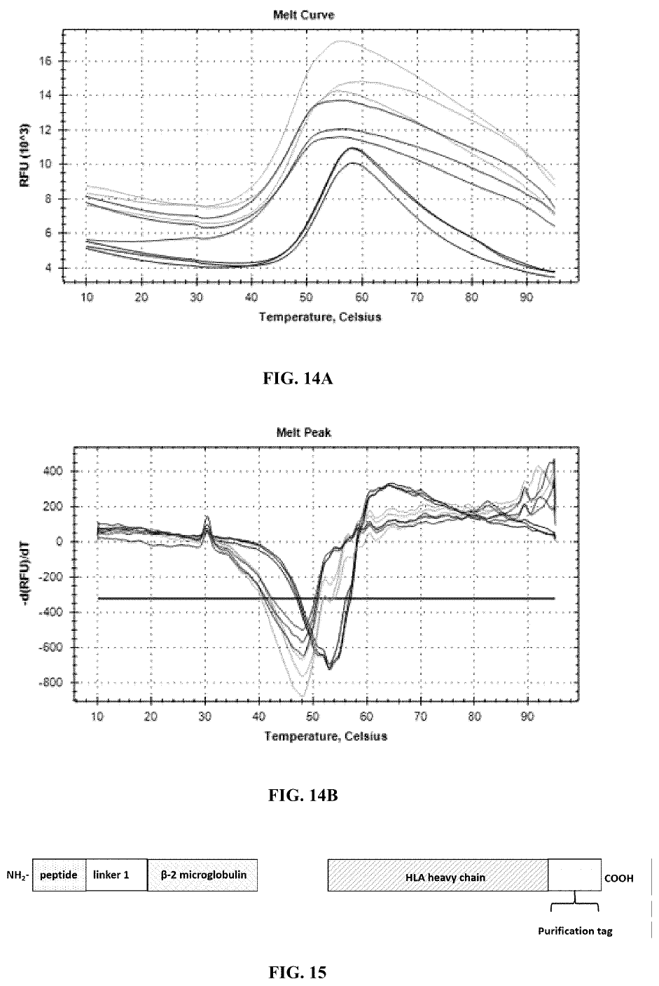

In contrast to SYNC1-1, SYNA1-1 which has the influenza peptide GILGFVFTL and a relatively long linker sequence that may interfere with MR-binding, did not lead to a shift to the right when combined with KIR2DL2 (FIG. 14), indicating KIR2DL2 did not increase the thermal stability of SYNA1-1 (SYNA1-1 Tₘ=48° C., SYNA1-1+KIR2DL2 Tₘ=48.2° C.). Instead, the thermal stability of KIR2DL2 was reduced when combined with SYNA1-1 (FIG. 14), Tₘ of 58° C. and 48° C. respectively. These data suggest that there was no increase in thermal stability when combining SYNA1-1 with KIR2DL2, suggesting little to no interactions between the two proteins.

### Example 12: Interaction of Soluble Synthetic HLA Proteins with Immune Receptors

To investigate the ability of soluble synthetic HLA proteins to evade the immune system, their interaction with immune receptors is examined. Recombinant immune receptors such as the killer cell immunoglobulin-like receptor (MR) on natural killer cells, and the T-cell receptor and CD8 co-receptor on T-cells are tested. Standard protein-protein interaction techniques such as surface plasmon resonance, enzyme linked immunosorbent assay (ELISA), thermal melt assays, and circular dichroism are used to investigate this interaction. When compared to controls, soluble synthetic HLA proteins harboring specific mutations exhibit impaired binding to recombinant immune receptors.

To examine the interaction of soluble synthetic HLA proteins with immune receptors at the cell surface, competitive cellular assays are used. In these assays, soluble synthetic HLA proteins are incubated with mammalian cells expressing wild type Class I HLA molecules. The ability of the wild type cells to survive in the presence of immune cells (e.g. T-cells and/or NK cells) is then measured. When compared to controls, soluble HLA proteins harboring specific mutations are less reactive with the receptors on immune cells, resulting in increased killing of the wild type mammalian cells.

### Example 13: Production of Soluble Synthetic HLA Proteins from Separate Transcriptional Units

DNA encoding synthetic HLA proteins is inserted into a vector, then introduced into bacteria under antibiotic selection. The construct format is as described in FIG. 15 Briefly, the peptide and β-2 microglobulin domains are expressed as a single protein joined by a linker, and the HLA heavy chain domain is produced as a separate protein. This may be within the same cell or separate cells. Cells harboring the appropriate constructs are grown to mid-log phase, and expression is induced with isopropyl β-D-1-thiogalactopyranoside (IPTG). Cells are harvested by centrifugation, lysed using a cell crusher and centrifuged to isolate the soluble and insoluble fractions. Insoluble inclusion bodies containing the appropriate domains of the synthetic HLA proteins are washed and resuspended in a denaturing buffer. The peptide and β-2 microglobulin protein is combined with the HLA heavy chain domain during a refolding step. The resulting refolded protein is purified by a combination of IMAC, size exclusion and/or ion exchange. Alternatively, the refolded protein is purified directly from the soluble fraction using similar purification techniques.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

1-121. (canceled)

122. A complex comprising: a segment comprising a peptide, one or more linkers, and a segment comprising a human HLA class 1 heavy chain sequence, wherein said human HLA class 1 heavy chain sequence comprises HLA-C, wherein said human HLA class 1 heavy chain sequence comprises one or more mutations occurring at one or more of amino acid residues 115, 122, 128, 194, 197, 198, 212, 214, 222, 223, 224, 225, 226, 227, 228, 229, 230, 231, 232, 233, 243, 245, 248, 262, or any combination thereof, compared to an endogenous HLA of the same type.

123. The complex of claim 122, wherein said peptide is coupled to one or more HLA binding groove domain residues of said human HLA class 1 heavy chain sequence.

124. The complex of claim 122, wherein said peptide modulates a conformation of said human HLA class 1 heavy chain sequence.

125. The complex of claim 122, wherein a linker of said one or more linkers is disposed between said peptide and said human HLA class 1 heavy chain sequence, wherein said linker is configured to resist proteolytic cleavage, and wherein said linker comprises a conformation configured to not block one or more killer cell immunoglobulin like receptor binding sites on said human HLA class 1 heavy chain sequence.

126. The complex of claim 122, wherein said human HLA class 1 heavy chain sequence is inhibited from eliciting a T-cell response when said complex is interrogated by one or more T cells.

127. The complex of claim 122, wherein said peptide is coupled to said complex by a disulfide bond.

128. The complex of claim 122, wherein said complex further comprises one or more immune checkpoint agonists.

129. The complex of claim 128, wherein said one or more immune checkpoint agonists comprise CD47, PD-L1, A2AR, B7-H3, B7-H4, BTLA, CTLA-4, IDO, KIR, LAG3, NOX2, PD-1, TIM-3, VISTA, SIGLEC7, or a combination thereof.

130. The complex of claim 122, wherein a linker of said one or more linkers is disposed between said peptide and a human β2-microglobulin sequence, between a human β2-microglobulin sequence and said human HLA class 1 heavy chain sequence, or both.

131. The complex of claim 122, wherein a linker of said one or more linkers comprises a sequence at least about 90% identical to any one of SEQ ID NOs: 48-54.

132. The complex of claim 122, wherein said complex further comprises a synthetic HLA-E or fragment thereof.

133. The complex of claim 132, wherein said synthetic HLA-E or fragment thereof comprises one or more mutations as compared to an endogenous HLA-E or equivalent fragment thereof.

134. The complex of claim 122, wherein said peptide comprises an amino acid sequence comprising nine amino acid residues, and wherein said peptide comprises, in N-terminus to C-terminus order:
a. an amino acid residue selected from E, P, L, Q, A, R, H, S, T, V, M, D, and K at a second position of said amino acid sequence; and
b. an amino acid residue selected from V, I, F, W, Y, L, R, and K at a ninth position of said amino acid sequence.

135. An isolated cell comprising a nucleic acid molecule encoding the complex of claim 122.

136. The isolated cell of claim 135, wherein said isolated cell is a stem cell.

137. The isolated cell of claim 136, wherein said stem cell is an embryonic stem cell, a hematopoietic stem cell, a mesenchymal stem cell, or an induced pluripotent stem cell.

138. A method of generating an immune incompetent cell, comprising administering a nucleic acid molecule encoding the complex of claim 122.

139. The method of claim 138, comprising administering the nucleic acid molecule to a stem cell.

140. The method of claim 139, wherein said stem cell is an embryonic stem cell, a hematopoietic stem cell, a mesenchymal stem cell, or an induced pluripotent stem cell.
